(19) United States
(12) Patent Application Publication
(10) Pub. No.: US 2017/0173086 A1
(43) Pub. Date: Jun. 22, 2017

(54) Methods and Genetic Systems for Cell Engineering

(71) Applicant: GINKGO BIOWORKS, INC., Boston, MA (US)

(72) Inventors: Patrick Boyle, Worcester, MA (US); Reshma Shetty, Boston, MA (US)

(21) Appl. No.: 15/127,498

(22) PCT Filed: Mar. 25, 2015
(86) PCT No.: PCT/US15/22508

§ 371 Date: Sep. 20, 2016

Related U.S. Application Data
(60) Provisional application No. 61/970,024, filed on Mar. 25, 2014.

(51) Int. Cl. A61K 35/741; A61K 35/747; C12N 9/22

(52) U.S. Cl. CPC A61K 35/741; C12N 9/22; A61K 35/747; A61K 2035/115

(57) ABSTRACT

The present disclosure provides engineered genetic systems and methods to confer the ability to target and degrade undesirable nuclic acids in an organism so as to combat gastrointestinal, skin or urinary tract disease and infection, prevent the spread of antibiotic resistance, and/or decontaminate environmental pathogens. The engineered genetic system can also be used for the therapeutic treatment of humans and animals. The undesirable nucleic acids can be DNA and/or RNA.

### CROSS REFERENCE TO RELATED APPLICATIONS

This application claims priority to and the benefit of U.S. Provisional Application No. 61/970,024 filed Mar. 25, 2014, the entire disclosure of which is hereby incorporated by reference.

### STATEMENT REGARDING GOVERNMENT LICENSE RIGHTS

This invention was made with government support under contract number W31P4Q-13-C-0063 awarded by U.S. Defense Advanced Research Projects Agency (DARPA) SBIR program. The government has certain rights in the invention.

### TECHNICAL FIELD

The disclosure relates to systems, mechanisms and methods to develop an engineered genetic system that can be introduced into a cell such as a probiotic for gene editing. The enginerred probiotic can be used in, for example, the treatment of gastrointestinal, skin or urinary tract diseases and infections, combatting the spread of antibiotic resistance, and decontamination of environmental pathogens.

### BACKGROUND

Next generation sequencing technologies are allowing researchers to rapidly and accurately interrogate the genomic content of microbiomes and catalog both commensal and pathogenic microbes. Advances in our understanding of the mammalian microbiome are likely to lead to the use of next generation sequencing as a diagnostic tool to identify the existence and precise genotype of pathogens and virulence genes and distinguish between the microbiome composition and structure of healthy and diseased individuals.

Likewise, our ability to engineer microbes using synthetic biology and metabolic engineering tools and technologies has advanced to the point where we can begin to consider applying engineered microbes to restore healthy microbiome states. Engineered organisms are already used widely in human and veterinary health for therapeutic production, biomedical research, and more recently as products themselves. It is now possible to merge the fields of microbiome discovery and synthetic biology to develop new strategies to modulate human and animal health and disease.

One area of growing opportunity in this field is translating microbiome-based discovery into therapeutic impact by employing probiotics to modulate diseases and infections with a microbial component. Although not totally understood, many skin disorders are believed to have a microbial component. Lesions resulting from atopic dermatitis often become infected with pathogens like Staphylococcus aureus. Seborrhoeic dermatitis is believed to have a fungal component since treatment with fungicides is effective. Burn wounds are often infected with Streptococcus pyogenes, Enterococcus spp., or Pseudomonas aeruginosa.

Currently, many gastrointestinal disease states have been associated with changes in the composition of faecal and intestinal mucosal communities, including inflammatory bowel diseases (IBD and IBS), obesity and the metabolic syndrome. Probiotics, or beneficial microbes, are used to improve symbiosis between enteric microbiota and the host or to restore states of dysbiosis. Probiotics may modulate immune responses, provide key nutrients, or suppress the proliferation and virulence of infectious agents. In particular, the enteric microbiota are known to impact gastrointestinal health and the disruption of this homeostasis is associated with many disease states such as diarrhea. Diarrhea is defined by the WHO as the condition of having three or more loose or liquid bowel movements per day. The disease can be acute—usually due to an infectious agent—or chronic—usually associated with other medical conditions affecting the intestine such as IBD, IBS, and Crohn's Disease. Loss of microbial balance in the gastrointestinal tract is commonly associated with all forms of diarrhea. Thus, probiotics have garnered clinical attention as potential therapeutic or preventative treatments of the disease.

Unfortunately, the evidence of probiotic efficacy in clinical settings is only modest for the prevention of diarrhea and contradictory results are common likely due to differences in populations studied, the type of probiotic, duration of treatment and dosage [Guandalini, 2011]. Additionally, many probiotics are hindered by inherent physiological and technological weaknesses and often the most clinically promising strains are not suitable as therapeutics. The most common probiotics tested for their impact on diarrhea are Lactobacillus, Bifidobacterium lactis, and Streptococcus, either alone or in combination with each other. Because of these variables, it is unlikely that the current wild type probiotics will be viable candidates for successful therapeutic interventions for diarrhea.

The treatment of gastrointestinal infections has been further complicated by the rise of antibiotic resistance. Over 70% of hospital bacterial infections harbor resistance to one or more classes of antibiotics. The prevalence of antibiotic-resistant pathogenic microbial infection stems from a confluence of practices and policies. To date, the rise of drug resistant pathogens has been addressed by improved containment practices, judicious use of antibiotics, and government-sponsored antibiotic research and development programs. Despite these efforts, the spread of antibiotic resistance continues to be a significant and growing threat.

Urinary tract infections affect 50% of women and 12% of men at least once in their lifetimes, with 80% of these infections caused by a group of Escherichia coli known as uropathogenic E. coli (UPEC) [Brumbaugh, 2012]. Similar to gastrointestinal infections, UPEC infections are often complicated by resistance to multiple antibiotics. 25% of women with urinary tract infections suffer from a recurrent infection within 6 to 12 months of the initial infection, and 3% of all women suffer from persistently recurring urinary tract infections. Prophylactic antibiotics are the current course of treatment for women with persistently recurring urinary tract infections; rising rates of antibiotic resistance are already driving physicians to abandon first- and second-line antibiotics. In addition to the complications of persistent UPEC infections, comorbidities such as secondary yeast infections and gastrointestinal infections increase the importance of developing new treatments.

Accordingly, new strategies for the treatment of skin, gastrointestinal or urinary tract disease and infection, including those stemming of drug resistant microbes, are needed. Furthermore, the ability to tailor a probiotic to target a specific pathogen or toxin would offer a novel therapy for skin, gastrointestinal or urinary tract disease and/or infection. In addition, the ability to endow the probiotic with the ability to target drug resistant microbes would be of significant therapeutic value.

Beyond the microbiome, there is also a need to decontaminate areas that harbor antibiotic resistant or otherwise pathogenic bacteria Animal feed has been identified as a source of drug resistant microbes entering the food supply [Allen, 2014]. Subtherapeutic levels of antibiotics are commonly used as animal feed additives; this practice has exacerbated the spread of antibiotic resistant microbes in agriculture and in clinical settings [Silbergeld 2008]. Engineered probiotics targeting drug resistant microbes in livestock or animal feed would be a new strategy to controlling the spread of antibiotic resistance genes in the food supply.

Strategies to treat animal feed with beneficial probiotics can likewise be adapted to the decontamination of other environments that harbor pathogenic bacteria. Probiotics can be designed to target pathogenic bacteria that have been used as biological weapons, such as Bacillus anthracis. These probiotics could be precisely targeted to select agents via topical application and/or ingestion of the probiotic, and by designing the the probiotic to target gene sequences unique to the select agent of concern. These approaches could also be adapted to the decontamination of environmental sites that were contaminated by select agent bacteria.

### SUMMARY

Systems and methods of the present disclosure provide for engineered genetic systems with many applications, such as the treatment of diseases and infections using engineered probiotics. Furthermore, systems and methods of the present disclosure can be used to reduce or eliminate antibiotic resistance, the spread of antibiotic resistance, and/or the spread of pathogenic elements, within or beyond a microbial community. In addition to engineered probiotics, other cells can also be engineered using similar methods to achieve, for example, gene editing and gene therapy.

In a certain aspect, the disclosure described herein provides a probiotic engineered to confer the ability to degrade undesirabled genes and/or genetic elements of interest from a microbial population. The engineered probiotic comprises a system to target and degrade selected gene(s) of interest, a system to facilitate the dispersal of the gene degradation system throughout a microbial community, and optionally a system to ensure the maintenance and/or containment of the engineered probiotic and/or gene degradation system without the use of antibiotics. In various embodiments, the target gene(s) of interest include genetic elements that encode virulence factors (including both colonization and fitness factors), toxins, effectors, pathogenic components and/or antibiotic resistance traits. In some aspects, the engineered probiotic may be used in either human therapeutic or veterinary applications.

In one aspect, an engineered genetic system is provided, comprising: a nuclease module designed to specifically target and degrade a nucleic acid of interest encoding a virulence factor, toxin, effector, pathogenic component and/or antibiotic resistance trait; and a synthetic mobile genetic element (MGE) module capable of dispersing the system from one host cell to another; wherein the nuclease module comprises a nuclease encoded by a gene located in the MGE module. The engineered genetic system can be used to target and degrade the nucleic acid of interest within an organism such as a bacterial cell.

In some embodiments, the nuclease module comprises a Cas protein and one or more synthetic crRNAs wherein each crRNA comprises a spacer having a target sequence derived from the nucleic acid of interest. The Cas protein can be expressed constitutively or inducibly. The Cas protein may, in one example, be expressed from SEQ ID NO:1. In one example, the Cas protein is Streptomyces pyogenes Cas9 nuclease. The crRNA(s) can be transcribed and processed from a CRISPR array which may be placed under the control of an inducible promoter or a constitutive promoter. In one example, the CRISPR array has SEQ ID NO:3. In some embodiments, the nuclease module can further include a tracrRNA that forms a complex with the Cas protein and crRNA. The tracrRNA may be placed under the control of an inducible promoter or a constitutive promoter. In one example, the tracrRNA is transcribed from SEQ ID NO:2. In certain embodiments, the tracrRNA and crRNA can be provided in a single guide RNA. In certain embodiments, the system can include multiple guide RNAs. These guide RNAs may target a single gene at multiple nucleotide positions, or they may target multiple genes of interest for degradation.

The nucleic acid of interest can be DNA or RNA. In some embodiments, the target sequence can be immediately adjacent to a Protospacer Associated Motif (PAM) in the nucleic acid of interest. When the Cas protein is Streptomyces pyogenes Cas9 nuclease, the PAM can have the NGG sequence that is 3′ of the target sequence.

In various embodiments, the nuclease can include a Transcription Activator-Like Effector Nuclease (TALEN) designed to target and degrade the nucleic acid of interest, a Zinc Finger Nuclease (ZFN) designed to target and degrade the nucleic acid of interest, and/or a meganuclease designed to target and degrade the nucleic acid of interest.

In some embodiments, the virulence factor, toxin, effector, pathogenic component and/or antibiotic resistance trait are selected from those listed in Tables 1 and 2. For example, the virulence factor can be a colonization or fitness factor.

The MGE module, in some embodiments, comprises a gene encoding a transposase and a MGE selected from a bacteriophage, conjugative plasmid, or conjugative transposon. For example, the MGE can be derived from Tn916, RK2, P1, Tn5280, or Tn4651.

In some embodiments, one or more CRISPR elements may be combined with an MGE in one plasmid to facilitate transfer between bacterial cells. The plasmid may further be designed as in SEQ ID NO:17 or SEQ ID NO:18.

In some embodiments, CRISPR elements may be combined with an MGE to facilitate transfer between bacterial cells, including a transposase that allows transfer of the CRISPR elements to the genome of the recipient cell. For example, the transposase can be derived from the Tn3 or Tn5 transposable elements. Two such designs are provided as SEQ ID NO:19 and SEQ ID NO:20.

In an examplary aspect, an engineered gene targeting and degradation system is provided. The system includes: a Cas protein; one or more synthetic crRNAs wherein each crRNA comprises a spacer having a sequence of interest derived from a target gene, wherein the target gene encodes a virulence factor, toxin, effector, pathogenic component and/or antibiotic resistance trait; optionally, a tracrRNA that forms a complex with Cas protein and crRNA; and a synthetic mobile genetic element (MGE) capable of dispersing the system between hosts.

The present disclosure also provides an engineered organism comprising the engineered genetic system disclosed herein, for use in the prevention and/or treatment of a disease or infection, the prevention and/or treatment of antibiotic resistance, limiting the spread of antibiotic resistance, and/or decontamination of emvironmental pathogens. In some embodiments, the engineered genetic system is introduced into a host selected from a bacterial cell, archaea cell and/or yeast cell.

In yet another aspect, an engineered probiotic comprising the engineered organism described herein is provided. The engineered probiotic can be an oral probiotic for use in the gastrointestinal tract, a probiotic for use in the urinary tract, and/or a topical probiotic for use on the skin.

In various embodiments, the engineered probiotic for use in the gastrointestinal tract and/or in the urinary tract can be based on a host selected from Bacteroidetes, Firmicutes, Proteobacteria, Actinobacteria, Verrucomicrobia or Fusobacteria divisions of Bacteria. For example, the host may be selected from Bacteroides species including Bacteroides AFS519, Bacteroides sp. CCUG 39913, Bacteroides sp. Smarlab 3301186, Bacteroides ovatus, Bacteroides salyersiae, Bacteroides sp. MPN isolate group 6, Bacteroides DSM 12148, Bacteroides merdae, Bacteroides distasonis, Bacteroides stercosis, Bacteroides splanchnicus, Bacteroides WH2, Bacteroides uniformis, Bacteroides WH302, Bacteroides fragilis, Bacteroides caccae, Bacteroides thetaiotamicron, Bacteroides vulgatus, and Bacteroides capillosus. The host can also be selected from Clostridium species including Clostridium leptum, Clostridium boltaea, Clostridium bartlettii, Clostridium symbiosum, Clostridium sp. DSM 6877(FS41), Clostridium A2-207, Clostridium scindens, Clostridium spiroforme, Clostridium sp. A2-183, Clostridium sp. SL6/1/1, Clostridium sp. GM2/1, Clostridium sp. A2-194, Clostridium sp. A2-166, Clostridium sp. A2-175, Clostridium sp. SR1/1, Clostridium sp. L1-83, Clostridium sp. L2-6, Clostridium sp. A2-231, Clostridium sp. A2-165 and Clostridium sp. SS2/1. The host may also be selected from Eubacterium species including Eubacterium plautii, Eubacterium ventriosum, Eubacterium halii, Eubacterium siraeum, Eubacterium eligens, and Eubacterium rectale. In some embodiments, the host is selected from Alistipes finegoldii, Alistipes putredinis, Anaerotruncus colihominis, Allisonella histaminiformans, Bulleida moorei, Peptostreptococcus sp. oral clone CK035, Anaerococcus vaginalis, Ruminococcus bromii, Anaerofustis stercorihominis, Streptococcus mitis, Ruminococcus callidus, Streptococcus parasanguinis, Coprococcus eutactus, Gemella haemolysans, Peptostreptococcus micros, Ruminococcus gnavus, Coprococcus catus, Roseburia intestinalis, Roseburia faecalis, Ruminococcus obeum, Catenibacterium mitsuokai, Ruminococcus torques, Subdoligranulum variabile, Dorea formicigenerans, Dialister sp. E2_20, Dorea longicatena, Faecalibacterium prausnitzii, Akkermansia muciniphila, Fusobacterium sp. oral clone R002, Escherichia coli, Haemophilus parainfluenziae, Bilophila wadsworthii, Desulfovibrio piger, Cornyebacterium durum, Bifidobacterium adolescentis, Actinomyces graevenitzii, Cornyebacterium sundsvallense, Actinomyces odontolyticus, and Collinsella aerofaciens. In certain embodiments, the host is selected from the genus Lactobacillus, Bifidobacterium, and/or Streptococcus. For example, the host can be selected from Lactobacillus casei, Lactobacillus lactis, Lactobacillus reuteri, Lactobacillus rhamnosus, Lactobacillus acidophilus, Lactobacillus plantarum, Lactobacillus paracasei, Lactobacillus bulgaricus, Lactobacillus fermentum and Lactobacillus johnsonii. The host may also be selected from Bacillus coagulans GBI-30, 6086, Bifidobacterium animalis subsp. lactis BB-12, Bifidobacterium longum subsp. infantis 35624, Lactobacillus paracasei Stl 1 (or NCC2461), Lactobacillus johnsonii La1 (Lactobacillus johnsonii NCC533), Lactobacillus plantarum 299v, Lactobacillus reuteri ATCC 55730, Lactobacillus reuteri DSM 17938, Lactobacillus reuteri ATCC PTA 5289, Saccharomyces boulardii, Lactobacillus rhamnosus GR-1, Lactobacillus reuteri RC-14, Lactobacillus acidophilus CL1285, Lactobacillus casei LBC80R, Lactobacillus plantarum HEAL 9, Lactobacillus paracasei 8700:2, Streptococcus thermophilus, Lactobacillus paracasei LMG P 22043, Lactobacillus johnsonii BFE 6128, Lactobacillus fermentum ME-3, Lactobacillus plantarum BFE 1685, Bifidobacterium longum BB536 and Lactobacillus rhamnosus LB21 NCIMB 40564. In one embodiment, the host is selected from an Escherichia coli strain, such as E. coli HS, E. coli SE11, E. coli SE15, E. coli W, and E. coli Nissle 1917. In various embodiments, the host may be a clinical or environmental isolate of a bacterial strain.

In some embodiments, the engineered probiotic for use on the skin can be based on a host which is selected from the genera Staphylococcus, Propionibacterium, Malassezia, Corynebacterium, Brevibacterium, Lactococcus, Lactobacillus, Micrococcus, Debaryomyces, and Cryptococcus. For example, the host may be selected from Staphylococcus epidermis, Staphylococcus saprophyticus, Propionibacterium acnes, Propionibacterium avidum, Lactococcus lactis, Lactobacillus reuteri and Lactobacillus plantarum.

In a further aspect, provided herein is a method for prevention and/or treatment of a disease or infection, for prevention and/or treatment of antibiotic resistance, and/or for limiting the spread of antibiotic resistance. The method includes administering an effective amount of the engineered probiotic described herein to a subject in need thereof. In various embodiments, the subject can be a human or an animal.

In still another aspect, the above systems and methods can be used to limit the occurrence or spread of virulence factors, pathogenic elements and/or antibiotic resistance genes in a microbial population at an environmental site. In some embodiments, the environmental site is animal feed, farm or other material or location where animals or livestock frequent. In other embodiments, the environmental site is a building or location where humans frequent, such as a hospital or other clinical settings.

In still another aspect, the above systems and methods can be used to deliver genetic systems to a mammalian (e.g., human or animal) cell. The engineered cell can include a nuclease module to target and degrade selected gene(s) of interest, and a MGE module to facilitate the dispersal of the gene degradation system throughout the population of cells. For example, a bacterial cell or a virus can be engineered to contain the nuclease module and the MGE and to invade a mammalian cell. In various embodiments, the target gene(s) of interest include genetic elements that encode a disease factor. In some embodiments, the engineered cell may be used in gene therapy.

Also provided herein is a population of cells, comprising at least one engineered organism or engineered probiotic disclosed herein, wherein the MGE module in the at least one engineered organism or probiotic is capable of spreading the engineered genetic system into other cells in the population. Overtime, the population of cells will be subject to the engineered genetic system which can target and degrade the nucleic acid of interest in the population of cells. This way, “vaccination” of a population of cells with one engineered cell or a small group of cells can effectively combat or eleminate undesirable trais of the population of cells, thereby achieving, for example, the treatment of gastrointestinal, skin or urinary tract diseases and infections, prevention of the spread of antibiotic resistance, and/or decontamination of environmental pathogens

### BRIEF DESCRIPTION OF THE FIGURES

FIG. 1 lists three equations that comprise a basic model to describe the spread of the gene targeting and degradation system from the engineered probiotic to other members of a microbial community. P(t) and F(t) denotes the subpopulations with and without the gene targeting and degradation system, respectively. R(t) denotes the pool of growth resources available. Other variables are defined in Table 11.

FIG. 2 depicts a schematic of the Yersinia pestis biovar Orientalis str IP275 chloramphenicol acetyltransferase coding sequence (CAT, Genbank accession NC_009141 40824 . . . 41483). Sites suitable for targeting with the S. pyogenes Cas9 nuclease are annotated in dark gray. Selected target sites and gene features of interest are annotated in light gray.

FIGS. 3A-3E depict exemplary designs of the three elements of a CRISPR/Cas gene targeting system: the CRISPR array transcription cassette (FIG. 3A), the tracrRNA transcription cassette (FIG. 3B), and the Cas9 expression cassette (FIG. 3C). The CRISPR array may contain one or more spacers: a one spacer design is shown in (FIG. 3A) while a five spacer design is shown in (FIG. 3D). In an alternative design, the tracrRNA and target spacer are combined into a single guide RNA or gRNA transcription cassette (FIG. 3E).

FIGS. 4A-4D show the results of challenging an engineered probiotic strain versus a control strain with a plasmid encoding an antibiotic resistance trait of interest (Yersinia pestis biovar Orientalis str IP275 chloramphenicol acetyltransferase coding sequence or CAT, SEQ ID NO:4). The challenge plasmid is also designed to encode a fluorescent protein for convenience of observation. The engineered probiotic strain encodes a CRISPR/Cas gene targeting and degradation system comprising a Cas9 expression cassette (SEQ ID NO:1), a tracrRNA (SEQ ID NO:2) and a CRISPR array (SEQ ID NO:3). The system is designed to target the CAT gene. The control strain is similar to the engineered probiotic strain but it omits the CRISPR array (SEQ ID NO:3). The engineered probiotic strain and control strain are challenged via transformation. The engineered probiotic has a reduced number of colonies after transformation with the plasmid and growth on chloramphenicol plates (FIG. 4A) relative to the control strain (FIG. 4B) indicating the efficacy of the gene targeting and degradation system. In FIG. 4C and FIG. 4D, results from a similar experiment are shown except that transformed cells were grown on kanamycin plates which selects for one (but not all) of the CRISPR components. Cell viability is not reduced, and no fluorescent colonies are visible in FIG. 4C indicating that the engineered probiotic is 100% effective in the absence of selection of the transformed plasmid.

FIG. 5 shows the results of challenging an engineered probiotic strain versus a control strain with a set of five different plasmids each encoding an antibiotic resistance trait of interest (Yersinia pestis biovar Orientalis str IP275 chloramphenicol acetyltransferase coding sequence or CAT, SEQ ID NOs:4-8). All of the challenge plasmids are also designed to encode various fluorescent proteins for convenience. The engineered probiotic strain encodes a CRISPR/Cas gene targeting and degradation system comprising a Cas9 expression cassette (SEQ ID NO:1), a tracrRNA (SEQ ID NO:2) and a CRISPR array (SEQ ID NO:3). The system is designed to target the CAT gene. The control strain is similar to the engineered probiotic strain but it omits the CRISPR array (SEQ ID NO:3). The engineered probiotic strain and control strain are challenged via transformation (top row of panels corresponds to SEQ ID NO:4-6, bottom row of panels corresponds to SEQ ID NOs:7-8). The engineered probiotic has a reduced number of colonies after transformation with the plasmid and growth on chloramphenicol plates (left in each panel) than the control strain (right in each panel). Each panel includes replicate results for each challenge experiment (top and bottom of each panel).

FIG. 6 shows the results of challenging an engineered probiotic strain versus a control strain with a plasmid that encodes an antibiotic resistance trait of interest that is not the target of the engineered probiotic. The engineered probiotic strain encodes a CRISPR/Cas gene targeting and degradation system comprising a Cas9 expression cassette (SEQ ID NO:1), a tracrRNA (SEQ ID NO:2) and a CRISPR array (SEQ ID NO:3). The system is designed to target the Yersinia pestis biovar Orientalis str IP275 chloramphenicol acetyltransferase coding sequence. The control strain is similar to the engineered probiotic strain but it omits the CRISPR array (SEQ ID NO:3). The engineered probiotic strain and control strain are challenged via transformation with a plasmid encoding a tetracycline resistance gene (SEQ ID NO:9). The engineered probiotic strain (left) and control strain (right) show no observable difference in the number of colonies after transformation with the plasmid and growth on tetracycline plates. The top half of each plate is a 1:1000 dilution of the transformation mix plated on the bottom half of each plate. The similar colony densities per unit area indicates that the engineered probiotic does not suffer from reduced cell viability or competence relative to the control strain.

FIG. 7 shows the results of challenging a target strain with various guide RNAs (gRNAs) targeted at difference sequences to test the ability of CRISPR/Cas gene targeting and degradation systems to remove preexisting undesirable genes. The target strain comprises a low copy plasmid encoding a Cas9 expression cassette (SEQ ID NO:1) and a high copy plasmid encoding a Yersinia pestis biovar Orientalis str IP275 chloramphenicol acetyltransferase coding sequence (CAT) and a fluorescent protein (SEQ ID NO:4). Each column shows challenge via transformation results from a different gRNA construct. The gRNAs in columns 1-4 (SEQ ID NO:10-13) and 6-7 (SEQ ID NO:15-16) were targeted against the CAT gene whereas the gRNA (SEQ ID NO:14) in column 5 was targeted against an off-target gene not present in the target strain. Columns 1-4 and 6-7 vary in the identity of the promoter driving transcription of the gRNA, the plasmid, and the target site of the gRNA. The top row shows results from transformants plated on apramycin and ampicillin which selected for the Cas9 and gRNA plasmids, respectively. The bottom row shows results from transformants plated on apramycin, ampicillin, and chloramphenicol. Uneven distributions of colony growth reflect variations in antibiotic concentration across the agar plate.

FIGS. 8A-8B depict schematics of exemplary designs of an engineered probiotic. FIG. 8A depicts the complete mobilizable gene targeting and degradation system including the antibiotic-free selection and containment mechanism (denoted by dark gray box and labeled marker). FIG. 8B depicts an examplary design of the selection and containment mechanism derived from the raf operon that is controlled by raffinose.

FIG. 9 depicts the performance of the device when deployed in commensal strains of E. coli, specifically selected strains from the E. coli Collection of Reference (ECOR). These strains have not undergone extensive laboratory evolution, and are therefore closely related to the E. coli found in the healthy human gut. FIG. 9 demonstrates that the device prevents uptake of CAT plasmids in this context. *Growth is characterized in two columns: the “E” column shows the expected growth phenotype based on previous experiments with laboratory strains of Escherichia coli, while the “0” column shows the observed growth phenotype in this experiment with commensal strains of Escherichia coli. In both columns, the −symbol denotes no growth under the illustrated condition, while the + symbol denotes normal growth. Photos to the right of these columns show the actual growth phenotype of these strains. From these data it is evident that the CRISPR/Cas system behaves similarly in both laboratory and commensal strains of Escherichia coli.

FIG. 10 depicts additional design schemes for selection mechanisms based on the raf operon. Also depicted is a control design that expresses the fluorescent Gemini reporter instead of the raf operon. For simplicity, the ribosome binding sites controlling the expression of rafB and rafD are not shown in this figure.

FIG. 11 illustrates the performance of the constitutive raf operon as a selection module. The Gemini column represents Escherichia coli cultures containing the Gemini control plasmid shown in FIG. 10. The Raf Operon column represents Escherichia coli cultures containing the constitutive Raf selection plasmid shown in FIG. 10. The 1:1 Mix column represents an Escherichia coli culture inoculated with equal amounts of the Gemini and Raf Operon Escherichia coli strains. The 1:4 Mix column represents an Escherichia coli culture inoculated at a ratio of 1 unit of Gemini Escherichia coli to every 4 units of Raf Operon Escherichia coli. All cultures shown were inoculated with the same sum total amount of Escherichia coli and grown in Terrific Broth overnight prior to measurement. Shading of each column illustrates the amount of raffinose applied to each culture. Lower fluorescence indicates that the Raf Operon strain is outcompeting the Gemini strain. The Raf operon provides a significant growth advantage over the Gemini strain at a raffinose concentration of 1.0%. This advantage is potentially larger than suggested by the loss of Gemini fluorescence in the 1:1 and 1:4 mixed samples, as the Gemini strain appears to grow to slightly lower densities in higher concentrations of raffinose, while simultaneously exhibiting a higher fluorescence signal at higher concentrations of raffinose.

FIG. 12 shows the performance of the constitutive Raf operon in commensal Escherichia coli strains from the ECOR collection. The assay was set up as in FIG. 11, with the ECOR strain in each column mixed at a 1:1 ratio with the laboratory Escherichia coli strain hosting the Gemini control plasmid. Both ECOR-08 and ECOR-51 display a clear growth advantage in the presence of raffinose.

FIG. 13 depicts an updated single-plasmid design for the device. The CRISPR module has been updated to be regulated by the Lac repressor, to prevent activation of the CRISPR device in the absence of lactose or lactose analogs such as Isopropyl β-D-1-thiogalactopyranoside or 5-bromo-4-chloro-3-indolyl-β-D-galactopyranoside. The Cas9 protein may optionally be fused with a fluorescent protein (“FP” in FIG. 13), such as a deep red fluorescent protein, to enable in vivo imaging of Cas9 expression. The CRISPR module is harbored in the payload region of a mobile plasmid (MGE module) capable of conjugation between bacterial cells (SEQ ID NOS:17 and 18); optionally this module includes a constitutive transposase for transfer of the payload region and contained CRISPR module into the genome of bacterial cells (SEQ ID NOS:19 and 20).

### DETAILED DESCRIPTION

The present disclosure relates to methods and systems for developing and using an engineered probiotic as therapeutic treatment for gastrointestinal, skin or urinary tract diseases and/or infections, as agent for combatting the spread of antibiotic resistance, and/or as tool for decontamination of environmental pathogens.

### DEFINITIONS

As used herein, the terms “nucleic acids,” “nucleic acid molecule” and “polynucleotide” may be used interchangeably and include both single-stranded (ss) and double-stranded (ds) RNA, DNA and RNA:DNA hybrids. As used herein the terms “nucleic acid”, “nucleic acid molecule”, “polynucleotide”, “oligonucleotide”, “oligomer” and “oligo” are used interchangeably and are intended to include, but are not limited to, a polymeric form of nucleotides that may have various lengths, including either deoxyribonucleotides or ribonucleotides, or analogs thereof. For example, oligos may be from 5 to about 200 nucleotides, from 10 to about 100 nucleotides, or from 20 to about 50 nucleotides long. However, shorter or longer oligonucleotides may be used. Oligos for use in the present disclosure can be fully designed. A nucleic acid molecule may encode a full-length polypeptide or a fragment of any length thereof, or may be non-coding.

Nucleic acids can refer to naturally-occurring or synthetic polymeric forms of nucleotides. The oligos and nucleic acid molecules of the present disclosure may be formed from naturally-occurring nucleotides, for example forming deoxyribonucleic acid (DNA) or ribonucleic acid (RNA) molecules. Alternatively, the naturally-occurring oligonucleotides may include structural modifications to alter their properties, such as in peptide nucleic acids (PNA) or in locked nucleic acids (LNA). The terms should be understood to include equivalents, analogs of either RNA or DNA made from nucleotide analogs and as applicable to the embodiment being described, single-stranded or double-stranded polynucleotides. Nucleotides useful in the disclosure include, for example, naturally-occurring nucleotides (for example, ribonucleotides or deoxyribonucleotides), or natural or synthetic modifications of nucleotides, or artificial bases. Modifications can also include phosphorothioated bases for increased stability.

Nucleic acid sequences that are “complementary” are those that are capable of base-pairing according to the standard Watson-Crick complementarity rules. As used herein, the term “complementary sequences” means nucleic acid sequences that are substantially complementary, as may be assessed by the nucleotide comparison methods and algorithms set forth below, or as defined as being capable of hybridizing to the polynucleotides that encode the protein sequences.

As used herein, the term “gene” refers to a nucleic acid that contains information necessary for expression of a polypeptide, protein, or untranslated RNA (e.g., rRNA, tRNA, anti-sense RNA). When the gene encodes a protein, it includes the promoter and the structural gene open reading frame sequence (ORF), as well as other sequences involved in expression of the protein. When the gene encodes an untranslated RNA, it includes the promoter and the nucleic acid that encodes the untranslated RNA.

As used herein, the term “genome” refers to the whole hereditary information of an organism that is encoded in the DNA (or RNA for certain viral species) including both coding and non-coding sequences. In various embodiments, the term may include the chromosomal DNA of an organism and/or DNA that is contained in an organelle such as, for example, the mitochondria or chloroplasts and/or extrachromosomal plasmid and/or artificial chromosome. A “native gene” or “endogenous gene” refers to a gene that is native to the host cell with its own regulatory sequences whereas an “exogenous gene” or “heterologous gene” refers to any gene that is not a native gene, comprising regulatory and/or coding sequences that are not native to the host cell. In some embodiments, a heterologous gene may comprise mutated sequences or part of regulatory and/or coding sequences. In some embodiments, the regulatory sequences may be heterologous or homologous to a gene of interest. A heterologous regulatory sequence does not function in nature to regulate the same gene(s) it is regulating in the transformed host cell. “Coding sequence” refers to a DNA sequence coding for a specific amino acid sequence. As used herein, “regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, ribosome binding sites, translation leader sequences, RNA processing site, effector (e.g., activator, repressor) binding sites, stem-loop structures, and so on.

As described herein, a genetic element may be any coding or non-coding nucleic acid sequence. In some embodiments, a genetic element is a nucleic acid that codes for an amino acid, a peptide or a protein. Genetic elements may be operons, genes, gene fragments, promoters, exons, introns, regulatory sequences, or any combination thereof. Genetic elements can be as short as one or a few codons or may be longer including functional components (e.g., encoding proteins) and/or regulatory components. In some embodiments, a genetic element includes an entire open reading frame of a protein, or the entire open reading frame and one or more (or all) regulatory sequences associated therewith. One skilled in the art would appreciate that the genetic elements can be viewed as modular genetic elements or genetic modules. For example, a genetic module can comprise a regulatory sequence or a promoter or a coding sequence or any combination thereof. In some embodiments, the genetic element includes at least two different genetic modules and at least two recombination sites. In eukaryotes, the genetic element can comprise at least three modules. For example, a genetic module can be a regulator sequence or a promoter, a coding sequence, and a polyadenlylation tail or any combination thereof. In addition to the promoter and the coding sequences, the nucleic acid sequence may comprises control modules including, but not limited to a leader, a signal sequence and a transcription terminator. The leader sequence is a non-translated region operably linked to the 5′ terminus of the coding nucleic acid sequence. The signal peptide sequence codes for an amino acid sequence linked to the amino terminus of the polypeptide which directs the polypeptide into the cell's secretion pathway.

As generally understood, a codon is a series of three nucleotides (triplets) that encodes a specific amino acid residue in a polypeptide chain or for the termination of translation (stop codons). There are 64 different codons (61 codons encoding for amino acids plus 3 stop codons) but only 20 different translated amino acids. The overabundance in the number of codons allows many amino acids to be encoded by more than one codon. Different organisms (and organelles) often show particular preferences or biases for one of the several codons that encode the same amino acid. The relative frequency of codon usage thus varies depending on the organism and organelle. In some instances, when expressing a heterologous gene in a host organism, it is desirable to modify the gene sequence so as to adapt to the codons used and codon usage frequency in the host. In particular, for reliable expression of heterologous genes it may be preferred to use codons that correlate with the host's tRNA level, especially the tRNA's that remain charged during starvation. In addition, codons having rare cognate tRNA's may affect protein folding and translation rate, and thus, may also be used. Genes designed in accordance with codon usage bias and relative tRNA abundance of the host are often referred to as being “optimized” for codon usage, which has been shown to increase expression level. Optimal codons also help to achieve faster translation rates and high accuracy. In general, codon optimization involves silent mutations that do not result in a change to the amino acid sequence of a protein.

Genetic elements or genetic modules may derive from the genome of natural organisms or from synthetic polynucleotides or from a combination thereof. In some embodiments, the genetic elements modules derive from different organisms. Genetic elements or modules useful for the methods described herein may be obtained from a variety of sources such as, for example, DNA libraries, BAC (bacterial artificial chromosome) libraries, de novo chemical synthesis, commercial gene synthesis or excision and modification of a genomic segment. The sequences obtained from such sources may then be modified using standard molecular biology and/or recombinant DNA technology to produce polynucleotide constructs having desired modifications for reintroduction into, or construction of, a large product nucleic acid, including a modified, partially synthetic or fully synthetic genome. Exemplary methods for modification of polynucleotide sequences obtained from a genome or library include, for example, site directed mutagenesis; PCR mutagenesis; inserting, deleting or swapping portions of a sequence using restriction enzymes optionally in combination with ligation; in vitro or in vivo homologous recombination; and site-specific recombination; or various combinations thereof. In other embodiments, the genetic sequences useful in accordance with the methods described herein may be synthetic oligonucleotides or polynucleotides. Synthetic oligonucleotides or polynucleotides may be produced using a variety of methods known in the art.

In some embodiments, genetic elements share less than 99%, less than 95%, less than 90%, less than 80%, less than 70% sequence identity with a native or natural nucleic acid sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences. Other techniques for alignment are described [Doolittle, 1996]. Preferably, an alignment program that permits gaps in the sequence is utilized to align the sequences. The Smith-Waterman is one type of algorithm that permits gaps in sequence alignments [Shpaer, 1997]. Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer.

As used herein, the phrase “homologous recombination” refers to the process in which nucleic acid molecules with similar nucleotide sequences associate and exchange nucleotide strands. A nucleotide sequence of a first nucleic acid molecule that is effective for engaging in homologous recombination at a predefined position of a second nucleic acid molecule can therefore have a nucleotide sequence that facilitates the exchange of nucleotide strands between the first nucleic acid molecule and a defined position of the second nucleic acid molecule. Thus, the first nucleic acid can generally have a nucleotide sequence that is sufficiently complementary to a portion of the second nucleic acid molecule to promote nucleotide base pairing. Homologous recombination requires homologous sequences in the two recombining partner nucleic acids but does not require any specific sequences. Homologous recombination can be used to introduce a heterologous nucleic acid and/or mutations into the host genome. Such systems typically rely on sequence flanking the heterologous nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

It should be appreciated that the nucleic acid sequence of interest or the gene of interest may be derived from the genome of natural organisms. In some embodiments, genes of interest may be excised from the genome of a natural organism or from the host genome, for example E. coli. It has been shown that it is possible to excise large genomic fragments by in vitro enzymatic excision and in vivo excision and amplification. For example, the FLP/FRT site specific recombination system and the Cre/loxP site specific recombination systems have been efficiently used for excision large genomic fragments for the purpose of sequencing [Yoon, 1998]. In some embodiments, excision and amplification techniques can be used to facilitate artificial genome or chromosome assembly. In some embodiments, the excised genomic fragments can be assembled with engineered promoters and/or other gene expression elements and inserted into the genome of the host cell.

As used herein, the term “polypeptide” refers to a sequence of contiguous amino acids of any length. The terms “peptide,” “oligopeptide,” “protein” or “enzyme” may be used interchangeably herein with the term “polypeptide”. In certain instances, “enzyme” refers to a protein having catalytic activities.

As used herein, unless otherwise stated, the term “transcription” refers to the synthesis of RNA from a DNA template; the term “translation” refers to the synthesis of a polypeptide from an mRNA template. Translation in general is regulated by the sequence and structure of the 5′ untranslated region (5′-UTR) of the mRNA transcript. One regulatory sequence is the ribosome binding site (RBS), which promotes efficient and accurate translation of mRNA. The prokaryotic RBS is the Shine-Dalgarno sequence, a purine-rich sequence of 5′-UTR that is complementary to the UCCU core sequence of the 3′-end of 16S rRNA (located within the 30S small ribosomal subunit). Various Shine-Dalgarno sequences have been found in prokaryotic mRNAs and generally lie about 10 nucleotides upstream from the AUG start codon. Activity of a RBS can be influenced by the length and nucleotide composition of the spacer separating the RBS and the initiator AUG. In eukaryotes, the Kozak sequence lies within a short 5′ untranslated region and directs translation of mRNA. An mRNA lacking the Kozak consensus sequence may also be translated efficiently in an in vitro systems if it possesses a moderately long 5′-UTR that lacks stable secondary structure. While E. coli ribosome preferentially recognizes the Shine-Dalgarno sequence, eukaryotic ribosomes (such as those found in retic lysate) can efficiently use either the Shine-Dalgarno or the Kozak ribosomal binding sites.

As used herein, the terms “promoter,” “promoter element,” or “promoter sequence” refer to a DNA sequence which when ligated to a nucleotide sequence of interest is capable of controlling the transcription of the nucleotide sequence of interest into mRNA. A promoter is typically, though not necessarily, located 5′ (i.e., upstream) of a nucleotide sequence of interest whose transcription into mRNA it controls, and provides a site for specific binding by RNA polymerase and other transcription factors for initiation of transcription. A promoter may be constitutively active (“constitutive promoter”) or be controlled by other factors such as a chemical, heat or light. The activity of an “inducible promoter” is induced by the presence or absence or biotic or abiotic factors. Aspects of the disclosure relate to an “autoinducible” or “autoinduction” system where an inducible promoter is used, but addition of exogenous inducer is not required. Commonly used constitutive promoters include CMV, EF1a, SV40, PGK1, Ubc, human beta actin, CAG, Ac5, Polyhedrin, TEF1, GDS, ADH1 (repressed by ethanol), CaMV35S, Ubi, H1, U6, T7 (requires T7 RNA polymerase), and SP6 (requires SP6 RNA polymerase). Common inducible promoters include TRE (inducible by Tetracycline or its derivatives; repressible by TetR repressor), GAL1 & GAL10 (inducible with galactose; repressible with glucose), lac (constitutive in the absence of lac repressor (LacI); can be induced by IPTG or lactose), T7lac (hybrid of T7 and lac; requires T7 RNA polymerase which is also controlled by lac operator; can be induced by TRIG or lactose), araBAD (inducible by arabinose which binds repressor AraC to switch it to activate transcription; repressed catabolite repression in the presence of glucose via the CAP binding site or by competitive binding of the anti-inducer fucose), trp (repressible by tryptophan upon binding with TrpR repressor), tac (hybrid of lac and trp; regulated like the lac promoter; e.g., tad and tacII), and pL (temperature regulated). The promoter can be prokaryotic or eukaryotic promoter, depending on the host. Common promoters and their sequences are well known in the art.

One should appreciate that promoters have modular architecture and that the modular architecture may be altered. Bacterial promoters typically include a core promoter element and additional promoter elements. The core promoter refers to the minimal portion of the promoter required to initiate transcription. A core promoter includes a Transcription Start Site, a binding site for RNA polymerases and general transcription factor binding sites. The “transcription start site” refers to the first nucleotide to be transcribed and is designated +1. Nucleotides downstream of the start site are numbered +1, +2, etc., and nucleotides upstream of the start site are numbered −1, −2, etc. Additional promoter elements are located 5′ (i.e., typically 30-250 bp upstream of the start site) of the core promoter and regulate the frequency of the transcription. The proximal promoter elements and the distal promoter elements constitute specific transcription factor site. In prokaryotes, a core promoter usually includes two consensus sequences, a −10 sequence or a −35 sequence, which are recognized by sigma factors. The −10 sequence (10 bp upstream from the first transcribed nucleotide) is typically about 6 nucleotides in length and is typically made up of the nucleotides adenosine and thymidine (also known as the Pribnow box). The presence of this box is essential to the start of the transcription. The −35 sequence of a core promoter is typically about 6 nucleotides in length. The nucleotide sequence of the −35 sequence is typically made up of the each of the four nucleosides. The presence of this sequence allows a very high transcription rate. In some embodiments, the −10 and the −35 sequences are spaced by about 17 nucleotides. Eukaryotic promoters are more diverse than prokaryotic promoters and may be located several kilobases upstream of the transcription starting site. Some eukaryotic promoters contain a TATA box, which is located typically within 40 to 120 bases of the transcriptional start site. One or more upstream activation sequences (UAS), which are recognized by specific binding proteins can act as activators of the transcription. Theses UAS sequences are typically found upstream of the transcription initiation site. The distance between the UAS sequences and the TATA box is highly variable and may be up to 1 kb.

As used herein, the term “vector” refers to any genetic element, such as a plasmid, phage, transposon, cosmid, chromosome, artificial chromosome, episome, virus, virion, etc., capable of replication when associated with the proper control elements and which can transfer gene sequences into or between cells. The vector may contain a selection module suitable for use in the identification of transformed or transfected cells. For example, selection modules may provide antibiotic resistant, fluorescent, enzymatic, as well as other traits. As a second example, selection modules may complement auxotrophic deficiencies or supply critical nutrients not in the culture media. Types of vectors include cloning and expression vectors. As used herein, the term “cloning vector” refers to a plasmid or phage DNA or other DNA sequence which is able to replicate autonomously in a host cell and which is characterized by one or a small number of restriction endonuclease recognition sites and/or sites for site-specific recombination. A foreign DNA fragment may be spliced into the vector at these sites in order to bring about the replication and cloning of the fragment. The term “expression vector” refers to a vector which is capable of expressing of a gene that has been cloned into it. Such expression can occur after transformation into a host cell, or in IVPS systems. The cloned DNA is usually operably linked to one or more regulatory sequences, such as promoters, activator/repressor binding sites, terminators, enhancers and the like. The promoter sequences can be constitutive, inducible and/or repressible.

As used herein, the term “host” or “host cell” refers to any prokaryotic or eukaryotic single cell (e.g., yeast, bacterial, archaeal, etc.) cell or organism. The host cell can be a recipient of a replicable expression vector, cloning vector or any heterologous nucleic acid molecule. Host cells may be prokaryotic cells such as species of the genus Escherichia or Lactobacillus, or eukaryotic single cell organism such as yeast. The heterologous nucleic acid molecule may contain, but is not limited to, a sequence of interest, a transcriptional regulatory sequence (such as a promoter, enhancer, repressor, and the like) and/or an origin of replication. As used herein, the terms “host,” “host cell,” “recombinant host” and “recombinant host cell” may be used interchangeably. For examples of such hosts, see [Sambrook, 2001].

One or more nucleic acid sequences can be targeted for delivery to target prokaryotic or eukaryotic cells via conventional transformation techniques. As used herein, the term “transformation” is intended to refer to a variety of art-recognized techniques for introducing an exogenous nucleic acid sequence (e.g., DNA) into a target cell, including calcium phosphate or calcium chloride co-precipitation, conjugation, electroporation, sonoporation, optoporation, injection and the like. Suitable transformation media include, but are not limited to, water, CaCl₂, cationic polymers, lipids, and the like. Suitable materials and methods for transforming target cells can be found in [Sambrook, 2001], and other laboratory manuals.

As used herein, the term “selection module” or “reporter” refers to a gene, operon, or protein that can be attached to a regulatory sequence of another gene or protein of interest, so that upon expression in a host cell or organism, the reporter can confer certain characteristics that can be relatively easily selected, identified and/or measured. Reporter genes are often used as an indication of whether a certain gene has been introduced into or expressed in the host cell or organism. Examples of commonly used reporters include: antibiotic resistance genes, fluorescent proteins, auxotropic selection modules, β-galactosidase (encoded by the bacterial gene lacZ), luciferase (from lightning bugs), chloramphenicol acetyltransferase (CAT; from bacteria), GUS (β-glucuronidase; commonly used in plants) and green fluorescent protein (GFP; from jelly fish). Reporters or selection moduless can be selectable or screenable. A selection module (e.g., antibiotic resistance gene, auxotropic gene) is a gene confers a trait suitable for artificial selection; typically host cells expressing the selectable selection module is protected from a selective agent that is toxic or inhibitory to cell growth. A screenable selection module (e.g., gfp, lacZ) generally allows researchers to distinguish between wanted cells (expressing the selection module) and unwanted cells (not expressing the selection module or expressing at insufficient level).

The term “virulence factor”, “toxin”, “effector” or “pathogenic component” as used herein, refers to molecules that enable otherwise commensal organisms to cause disease or otherwise disrupt a microbial community. The removal of these factors or genetic elements encoding them (including both DNA and RNA) from a commensal bacterial geneome, or the loss of a plasmid or other mobile genetic element encoding them from a commensal bacterial genome is understood to restore the host bacteria to a non-pathogenic state. These factors include but are not limited to any molecule that enables a pathogen to colonize a niche in the host, evade the host's immune system, inhibit the host's immune response, damage the host, enter or exit out of cells, or obtain nutrition from the host. For example, one type of such factor is colonization factors that help the establishment of the pathogen at the appropriate portal of entry. Pathogens usually colonize host tissues that are in contact with the external environment. Sites of entry in human hosts include the urogenital tract, the digestive tract, the respiratory tract and the conjunctiva. Organisms that infect these regions have usually developed tissue adherence mechanisms and some ability to overcome or withstand the constant pressure of the host defenses at the surface, and factors involved therewith have been identified as colonization factors.

The term “pathogenic element”, as used herein, refers to genetic elements (including both DNA and RNA) that enable otherwise commensal organisms to cause disease or otherwise disrupt a microbial community. The removal of pathogenic elements from a commensal bacterial geneome, or the loss of a plasmid or other mobile genetic element propagating pathogenic elements from a commensal bacterial genome is understood to restore the host bacteria to a non-pathogenic state. Pathogenic elements include but are not limited to pathogenicity islands. Pathogenic elements (including both DNA and RNA) may encode virulence factors, toxins, effectors or pathogenic components.

The term “mobile genetic element” or “MGE” refers to genetic elements that encode enzymes and other proteins transposase) that mediate the movement of DNA within genomes (intracellular mobility) or between cells (intercellular mobility). Examples include transposons, plasmids, bacteriophage, and pathogenicity islands. The MGE can be naturally occurring or engineered. The MGE can be cell-type specific, tissue specific, organism specific, or species specific (e.g., bacteria specific or human specific). The MGE can also be non-specific with respect to cell-type, tissue, organism and/or species.

The term “engineer,” “engineering” or “engineered,” as used herein, refers to genetic manipulation or modification of biomolecules such as DNA, RNA and/or protein, or like technique commonly known in the biotechnology art.

The locus tags and accession numbers provided throughout this description are derived from the NCBI database (National Ceter for Biotechnology Information) maintained by the National Institute of Health, USA. The accession numbers are provided in the database on Jan. 16, 2014.

Other terms used in the fields of recombinant nucleic acid technology, microbiology, metabolic engineering, and molecular and cell biology as used herein will be generally understood by one of ordinary skill in the applicable arts.

### Target Genes for Degradation

In one aspect, the present disclosure provides for genes of interest that constitute preferred targets for degradation by the engineered probiotic. Many microbial species have strains that exist as commensals as part of the natural, healthy microbial flora as well as pathogenic and/or virulent strains capable of causing disease. For example, Escherichia coli exists in the human gut as a commensal organism but pathogenic strains are also known. Major categories of E. coli pathogens include enteropathogenic E. coli (EPEC), enterohemorrhagic E. coli (EHEC), enterotoxigenic E. coli (ETEC), enteroaggregative E. coli (EAEC), enteroinvasive E. coli (EIEC), diffusely adherent E. coli (DAEC), enteroaggregative E. coli ST (EAST) [Kaper, 2004]. Other categories of E. coli pathogens are known to be extraintestinal (ExPEC) including uropathogenic E. coli (UPEC) and meningitis-associated E. coli (MNEC). Despite these varied mechanisms of pathogenesis, each of these diseases are caused by strains that are largely similar to commensal E. coli; these strains are differentiated by a small number of specific virulence attributes responsible for each disease. Genetic elements that encode these virulence attributes are frequently found on mobilizable elements that can be readily transferred into new strains to create new virulence factor combinations. Thus, it is these genetic elements themselves, rather than a particular strain or species, that is the basic unit of selection and evolution in a microbial population. In some embodiments, genes that encode virulence attributes are gene targets for the engineered probiotic of this disclosure. Exemplary virulence factors (including both colonization and fitness factors), toxins and effectors are set forth below in Table 1. Note that many of the listed factors and toxins have multiple variants and/or types. A similar set of genes encoding virulence attributes may be compiled for other microbial species that include pathogenic strains.

TABLE 1
Virulence factors, toxins and effectors in pathogenic E. coli strains

| | Strain | |
|---|---|---|
| Factor | category | Activity/effect |
| IcsA (VirG) | EIEC | Nucleation of actin filaments |
| Intimin | EPEC, | Adhesin, induces T_{H}1 response |
| | EHEC | |
| Dr adhesins | DAEC, | Adhesin, binds to decay-accelerating factor (DAF), activates |
| | UPEC | phosphatidylinositol 3-kinase (PI-3), induces MHC class I |
| | | chain-related gene A (MICA) |
| P (Pap) fimbriae | UPEC | Adhesin; induces cytokine expression |
| CFAs, CS, or PCF | ETEC | Adhesin; colonization factor antigens, coli surface antigens, or |
| | | putative colonization factors |
| Type-1 fimbrae | All | UPEC adhesion; binds to uroplakin |
| F1C fimbriae | UPEC | Adhesin |
| S fimbriae | UPEC, | Adhesin |
| | MNEC | |
| Bundle-forming pilius (BFP) | EPEC | Type IV pilus |
| Aggregative adherence | EAEC | Adhesin |
| fimbriae | | |
| Paa | EPEC, | Adhesin |
| | EHEC | |
| ToxB | EHEC | Adhesin |
| Efa-1/LifA | EHEC | Adhesin |
| Long polar fimbriae (LPF) | EHEC, | Adhesin |
| | EPEC | |
| Saa | EHEC | Adhesin |
| OmpA | MNEC, | Adhesin |
| | EHEC | |
| Curli | Various | Adhesin; binds to fibronectin |
| IbeA, B, C | MNEC | Promotes invasion |
| AslA | MNEC | Promotes invasion |
| Dispersin | EAEC | Promotes colonization; aids mucous penetration |
| K antigen capsules | MNEC | Antiphagocytic |
| Aerobactin | EIEC | Iron acquisition; siderophore |
| Yersiniabactin | Various | Iron acquisition; siderophore |
| IreA | UPEC | Iron acquisition; siderophore receptor |
| IroN | UPEC | Iron acquisition; siderophore receptor |
| Chu (Shu) | EIEC, | Iron acquisition; haem transport |
| | UPEC, | |
| | MNEC | |
| Flagellin | All | Motility; induces cytokine expression through toll-like receptor |
| | | TLR5 |
| Lipopolysaccharide | All | Induces cytokine expression through TLR4 |
| Heat-labile enterotoxin (LT) | ETEC | ADP ribosylates and activates adenylate cyclase resulting in ion |
| | | secretion |
| Shiga toxin (Stx) | EHEC | Depurinates rRNA, inhibiting protein synthesis; induces |
| | | apoptosis |
| Cytolethal distending toxin | Various | DNaseI activity, blocks mitosis in G2/M phase |
| (CDT) | | |
| Shigella enterotoxin 1 | EAEC, | Ion secretion |
| (ShET1) | EIEC? | |
| Urease | EHEC | Cleaves urea to ammonia and carbon dioxide |
| EspC | EPEC | Serine protease; ion secretion |
| EspP | EHEC | Serine protease; cleaves coagulation factor V |
| Haemoglobin-binding | ExPEC, | Degrades haemoglobin to release haem/iron |
| protease (Tsh) | APEC | |
| Pet | EAEC | Serine protease; ion secretion; cytotoxicity |
| Pic | UPEC, | Protease, mucinase |
| | EAEC, | |
| | EIEC? | |
| Sat | UPEC | Vacuolation |
| SepA | EIEC? | Serine protease |
| SigA | EIEC? | Ion secretion |
| Cycle-inhibiting factor (Cif) | EPEC, | Blocks mitosis in G2/M phase; results in inactivation of Cdk1 |
| | EHEC | |
| EspF | EPEC, | Opens tight junctions, induces apoptosis |
| | EHEC | |
| EspH | EPEC, | Modulates filopodia and pedestal formation |
| | EHEC | |
| Map | EPEC, | Stimulates Cdc42-dependent filopodia formation; disrupts |
| | EHEC | mitochondrial membrane potential |
| Tir | EPEC, | Nucleation of cytoskeletal proteins, loss of microvilli, |
| | EHEC | GTPase-activating protein (GAP)-like activity |
| IpaA | EIEC | Actin depolymerization |
| IpaB | EIEC | Apoptosis, interleukin-1 (IL-1) release; membrane insertion |
| IpaC | EIEC | Actin polymerization, activation of Cdc42 and Rac |
| IpaH | EIEC | Modulates inflammation (?) |
| IpgD | EIEC | Inositol 4-phophatase, membrane blebbing |
| VirA | EIEC | Microtubule destabilization, membrane ruffling |
| StcE | EHEC | Cleaves C1-esterase inhibitor (C1-INH), disrupts complement |
| | | cascade |
| HlyA | UPEC | Cell lysis |
| Ehx | EHEC | Cell lysis |
| Cytotoxic necrotizing factors | MNEC, | Altered cytoskeleton, multinucleation with cell enlargement, |
| (CNF1, CNF2) | UPEC, | necrosis |
| | NTEC | |
| LifA/Efa | EPEC, | Inhibits lymphocyte activation, adhesion |
| | EHEC | |
| Shigella enterotoxin 2 | EIEC, ETEC | Ion secretion |
| (ShET2) | | |
| Heat-stable enterotoxin a | ETEC | Activates guanylate cyclase resulting in ion secretion |
| (STa) | | |
| Heat-stable enterotoxin b | ETEC | Increase intracellular calcium resulting in ion secretion |
| (STb) | | |
| EAST | Various | Activates guanylate cyclase resulting in ion secretion |

Exemplary target genes for degradation associated with skin disease and infection include genes encoding toxic shock syndrome toxin-1 (TSST-1, Accession J02615) and staphylococcal superantigen-like protein 11 (SSL11, Accession CP001996 470022 . . . 470615). Other virulence factors include staphylococcal enterotoxins such as enterotoxin type G2 (seg2), enterotoxin K (sek), enterotoxin H (seh), enterotoxin type C4 (sec4), enterotoxin L (sel), and enterotoxin A (sea); virulence genes encoded by open reading frames SAV0811, SAV1159, SAV1208, SAV1481, SAV2371, SAV2569, SAV2638, SAV0665, SAV0149, SAV0164, SAV0815, SAV1324, SAV1811, SAV1813, SAV1046, SAV0320, SAV2035, SAV0919, SAV2170, SAV1948, SAV2008, SAV1819, SAV0422, SAV0423, SAV0424, SAV0428, SAV2039, SAV1884, SAV0661 from Staphylococcus aureus subsp. Mu50 (Accession BA000017.4). Antibiotic resistance genes common to skin infections include the methicillin resistance gene PBP gene for beta-lactam-inducible penicillin-binding protein (mecA, Accession Y00688). Homologs of the listed genes offer additional target genes for degradation.

In many cases, the mechanism of antibiotic resistance is encoded by either a single or small number of genes. Similar to genes encoding virulence attributes, genetic elements that encode antibiotic resistance trait often spread through a mixed species microbial population through horizontal gene transfer. Many genes that confer clinically-relevant antibiotic resistance phenotypes to their host cell have been identified previously. In some embodiments, antibiotic resistance genes constitute gene targets for the engineered probiotic of this disclosure. Exemplary antibiotic resistance genes are set forth below in Table 2.

TABLE 2
Genes encoding antibiotic resistance

| | Resistance | | Example Locus tag/ |
|---|---|---|---|
| Antibiotic | Gene(s) | Species | Accession number |
| aminoglycosides | aadA2 | Escherichia coli | pUMNK88_133 |
| aminoglycosides | aadA | E. coli, Yersinia pestis, | pAR060302_132 |
| | | Salmonella enterica | |
| aminoglycosides | aacC | E. coli, S. enterica | pAR060302_133 |
| aminoglycosides | aacA1 | Cornyebacterium resistens | pJA144188_p28 |
| aminoglycosides | aphA | Y. pestis | YpIP275_pIP1202_0052 |
| aminoglycosides | strAB | E. coli, Yersinia ruckeri, Y. pestis, | pAR060302_44 and |
| | | S. enterica | pAR060302_43 |
| beta-lactams | pbp1A | C. resistens | CP002857.1 |
| | | | (2543105 . . . 2545177) |
| beta-lactams | pbp1B | C. resistens | CP002857.1 |
| | | | (2410761 . . . 2413031) |
| beta-lactams | pbp2A | C. resistens | CP002857.1 (48881 . . . 50284) |
| beta-lactams | pbp2B | C. resistens | CP002857.1 |
| | | | (311405 . . . 313186) |
| beta-lactams | pbp2C | C. resistens | CP002857.1 |
| | | | (1512744 . . . 1514591) |
| beta-lactams | dac | C. resistens | CP002857.1 |
| | | | (217296 . . . 218633) |
| beta-lactams | bla_{CMY-2} | E. coli, S. enterica | pAR060302_81 |
| chloramphenicol/florfenicol | floR | E. coli, S. enterica | pAR060302_39 |
| chloramphenicol | cmlA | E. coli | pUMNK88_132 |
| chloramphenicol | cat | Enterococcus faecalis, Y. pestis | YpIP275_pIP1202_0063 |
| chloramphenicol | cmx | C. resistens | pJA144188_p21 |
| erythromycin | ermA | E. faecalis | AF507977.1 (12938 . . . 13675) |
| fluoroquinolones | gyrA | C. resistens | CP002857.1 (8935 . . . 11376) |
| macrolide | mph2 and mel | E. coli | pPG010208_34 and |
| | | | pPG010208_35 |
| macrolides and lincosamides | erm(x) | C. resistens | pJA144188_p06 |
| methicillin | mecA | Staphylococcus aureus | Y00688 or KC243783.1 |
| streptomycin and | aadA1a | C. resistens | pJA144188_p26 |
| spectinomycin | | | |
| sulfonamides | sul1 | E. coli, Y. pestis, S. enterica | pAR060302_139 |
| sulfonamides | sul2 | E. coli, Y. ruckeri, Y. pestis, S. enterica | pAR060302_46 |
| tetracycline | tetA | E. coli, Y. ruckeri, Y. pestis, S. enterica | pAR060302_41 |
| tetracyclines | tet(W) | C. resistens | pJA144188_p07 |
| | bla_{SHV-1} | Y. pestis | YpIP275_pIP1202_0175 |
| trimethoprim | dhfr | Y. ruckeri | YR71pYR1_0114 |
| vancomycin/teicoplanin | van(A) | Enterococcus faecium | AM296544.1 (8898 . . . 15523) |
| vancomycin | van(B) | E. faecalis | AB374546.1 (32627 . . . 39057) |
| all antibiotic options in | bla_{NDM-1} | E. coli, Acinetobacter | HQ857107.1 (2193 . . . 3005) |
| humans | | baumanii, Klebsiella | |
| | | pneumoniae | |

Other undesirable and/or malicious genes and/or genetic elements can also be targeted. For example, in a disease caused by a genetic abnormality such as cancer, such genetic abnormality can be targeted for degradation. As a result, gene therapy can be achieved. The targeting can be cell-type specific or tissue specific (e.g., by using cell-type specific or tissue specific MGE), so as to limit to gene degradation a desired cell type or tissue.

### Targeting and Degradation of Undesirable Genes

In some embodiments, targeting and degradation of undesirable genes can be mediated by CRISPR—an acronym for clustered, regularly interspaced short palindromic repeats. CRISPRs were first described in 1987 [Ishino, 1987]. CRISPRs play a functional role in phage defense in prokaryotes [Barrangou, 2007; Horvath, 2008; Deveau, 2008]. Briefly, CRISPRs work as follows. When exposed to a phage infection or invasive genetic element, some members of the bacterial population incorporate short sequences from the foreign DNA (“spacers”) between repeated sequences within the CRISPR locus. The combined unit of repeats and spacers in tandem is referred to as the “CRISPR array.” The CRISPR array is transcribed and then processed into short crRNAs (CRISPR RNAs) each containing a single spacer and flanking repeated sequences. Spacers are derived from foreign DNA (which contains corresponding protospacers that can base pair with the spacers) and are generally stably inherited by daughter cells such that when later exposed to a phage or invasive DNA element with the same sequence, the strain is resistant to infection. CRISPRs are known to operate in conjunction with cognate Cas (CRISPR associated) protein(s) that show specificity to the repeat sequences separating the spacers [Heidelberg, 2009; Horvath, 2009; Kunin, 2007]. The Cas protein(s) operate in conjunction with the crRNA to mediate the cleavage of incoming foreign DNA where the crRNA forms an effector complex with the Cas proteins and guides the complex to the foreign DNA, which is then cleaved by the Cas proteins [Bhaya, 2011]. There are several pathways of CRISPR activation, one of which requires a tracrRNA (trans-activating crRNA, also transcribed from the CRISPR array) which plays a role in the maturation of crRNA. Then a crRNA/tracrRNA hybrid forms and acts as a guide for the Cas9 to the foreign DNA [Deltcheva, 2011]. There are also other pathways that do not require tracrRNA.

Synthetic biologists have recently demonstrated that CRISPR-Cas nucleases and associated RNAs can be repurposed to edit the genomes in bacteria, yeast and human cells [DiCarlo, 2013; Jiang, 2013; Cong, 2013; Mali 2013; Jinek, 2013]. These techniques all rely on the use of a Cas9 nuclease to introduce double strand breaks at specific loci. Since the binding specificity of Cas9 is defined by a separate RNA molecule, crRNA, Cas9 can be directed to recognize and cleave nearly all 20-30 base pair sequences. The short sequence requirements for CRISPR targeting allow Cas9 to be re-targeted simply by inserting oligonucleotides of interest into the cognate CRISPR constructs.

In some aspects, the present disclosure provides for a probiotic engineered to include a heterologous, genetic system designed to target gene(s) of interest for degradation. In some embodiments, the heterologous genetic system encodes a synthetic CRISPR-Cas device designed to target a Cas nuclease to one or more gene(s) of interest. The heterologous genetic system comprises a gene encoding a Cas nuclease, a CRISPR array containing one or more spacers derived from the target DNA flanked by CRISPR direct repeats that is transcribed and processed into one or more crRNAs, and optionally, a tracrRNA that forms a complex with the Cas protein and the crRNA. By targeting a Cas nuclease to sequence(s) within target gene(s) of interest (protospacers), the gene(s) of interest may be targeted for cleavage and therefore subsequent degradation thereof in the cell.

Viable target sequences for CRISPR/Cas systems are determined based on the specific Cas nuclease chosen; the sequence of interest (protospacer) must be immediately adjacent to a “Protospacer Associated Motif” (PAM) [Jinek, 2012]. In some embodiments, the Streptococcus pyogenes Cas9 nuclease may be used [Jiang, 2013; Cong, 2013; Mali, 2013; Jinek, 2013; Jinek, 2012]. S. pyogenes Cas9 requires the PAM “NGG” to be 3′ of the sequence of interest, where “N” can be any nucleotide. The “NGG” motif is very common in nucleic acid sequences and thus allows us to select essentially any gene of interest as a target for the engineered probiotic.

CRISPR arrays are highly repetitive due to the requirement for direct repeat sequences adjacent to spacer sequence(s). As such, CRISPR arrays can be unstable due to possible recombination events [Jiang, 2013]. To obviate this problem, it has been shown that the tracrRNA and crRNA may be combined into a single RNA sequence (“guide RNA”) that mimics the processed crRNA-tracrRNA complex. Guide RNA based designs have been demonstrated to be effective when employed for genome editing in a variety of hosts [DiCarlo, 2013; Cong, 2013; Mali, 2013]. Thus, in some embodiments, the CRISPR/Cas system of the engineered probiotic includes one or more synthetic guide RNA(s) designed to target the gene(s) of interest for degradation.

In addition to CRISPR/Cas systems, alternative nucleases may be used to target genes of interest for degradation. For example, Transcription Activator-Like Effector Nucleases (TALENs) are modular protein nucleases that can be designed to bind and cut specific DNA sequences [Cermak, 2011; Ting, 2011]. Exemplary TALENs are reviewed in [Joung, 2012], incorporated herein by reference in its entirety. Similarly, Zinc Finger Nucleases (ZFNs) are another class of modular protein nucleases that can be designed to bind and cut specific DNA sequences [Wright, 2006]. Exemplary ZFNs are reviewed in [Urnov, 2010], incorporated herein by reference in its entirety. Meganucleases can also be used and designed to bind and cut specific DNA sequences. Exemplary meganucleases are reviewed in [Silva, 2011], incorporated herein by reference in its entirety.

In some embodiments, the CRISPR/Cas system may be designed to target RNA molecules. The guide RNA(s) may be designed to target single stranded RNA that is analogous to the guide RNAs designed to target DNA; however, the PAM is provided in trans as a DNA oligonucleotide [O'Connell, 2014]. The DNA oligonucleotide hybridizes to the single stranded RNA target sequence and comprises the non-target strand of the RNA:DNA heteroduplex. The RNA target sequence needs not include the PAM sequence as long as the DNA oligonucleotide provides the PAM sequence to facilitate cleavage. Indeed, the absence of the PAM sequence in the single stranded RNA precludes the CRISPR/Cas system from targeting the encoding DNA sequence.

In various embodiments, TALENs or ZFNs or meganucleases may be substituted for CRISPR/Cas nucleases in an engineered probiotic, provided the TALEN or ZFN or meganuclease is designed to target a specific DNA sequence for degradation. As is generally understood to those skilled in the art, TALENs and ZFNs consist of modular protein domains, each domain conferring binding specificity to a specific DNA base pair. Indivdual modular TALEN domains can target “A,” “T,” “C,” or “G” nucleotides. Thus engineered TALENs comprising a fusion protein of modular TALEN domains can be designed to target an arbitrary and specific base pair sequence [Cermak, 2011]. Likewise, individual modular domains of ZFNs target a variety of 3 base pair sequences. Engineered ZFNs are fusion proteins, typically composed of 3 ZFN modules that target a specific 9 base pair sequence [Maeder, 2008]. Meganucleases target DNA sequences of 10 or more base pairs in length; if this recognition sequence exists in the gene of interest and doesn't exist elsewhere in the genomes of the targeted cellular community, then meganucleases may be substituted for CRISPR/Cas nucleases [Silva, 2011].

### Engineered Horizontal Gene Transfer

Horizontal gene transfer is a major mechanism of transfer of virulence attributes and antibiotic resistance phenotypes within microbial populations. For example, metagenomic analysis of human gut flora indicates that horizontal gene transfer is more prevalent in the human microbiome than in external environments [Smillie, 2011]. The high cell density of the human gastrointestinal tract renders it highly conducive to gene transfer [Ley, 2006]. Mobile genetic elements (MGEs)—including transposons, plasmids, bacteriophage, and pathogenicity islands—are responsible for the acquisition of virulence attributes by otherwise commensal microorganisms [Kaper, 2004]. Horizontal gene transfer is primarily accomplished by one of three mechanisms in bacteria. First, transmission of plasmids via conjugation of a donor bacterium to a recipient bacterium. Second, transformation of a cell with free DNA in the form of a plasmid or nucleic acid fragments. Third, transduction as mediated by a bacteriophage.

In some aspects, the present disclosure provides for a probiotic engineered to include a heterologous, genetic system designed to propagate the gene degradation system within a microbial population. In some embodiments, the heterologous genetic system comprises a synthetic mobile genetic element (MGE) capable of dispersing the gene degradation system to other microbial strains and species in a microbial population. Thus, the engineered probiotic itself need only persist long enough in the microbial population to remove gene(s) of interest from the population and/or to transfer the MGE to other strains within microbial population. Types of known MGEs include conjugative transposons, conjugative plasmids and bacteriophages. Exemplary mobile genetic elements are set forth below in Table 3.

TABLE 3
Mobile genetic elements (MGEs)

| Name | Type | Size | Host range |
|---|---|---|---|
| Tn916 | Conjugative | 18.5 kb, | Gram-positive bacteria with |
| | transposon | 11 genes | lower transmissibility in |
| | | | Gram-negative bacteria |
| RK2 | IncP-1 Conjugative | 60 kb, | Almost all Gram-negative |
| | plasmid | 56 core | bacteria and some |
| | | genes | Gram-positive bacteria |
| | | | including Actinobacteria |
| P1 | Bacteriophage | 90 kb, | Several Gram-negative |
| | | 117 genes | bacteria |
| ZTn5280 | Class I transposon | 8.5 kb | N/A |
| Tn4651 | Class II transposon | 56 kb | N/A |

Conjugative transposons are compact, self-transmissible mobile elements that combine dispersal and translocative functions on a single DNA fragment [Tsuda, 1999; Salyers, 1995]. Conjugative transposons generally reside on the bacterial genome and can self-excise and transfer to recipient cells via conjugation. Exemplary conjugative transposons include Tn916.

Conjugative plasmids offer an alternative embodiment for the MGE of the present disclosure. In some embodiments, the conjugative plasmid is an IncP-1 plasmid since they are known for both their broad-host range and high efficiency self-transmission [Adamczyk, 2003]. Exemplary IncP-1 plasmids maintained in different hosts include E. coli (pRK2013) (α-Proteobacteria), Ralstonia eutropha (pSS50) ((3-Proteobacteria), and RK2 conjugated into Pseudonocardia autotrophica for Gram-positive Actinobacteria. In some embodiments, the MGE is derived from an RK2 compatible conjugative plasmid in which the pir and tra factors have been moved from the plasmid to the chromosome of the engineered probiotic. Host cells that are pir+ and tra+ permit transfer of plasmids bearing RK2 mob elements to new strains. Since the pir and tra factors are provided in trans by the host cell, the RK2 plasmid cannot further propagate in recipient strains lacking these factors. Thus, propagation of the RK2 plasmid is limited only to those strains that make direct contact with the engineered probiotic.

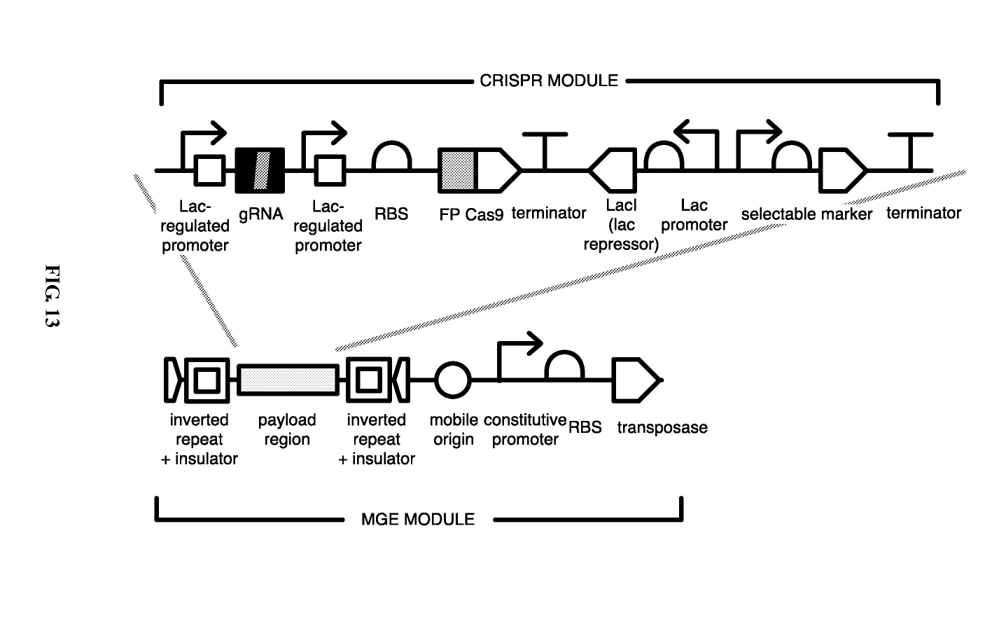

Conjugative plasmids may optionally include a transposon that allows a portion of the plasmid to be stably transferred to the genome of the recipient cell. For example, the tnp transposase from the Tn5 transposon translocates DNA sequences flanked by IS50 repeat sequences [Phadnis, 1986]. Placement of arbitrary DNA between transposon repeats (referred to as a “payload region” in FIG. 13) between transposon repeats (labeled as “inverted repeat”) results in translocation of the payload region to other DNA molecules, including the genome of recipient bacterial cells. A schematic of a typical design including a conjugative plasmid with a transposon is illustrated in FIG. 13. While lac promoter is shown in FIG. 13, one of ordinary skill in the art would understand other inducible promoters can also be used. Exemplary sequences are included as SEQ ID NO:19 and SEQ ID NO:20.

Bacteriophages offer an alternative embodiment for the MGE of the present disclosure. Exemplary bacteriophage includes bacteriophage P1, a temperate phage capable of entering either a lysogenic or lytic state upon infection. Prior published results suggest that P1 has a broad host range among the Gram-negatives including Agrobacterium, Alcaligenes, Citrobacter, Enterobacter, Erwinia, Flavobacterium, Klebsiella, Proteus, Pseudomonas, Salmonella, and Serratia [Murooka, 1979]. Nevertheless, bacteriophages tend to have narrower host ranges than other MGEs like plasmids. Thus, in some embodiments, use of bacteriophage as a transmission vector may necessitate additional engineering of the bacteriophage to broaden its host range. For example, the bacteriophage may be engineered to bypass host restriction-modification systems by eliminating 6 bp palindromic sequences from the MGE and by adding methylase(s) to protect short sites, to expand its replication range by including a broad host range replication origin, and/or to enhance the bacteriophage's ability to penetrate the extracellular matrix by adding display degradative enzymes.

### Organisms or Host Cells for the Engineered Probiotic

The host cell or organism, as disclosed herein, may be chosen from eukaryotic or prokaryotic systems capable of surviving, persisting and/or colonizing in the mammalian gastrointestinal system or the mammalian urinary tract, such as bacterial cells (Gram-negative or Gram-positive), archaea and yeast cells. Suitable organisms can include those bacteria belonging to the Bacteroidetes, Firmicutes, Proteobacteria, Actinobacteria, Verrucomicrobia or Fusobacteria divisions (superkingdoms) of Bacteria. In a preferred embodiment, the host cell/organism is culturable in the laboratory. In some embodiments, host cells/organisms can be selected from Bacteroides species including Bacteroides AFS519, Bacteroides sp. CCUG 39913, Bacteroides sp. Smarlab 3301186, Bacteroides ovatus, Bacteroides salyersiae, Bacteroides sp. MPN isolate group 6, Bacteroides DSM 12148, Bacteroides merdae, Bacteroides distasonis, Bacteroides stercosis, Bacteroides splanchnicus, Bacteroides WH2, Bacteroides uniformis, Bacteroides WH302, Bacteroides fragilis, Bacteroides caccae, Bacteroides thetaiotamicron, Bacteroides vulgatus, and Bacteroides capillosus. In some embodiments, host cells/organisms can be selected from Clostridium species including Clostridium leptum, Clostridium boltaea, Clostridium bartlettii, Clostridium symbiosum, Clostridium sp. DSM 6877(FS41), Clostridium A2-207, Clostridium scindens, Clostridium spiroforme, Clostridium sp. A2-183, Clostridium sp. SL6/1/1, Clostridium sp. GM2/1, Clostridium sp. A2-194, Clostridium sp. A2-166, Clostridium sp. A2-175, Clostridium sp. SR1/1, Clostridium sp. L1-83, Clostridium sp. L2-6, Clostridium sp. A2-231, Clostridium sp. A2-165 and Clostridium sp. SS2/1. In some embodiments, host cells/organisms can be selected from Eubacterium species including Eubacterium plautii, Eubacterium ventriosum, Eubacterium halii, Eubacterium siraeum, Eubacterium eligens, and Eubacterium rectale. In some embodiments, host cells/organisms can be selected from Alistipes finegoldii, Alistipes putredinis, Anaerotruncus colihominis, Allisonella histaminiformans, Bulleida moorei, Peptostreptococcus sp. oral clone CK035, Anaerococcus vaginalis, Ruminococcus bromii, Anaerofustis stercorihominis, Streptococcus mitis, Ruminococcus callidus, Streptococcus parasanguinis, Coprococcus eutactus, Gemella haemolysans, Peptostreptococcus micros, Ruminococcus gnavus, Coprococcus catus, Roseburia intestinalis, Roseburia faecalis, Ruminococcus obeum, Catenibacterium mitsuokai, Ruminococcus torques, Subdoligranulum variabile, Dorea formicigenerans, Dialister sp. E2_20, Dorea longicatena, Faecalibacterium prausnitzii, Akkermansia muciniphila, Fusobacterium sp. oral clone R002, Escherichia coli, Haemophilus parainfluenziae, Bilophila wadsworthii, Desulfovibrio piger, Cornyebacterium durum, Bifidobacterium adolescentis, Actinomyces graevenitzii, Cornyebacterium sundsvallense, Actinomyces odontolyticus, and Collinsella aerofaciens.

In some embodiments, host cells or organisms can be selected from known natural probiotic strains. Exemplary probiotic species include those belonging to the genus Lactobacillus, Bifidobacterium, and/or Streptococcus. Exemplary probiotic strains include Bacillus coagulans GBI-30, 6086, Bifidobacterium animalis subsp. lactis BB-12, Bifidobacterium longum subsp. infantis 35624, Lactobacillus paracasei St11 (or NCC2461), Lactobacillus johnsonii La1 (Lactobacillus johnsonii NCC533), Lactobacillus plantarum 299v, Lactobacillus reuteri ATCC 55730, Lactobacillus reuteri DSM 17938, Lactobacillus reuteri ATCC PTA 5289, Saccharomyces boulardii, Lactobacillus rhamnosus GR-1, Lactobacillus reuteri RC-14, Lactobacillus acidophilus CL1285, Lactobacillus casei LBC80R, Lactobacillus plantarum HEAL 9, Lactobacillus paracasei 8700:2, Streptococcus thermophilus, Lactobacillus paracasei LMG P 22043, Lactobacillus johnsonii BFE 6128, Lactobacillus fermentum ME-3, Lactobacillus plantarum BFE 1685, Bifidobacterium longum BB536 and Lactobacillus rhamnosus LB21 NCIMB 40564.

In some embodiments, the host cell or organism is derived from a laboratory or commensal Escherichia coli strain. Exemplary Escherichia coli strains are set forth below (Table 4). Strain W is the laboratory strain believed to most closely resemble commensal strains [Archer, 2011]. Strain Nissle 1917 has long been used as a probiotic in human under the trade name Mutaflor [Grozdanov, 2004]. The Escherichia coli Collection Of Reference (ECOR) is a collection of commensal Escherichia coli strains that were isolated from the gut of healthy mammals [Ochman 1984]. ECOR strains have not undergone substantial laboratory evolution since their isolation, and are therefore used as model commensal strains.

TABLE 4
Escherichia coli strains

| Strain | Accession |
|---|---|
| E. coli HS | NC_009800 |
| E. coli SE11 | NC_0111415 |
| E. coli SE15 | AP009378 |
| E. coli W | CP002185 |
| E. coli Nissle 1917 | AJ586887-9 |
| E. coli ECOR-08 | ECOR-08 |
| E. coli ECOR-26 | ECOR-26 |
| E. coli ECOR-34 | ECOR-34 |
| E. coli ECOR-36 | ECOR-36 |
| E. coli ECOR-44 | ECOR-44 |
| E. coli ECOR-47 | ECOR-47 |
| E. coli ECOR-51 | ECOR-51 |
| E. coli ECOR-56 | ECOR-56 |
| E. coli ECOR-59 | ECOR-59 |
| E. coli ECOR-61 | ECOR-61 |

In some embodiments, the host cell or organism is derived from the genus Lactobacillus. Exemplary Lactobacillus species include Lactobacillus casei, Lactobacillus lactis, Lactobacillus reuteri, Lactobacillus rhamnosus, Lactobacillus acidophilus, Lactobacillus plantarum, Lactobacillus paracasei, Lactobacillus bulgaricus, Lactobacillus fermentum and Lactobacillus johnsonii.

The host cell or organism, as disclosed herein, may be chosen from eukaryotic or prokaryotic systems capable of surviving, persisting and/or colonizing skin, such as bacterial cells (Gram-negative or Gram-positive), archaea and yeast cells. Suitable organisms can include those bacteria belonging to the Bacteroidetes, Firmicutes, Proteobacteria, and Actinobacteria phyla. In a preferred embodiment, the host cell/organism is culturable in the laboratory. In some embodiments, host cells/organisms can be selected from the genera Staphylococcus, Propionibacterium, Malassezia, Corynebacterium, Brevibacterium, Lactococcus, Lactobacillus, Debaryomyces, and Cryptococcus. Exemplary species include Staphylococcus epidermis, Propionibacterium acnes, Lactococcus lactis, Lactobacillus reuteri and Lactobacillus plantarum.

The host cell or organism, as disclosed herein, may be chosen from eukaryotic or prokaryotic systems capable of surviving, persisting and/or colonizing the environment or substance to be decontaminated, such as bacterial cells (Gram-negative or Gram-positive), archaea and yeast cells.

It should be noted that various engineered strains and/or mutations of the organisms or cell lines discussed herein can also be used.

### Antibiotic-Free Maintenance and Containment of the Engineered Probiotic

In some aspects, the present disclosure provides for a mechanism to select for the maintenance of the engineered probiotic and/or the heterologous genetic system comprising a mobilizable gene targeting and degradation system. Conventionally, plasmid maintenance in host cells or organisms is selected for through the inclusion of antibiotic resistance cassettes and the application of antibiotics to the microbial population. However, the inclusion of antibiotic resistance cassettes in the engineered probiotic of the present disclosure is undesirable since it may lead to unwanted spread of the cassette. Furthermore, use of antibiotics in, for example, the gastrointestinal microbiome, selects against other commensal strains which can promote re-colonization by pathogenic strains particularly in hospital environments [Fekety, 1981]. In a preferred embodiment, the engineered probiotic and/or the heterologous genetic system comprises a nucleic acid encoding one or more genes that confers a selective advantage that is not based on antibiotic resistance.

In some embodiments, the nucleic acid encodes one or more genes that confer the ability to utilize particular carbon source(s) not used by the parent, wildtype host cell or organism from which the engineered probiotic is derived. The inclusion of these carbon source utilization gene(s) confers a selective advantage to any cells carrying the heterologous genetic system when grown in the presence of the corresponding carbon source. Other strains in the microbial population will not be selected against, however, since other carbon sources are available for their growth. In the absence of the corresponding carbon source, the burden of replicating, transcribing and translating the carbon source utilization gene(s) has the additional benefit of limiting the fitness of the engineered probiotic. In this way, the engineered probiotic and/or heterologous genetic system comprising a mobilizable gene targeting and degradation system may be selected for maintenance and dispersal under specific conditions (presence of the carbon source) and selected for containment and loss under other conditions (absence of the carbon source). Co-administration of the carbon source with the probiotic can be used as a means to control the propagation and duration of the probiotic treatment.

In some embodiments, the carbon source utilization gene(s) are derived from the raf operon. The raf operon confers the ability to catabolize the trisaccharide raffinose and has been found on multiple conjugative plasmids [Aslanidis, 1989; Périchon, 2008]. In the raf operon, raffinose inhibits repression of raffinose catabolic genes by the RafR repressor [Ulmke, 1997; Aslandis, 1989]. Raffinose utilization genes include rafA which encodes an alpha-D-galactosidase, rafB which encodes a permease, rafD which encodes an invertase and rafY which encodes a porin. Exemplary raf operon genes are set forth below (Table 5).

TABLE 5
raf operon genes

| Gene | Function | Accession number |
|---|---|---|
| rafR | repressor | M29849 (166 . . . 1176) |
| rafA | alpha-D-galactosidase | M27273.1 (70 . . . 2196) |
| rafB | raffinose permease | M27273.1 (2259 . . . 3536) |
| rafD | raffinose invertase/sucrose hydrolase | M27273.1 (3536 . . . 4966) |
| rafY | porin | U82290 (302 . . . 1696) |

In some embodiments, the carbon source utilization gene(s) are derived from the csc operon. The csc operon confers the ability to catabolize the sugar sucrose [Archer, 2011]. The csc operon comprises cscR which encodes a regulator, cscB which encodes a sucrose transporter, cscA which encodes an invertase, cscK which encodes a fructokinase. Exemplary csc operon genes are set forth below (Table 6).

TABLE 6
csc operon genes

| Gene | Function | Accession number |
|---|---|---|
| cscR | repressor | X81461.2 (7060 . . . 8055) |
| cscB | sucrose transporter | X81461.2 (3171 . . . 4418) |
| cscA | sucrose invertase/sucrose hydrolase | X81461.2 (5619 . . . 7052) |
| cscK | fructokinase | X81461.2 (4480 . . . 5403) |

In some embodiments, the carbon source utilization gene(s) are derived from the xyl operon. The xyl operon confers the ability to catabolize the sugar xylose [Song, 1997]. Exemplary xyl operon genes are set forth below (Table 7).

TABLE 7
xyl operon genes

| Gene | Function | Accession number |
|---|---|---|
| xylR | transcriptional activator | NC_007779.1 (3904258 . . . 3905436) |
| xylA | xylose isomerase | NC_007779.1 (3909650 . . . 3910972) |
| xylB | xyulokinase | NC_007779.1 (3911044 . . . 3912498) |
| xylF | xylose ABC transporter | NC_007779.1 (3908292 . . . 3909284) |
| | subunit | |
| xylG | xylose ABC transporter | NC_007779.1 (3906673 . . . 3908214) |
| | subunit | |
| xylH | xylose ABC transporter | NC_007779.1 (3905514 . . . 3906695) |
| | subunit | |

In some embodiments, the carbon source utilization gene(s) are derived from the ara operon. The ara operon confers the ability to catabolize the sugar arabinose [Miyada, 1984]. Exemplary ara operon genes are set forth below (Table 8).

TABLE 8
ara operon genes

| Gene | Function | Accession number |
|---|---|---|
| araC | transcriptional activator | NC_000913.3 (70387 . . . 71265) |
| araB | L-ribulokinase | NC_000913.3 (68348 . . . 70048) |
| araA | L-arabinose isomerase | NC_000913.3 (66835 . . . 68337) |
| araD | L-ribulose-5-phosphate | NC_000913.3 (65855 . . . 66550) |
| | 4-epimerase | |
| araF | arabinose ABC | NC_000913.3 (1985139 . . . 1986128,) |
| | transporter subunit | |
| araG | arabinose ABC | NC_000913.3 (1983555 . . . 1985069) |
| | transporter subunit | |
| araH | arabinose ABC | NC_000913.3 (1982554 . . . 1983540) |
| | transporter subunit | |

In some aspects, the present disclosure provides for a mechanism to select against the uptake of additional mobile genetic elements by the engineered probiotic of the present disclosure. Various bacterial strains including Escherichia coli, Vibrio chlolerae and Nitrosomonas europaea are known to contain one or more toxin-antitoxin system encoded on their chromosomes; preliminary studies suggest that chromosomally integrated toxin-antitoxin systems may serve to protect the cell from foreign DNA including conjugative plasmids [Saavedra De Bast, 2008]. Thus, in some embodiments, the engineered probiotic of the present disclosure comprises a chromosomally integrated toxin-antitoxin system to restrict uptake and maintenance of foreign DNA from other strains in the microbiome. Exemplary toxin-antitoxin systems, the elements targeted by their cognate toxins, and the cellular process disrupted by the toxins are set forth below (Table 9) [Van Melderen, 2009]. Toxin-antitoxin systems produce a toxin protein that target a cellular process; the antitoxin (typically an RNA or protein) prevents the toxin from disrupting the targeted cellular process. For example, in the MazF system, the MazF toxin protein disrupts RNA translation, and the MazE antitoxin protein binds MazF to ameliorate the toxic activity.

TABLE 9
Toxin-antitoxin systems

| System | Target | Cellular Process |
|---|---|---|
| CcdB | DNA gyrase | replication |
| RelE | translating ribosome | translation |
| MazF | RNAs | translation |
| ParE | DNA gyrase | replication |
| Doc | translating ribosome | translation |
| VapC | RNAs | unknown |
| ζ | unknown | unknown |
| HipA | Ef-Tu | translation |
| HigB | translating ribosome | translation |

In some aspects, the present disclosure provides for a mechanism to select for the functional expression of the CRISPR/Cas based gene targeting and degradation system. It has been demonstrated that natural CRISPR/Cas systems exist that degrade endogenous mRNA transcripts while leaving the corresponding genomic DNA intact [Sampson, 2013]. This is accomplished in Francisella novicida by a scaRNA molecule that forms a complex with tracrRNA and the FTN_1103 mRNA. Since the scaRNA binds specifically to the folded FTN_1103 mRNA, Cas9 selectively degrades the mRNA and not the FTN_1103 DNA sequence. In some embodiments, the mechanism of selection for a functional CRISPR/Cas based gene targeting and degradation system comprises a lethal gene that has been integrated into the chromosome of the engineered probiotic and a CRISPR/Cas system designed to target the mRNA encoded by the lethal gene for degradation while leaving the lethal gene intact. Thus, in some embodiments, the lethal gene is the toxin-encoding gene mazF and the CRISPR/Cas system is designed to target the mazF mRNA toxin for degradation based on its predicted mRNA secondary structure. In an alternative embodiment, the Cas gene is co-located and/or co-transcribed with the mazE gene which encodes the antitoxin. In this embodiment, there is a selection for Cas gene maintenance and/or expression rather than function.

In some embodiments, an engineered probiotic may comprise two or more of the following: one or more targeting and degradation system, one or more mobile genetic elements, one or more antibiotic-free maintenance or containment modules, and one or more functional selection modules. For example, an engineered probiotic may comprise one or more targeting and degradation systems and one or more antibiotic-free maintenance or containment module but no mobile genetic element. Alternatively an engineered probiotic may comprise one or more targeting and degradation systems and one or more functional selection modules but no mobile genetic element.

In some embodiments, an engineered probiotic may comprise a genetic system encoding a nuclease, a MGE, and an antibiotic-free selection module. Genetic systems containing all three modules may serve to transfer from the host cell to cells of interest in a microbial community via the MGE. The nuclease may target a gene of interest for degradation, and the antibiotic-free selection module provides a means of encouraging the propagation of the genetic system in the intended microbial community.

In some embodiments, an engineered probiotic may comprise a genetic system encoding a nuclease and an antibiotic-free selection module. Genetic systems containing these modules may serve to protect a probiotic strain from the acquisition of unwanted genetic elements targeted by the nuclease for degradation.

In some embodiments, an engineered probiotic may comprise a genetic system encoding a MGE and an antibiotic-free selection module. Genetic systems containing these modules may serve to encourage the growth of bacterial species compatible with the host range of the MGE in the intended microbial community. These genetic systems may optionally include additional genetic elements, such as a nuclease, transcriptional activator, or transcriptional repressor.

### Sequences Provided by the Disclosure

Table 10 provides a summary of SEQ ID NOs:1-20 disclosed herein.

TABLE 10
Sequences

| SEQ | |
|---|---|
| ID NO | Sequence |
| 1 | Cas9 nuclease expression cassette |
| 2 | tracrRNA under control of an inducible promoter |
| 3 | CRISPR array targeting cat gene |
| 4 | Plasmid encoding cat gene and fluorescent reporter |
| 5 | Plasmid encoding cat gene and fluorescent reporter |
| 6 | Plasmid encoding cat gene and fluorescent reporter |
| 7 | Plasmid encoding cat gene and fluorescent reporter |
| 8 | Plasmid encoding cat gene and fluorescent reporter |
| 9 | Plasmid encoding tetracycline antibiotic resistance gene |
| 10 | Plasmid encoding guide RNA targeting cat gene, target site #8 |
| 11 | Plasmid encoding guide RNA targeting cat gene, target site #8 |
| 12 | Plasmid encoding guide RNA targeting cat gene, target site #7 |
| 13 | Plasmid encoding guide RNA targeting cat gene, target site #8 |
| 14 | Plasmid encoding guide RNA targeting off-target gene |
| 15 | Plasmid encoding guide RNA targeting cat gene, target site #8 |
| 16 | Plasmid encoding guide RNA targeting cat gene, target site #7 |
| 17 | Mobile conjugative plasmid encoding guide RNA targeting |
| | off-target gene |
| 18 | Mobile conjugative plasmid encoding guide RNA targeting cat |
| | gene, target site #7 |
| 19 | Alternative mobile conjugative plasmid with transposase, |
| | encoding guide RNA targeting off target gene |
| 20 | Alternative mobile conjugative plasmid with transposase, |
| | encoding guide RNA targeting cat gene, target site #7 |

### EXAMPLES

The examples below are provided herein for illustrative purposes and are not intended to be restrictive. For example, while the below examples focus on probiotic engineering, other cells can also be engineered using similar methods and designs.

### Example 1: A Model of Dispersal of the Gene Targeting and Degradation System within a Microbial Population

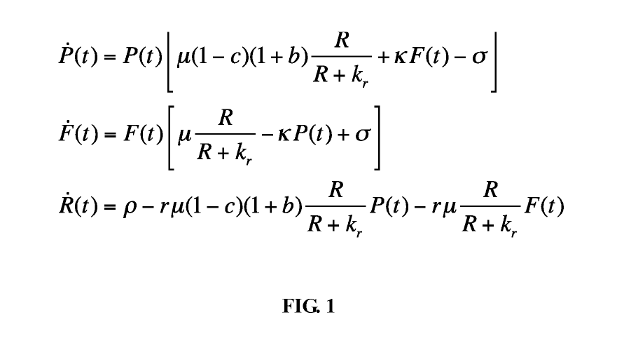

A basic model can be formulated to describe the spread of the gene targeting and degradation system from the engineered probiotic to other members of a microbial community (FIG. 1). In the equations, P(t) and F(t) denotes the subpopulations with and without the gene targeting and degradation system, respectively. R(t) denotes the pool of growth resources available. The model is derived from previously validated models of mobile genetic element dispersion [Bergstrom, 2000; Stewart, 1977]. The other variables are defined as in Table 11.

TABLE 11
Model parameters

| Variable | Definition |
|---|---|
| μ | Growth rate |
| c | Cost of bearing the system |
| b | Benefit of bearing the system |
| κ | Rate of conjugation |
| σ | Rate of plasmid loss due to segregation |
| kᵣ | Critical level of the resource pool |
| ρ | Flow of resources into the environment |
| r | Amount of resources consumed to produce a daughter cell |

The model supports the determination of the initial inoculum density of the engineered probiotic required to achieve colonization, the residence time of the gene targeting and degradation system in the gastrointestinal system, and the ratio of engineered to virulent/antibiotic-resistant microbial cells required for effective clearance of the virulent/antibiotic-resistant genes.

### Example 2: Candidate Target Sites in a Gene of Interest

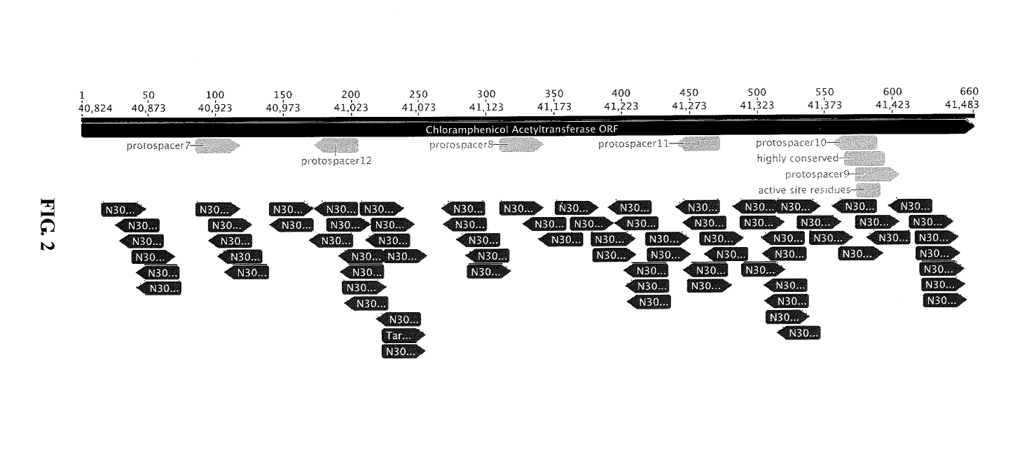

CRISPR/Cas gene targeting and degradation systems may be targeted to select sites within a gene of interest. In the case of systems derived from the Streptomyces pyogenes Cas9 nuclease, target sequences must be immediately 5′ to the sequence NGG, where “N” can be any nucleotide. FIG. 2 depicts a schematic of the Yersinia pestis biovar Orientalis str IP275 chloramphenicol acetyltransferase coding sequence (CAT, Genbank accession NC_009141 40824 . . . 41483). Sites suitable for targeting with the S. pyogenes Cas9 nuclease are annotated in dark gray. Selected target sites and gene features of interest are annotated in light gray. Selected target sites are chosen to coincide with important functional or structural motifs within a gene of interest.

### Example 3: Design and Construction of a CRISPR/Cas Gene Targeting and Degradation System

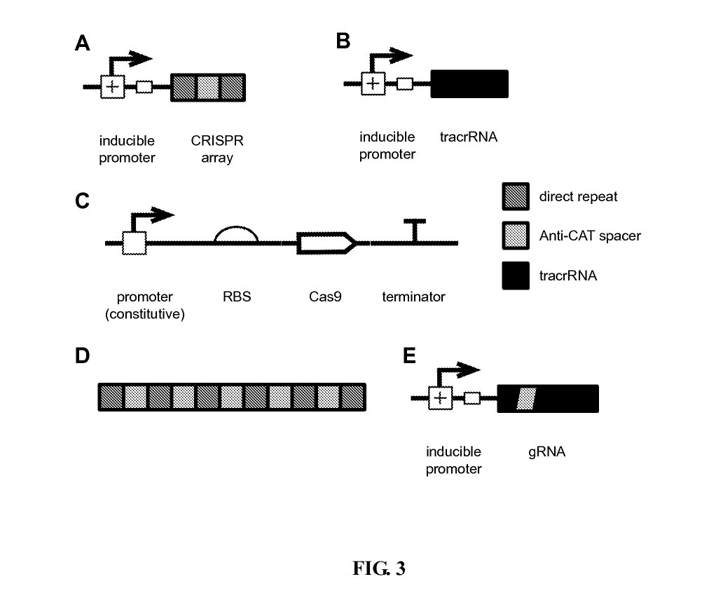

Three components are needed for the proper functioning of a CRISPR/Cas gene targeting and degradation system derived from the Streptomyces pyogenes Cas9 nuclease: the Cas9 protein itself, a CRISPR array containing one or more target DNA “spacers” flanked by CRISPR direct repeats, and a tracrRNA that forms a complex with Cas9 and the crRNA transcribed from the CRISPR array. FIG. 3 depicts example designs of a CRISPR/Cas gene targeting system. The CRISPR array is based on the Streptomyces pyogenes CRISPR array and is designed to target a gene of interest: the chloramphenicol acetyltransferase coding sequence (CAT) described in Example 2. Both the CRISPR array (FIG. 3A) and the tracrRNA (FIG. 3B) are placed under the control of inducible promoters. The Cas9 protein may be expressed constitutively (FIG. 3C). In an alternative design of the CRISPR array, a five spacer array targeted at the CAT gene was constructed (FIG. 3D). However, multiple bands corresponding to different length arrays were observed during gel electrophoresis of arrays synthesized via commercial gene synthesis. The fact that we were unable to obtain a clonal population of this DNA design from commercial gene synthesis providers suggests that this five spacer array design is vulnerable to recombination between repeat units. In an alternative design, the tracrRNA and target spacer are combined into a single guide RNA (FIG. 3E) that is easier to construct and less vulnerable to recombination.

### Example 4: Engineered Probiotic Strains Capable of Repelling Invading Antibiotic Resistance Genes

An engineered probiotic strain was designed and constructed comprising a Streptomyces pyogenes Cas9 nuclease expression cassette (SEQ ID NO:1), a tracrRNA (SEQ ID NO:2) and a CRISPR array (SEQ ID NO:3). The CRISPR array was designed to target a single 30 base pair site in the Yersinia pestis biovar Orientalis str IP275 chloramphenicol acetyltransferase coding sequence (CAT). A second engineered strain was constructed that omitted the CRISPR array to serve as a control strain.

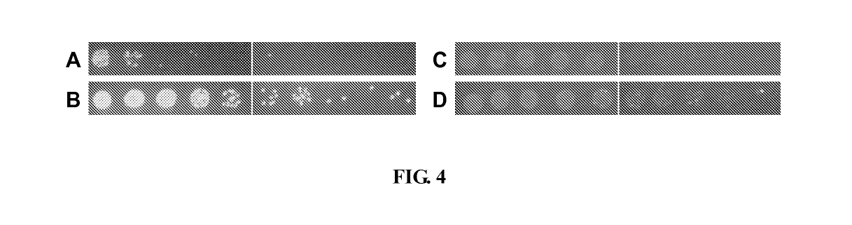

Both strains were challenged via transformation with a plasmid encoding CAT and a fluorescent protein (SEQ ID NO:4). The engineered probiotic strain was found to be effective in repelling plasmids encoding a gene expression cassette comprising CAT (FIG. 4). We observed a 10⁴-10⁵ fold decrease in colony forming units, when selecting on the antibiotic chloramphenicol (FIGS. 4A and 4B show results from the engineered probiotic and control strain, respectively). In the absence of selection, the engineered probiotic is 100% effective in repelling the plasmid encoding the CAT gene (FIGS. 4C and 4D show results from the engineered probiotic and control strain, respectively).

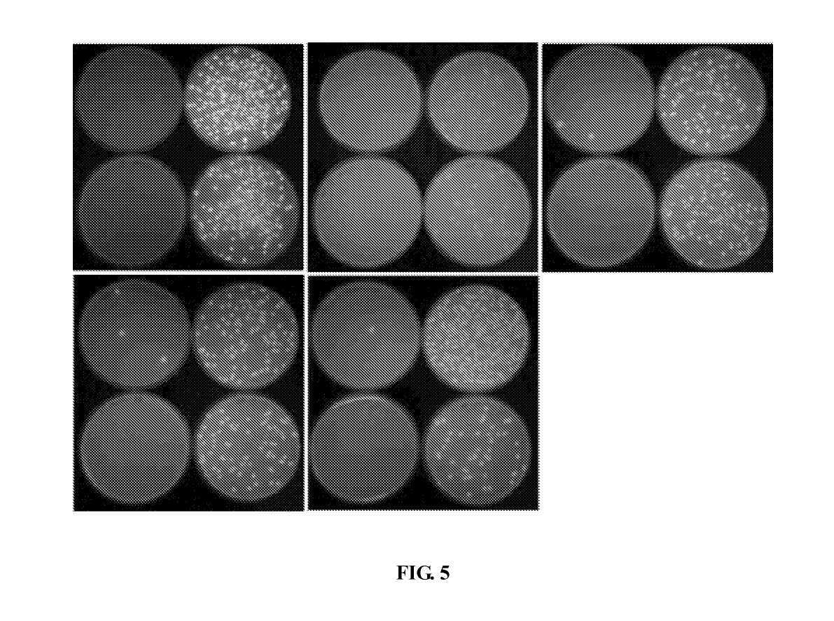

The engineered probiotic and control strain were challenged with five different plasmid designs, each of which encodes the Y. pestis CAT gene (SEQ ID NOs:4-8). In each case, the engineered probiotic successfully repelled the plasmid relative to the control strain even in the presence of chloramphenicol selection (FIG. 5).

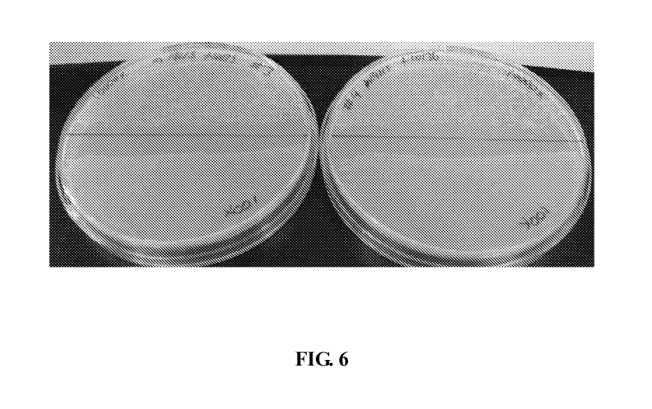

To verify that the observed results were as a result of activity of the CRISPR/Cas gene targeting and degradation system rather than reduced cell competence from maintenance of plasmid encoding the CRISPR array, both the engineered probiotic and control strain were challenged with plasmids that did not encode the Y. pestis CAT gene but did encode a tetracycline antibiotic resistance gene (SEQ ID NO:9). No significant difference in the number of colonies obtained after transformation and growth on tetracycline plates (FIG. 6). These results indicate that the engineered probiotic is specific for the target gene of interest.

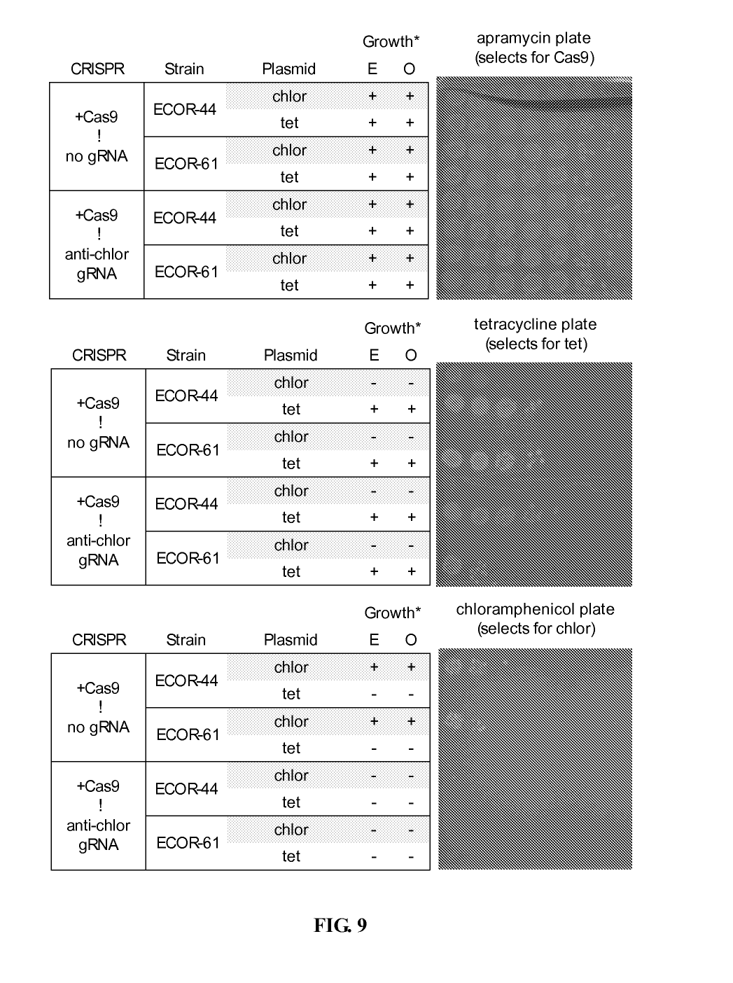

To determine whether this effect was specific to laboratory strains of Escherichia coli, we repeated the experiment using the commensal Escherichia coli strains ECOR-44 and ECOR-61 as hosts for the CRISPR/Cas gene targeting and degradation system. Upon challenging these strains with CAT expressing plasmids, we observed that the commensal strains similarly showed a 10⁴-10⁵ decrease in colony forming units when selecting on the antibiotic chloramphenicol (FIG. 9).

### Example 5: Removal of Previously Acquired Antibiotic Resistance Genes Using a CRISPR/Cas Gene Targeting and Degradation System

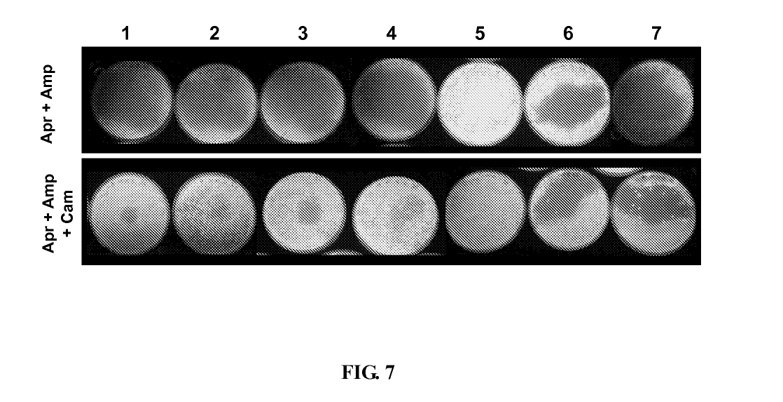

A target strain was designed and constructed comprising a low copy plasmid encoding a Streptomyces pyogenes Cas9 nuclease expression cassette (SEQ ID NO:1) and a high copy plasmid encoding a Yersinia pestis biovar Orientalis str IP275 chloramphenicol acetyltransferase coding sequence (CAT) and fluorescent protein (SEQ ID NO:4). The target strain was subsequently challenged via transformation with guide RNA (gRNA) constructs targeted at different sequences (SEQ ID NOs:10-16). Target strains challenged with gRNAs targeted at the CAT gene (SEQ ID NO:10-13 and SEQ ID NO:15-16) showed a loss of fluorescence and chloramphenicol resistance, whereas a gRNA targeted at a different gene (SEQ ID NO:14) did not impact fluorescence or chloramphenicol resistance phenotypes of the target strain (FIG. 7). Two different on-target gRNAs were tested, each of which targeted a different 20 base pair sequence within the CAT gene; both were effective and did not show evidence of off-target activity irrespective of the identity of the promoter driving transcription of the gRNA or the plasmid propagating the gRNA. The off-target gRNA did not show any evidence of deleterious activity. These results suggest that designed CRISPR/Cas systems can specifically target and remove undesirable DNA elements from target strains.

### Example 6: Design of an Engineered Probiotic

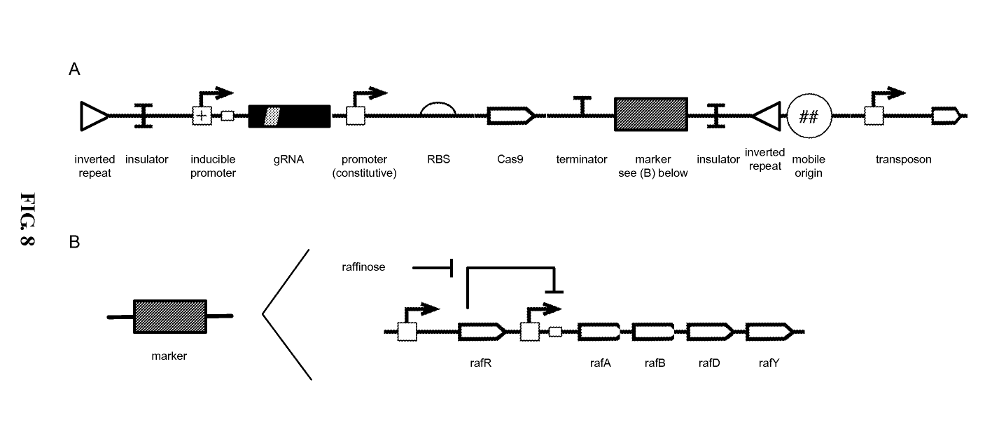

An engineered probiotic was designed comprising a CRISPR/Cas gene targeting and degradation system and selection and containment mechanism derived from the raf operon (FIG. 8). The gene targeting and degradation system and selection and containment mechanism are flanked by inverted repeats and are adjacent to a mobilizable origin and transposon (FIG. 8A) to facilitate dispersal and integration into the genomes of other cells within a microbial community. FIG. 8B shows a selection module design based on the raf operon, in which raffinose inhibits transcription of the rafA, rafB, rafD and rafY genes.

### Example 7: Antibiotic-Free Promotion of Engineered Probiotic Growth

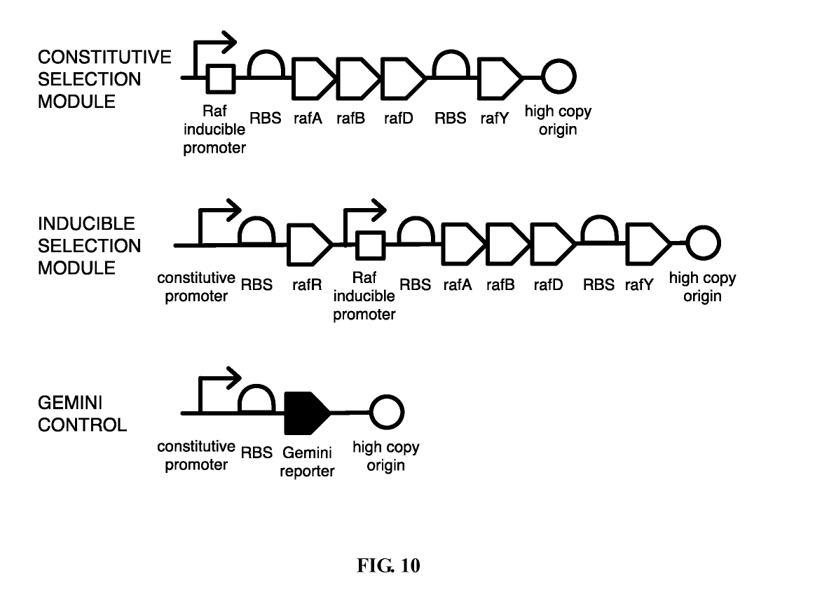

Carbon utilization operons are being used as a means to promote the growth of engineered probiotic strains over competing bacterial strains, without the use of antibiotics. For example, we have demonstrated that the raf operon confers a growth advantage to host Escherichia coli strains when grown in the presence of raffinose. FIG. 10 illustrates how the raf operon can be engineered to function constitutively (“Constitutive Selection Module”) or in an inducible fashion based on the presence of raffinose (“Inducible Selection Module”). The “Gemini Control” design in FIG. 10 is a control plasmid that is identical to the Constitutive Selection Module and Inducible Selection Module plasmids, with the exception that the hybrid fluorescent reporter Gemini is expressed in the place of a raf operon [Martin 2009].

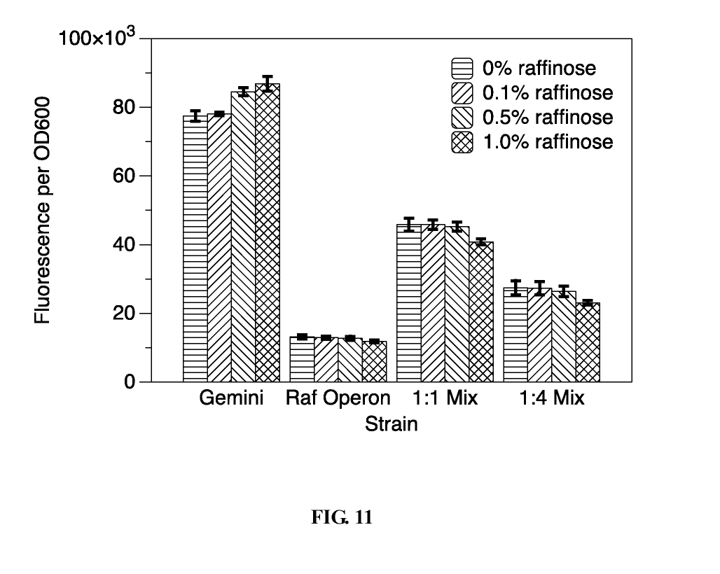

Laboratory strains of Escherichia coli containing the Constitutive Selection Module were placed in competition with strains containing the Gemini Control plasmid. When raffinose is present at a concentration of 1.0% (weight per volume) in the growth media, strains containing the Constitutive Selection Module grow to a higher final titer than identical strains containing the Gemini Control plasmid instead (FIG. 11).

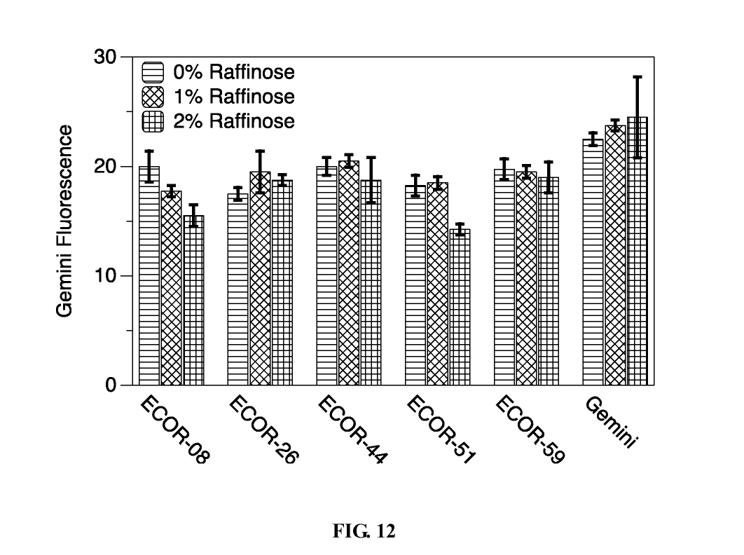

Some commensal strains of Escherichia coli also outgrow Gemini Control strains when transformed with the Constitutive Selection Module. For example, commensal strains E. coli ECOR-08 and E. coli ECOR-51 (each transformed with the Constitutive Selection Module) outgrow a laboratory strain of Escherichia coli transformed with the Gemini Control plasmid (FIG. 12). Laboratory strains of Escherichia coli are better adapted to the growth conditions used in FIG. 12, thus the other ECOR strains tested in this experiment may also exhibit a growth advantage with raffinose when grown in the mammalian gut or similar environments.

### Other Embodiments

The examples have focused on Escherichia coli. Nevertheless, the key concept of using CRISPR/Cas systems to confer the ability to target and degrade undesirable genes of interest is, as one of ordinary skill in the art would understand, extensible to other commensal strains and/or probiotic strains such as other prokaryotes including Lactobacillus or eukaryotic single cell organisms.

The examples have focused on, by way of example only, targeting the chloramphenicol resistance gene for degradation. Nevertheless, the key concept of using an engineered probiotic to target and degrade a gene or genes of interest is, as one of ordinary skill in the art would understand, extensible to other nucleic acids such as genetic elements that encode pathogenic, virulent, virulence factors, alternative antibiotic resistance traits or other undesirable genetic elements. It is also extensible to other nucleic acids such as RNA that is transcribed from a gene of interest, pathogenic element or non-coding genetic elements, as one of ordinary skill in the art would understand.

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### EQUIVALENTS

The present disclosure provides among other things novel methods and systems for synthetic biology. While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

### INCORPORATION BY REFERENCE

The Sequence Listing filed as an ASCII text file via EFS-Web (file name: “134395_010501_Sequence_Listing”; date of creation: Mar. 25, 2015; size: 121,587 bytes) is hereby incorporated by reference in its entirety.

All publications, patents and patent applications referenced in this specification are incorporated herein by reference in their entirety for all purposes to the same extent as if each individual publication, patent or patent application were specifically indicated to be so incorporated by reference.

### REFERENCES CITED

Adamczyk M, Jagura-Burdzy G. Spread and survival of promiscuous IncP-1 plasmids. Acta Biochim Pol. 2003; 50(2):425-53.
Aizenman E, Engelberg-Kulka H, Glaser G. An Escherichia coli chromosomal “addiction module” regulated by guanosine [corrected] 3′,5′-bispyrophosphate: a model for programmed bacterial cell death. Proc Natl Acad Sci USA. 1996 Jun. 11; 93(12):6059-63. Erratum in: Proc Natl Acad Sci USA 1996 Sep. 3; 93(18):9991.
Allen H K. Antibiotic resistance gene discovery in food-producing animals. Curr Opin Microbiol. 2014 June; 19:25-9.
Archer C T, Kim J F, Jeong H, Park J H, Vickers C E, Lee S Y, Nielsen L K. The genome sequence of E. coli W (ATCC 9637): comparative genome analysis and an improved genome-scale reconstruction of E. coli. BMC Genomics. 2011 Jan. 6; 12:9.
Aslanidis C, Schmid K, Schmitt R. Nucleotide sequences and operon structure of plasmid-borne genes mediating uptake and utilization of raffinose in Escherichia coli. J Bacteriol. 1989 December; 171(12):6753-63.
Barrangou R, Fremaux C, Deveau H, Richards M, Boyaval P, Moineau S, Romero D A, Horvath P. CRISPR provides acquired resistance against viruses in prokaryotes. Science. 2007 Mar. 23; 315(5819):1709-12.
Bergstrom C T, Lipsitch M, Levin B R. Natural selection, infectious transfer and the existence conditions for bacterial plasmids. Genetics. 2000 August; 155(4):1505-19.
Bhaya D, Davison M, Barrangou R. CRISPR-Cas systems in bacteria and archaea: versatile small RNAs for adaptive defense and regulation. Annu Rev Genet. 2011; 45:273-97.
Brumbaugh, A. R. & Mobley, H. L. T. Preventing urinary tract infection: progress toward an effective Escherichia coli vaccine. Expert Review of Vaccines. 2012; 11, 663-676.
Cermak T, Doyle E L, Christian M, Wang L, Zhang Y, Schmidt C, Baller J A, Somia N V, Bogdanove A J, Voytas D F. Efficient design and assembly of custom TALEN and other TAL effector-based constructs for DNA targeting. 2011 July; 39(12):e82.
Cong L, Ran F A, Cox D, Lin S, Barretto R, Habib N, Hsu P D, Wu X, Jiang W, Marraffini L A, Zhang F. Multiplex genome engineering using CRISPR/Cas systems. Science. 2013 Feb. 15; 339(6121):819-23.
Deltcheva E, Chylinski K, Sharma C M, Gonzales K, Chao Y, Pirzada Z A et al. ( ). CRISPR RNA maturation by trans-encoded small RNA and host factor RNase III. Nature. 2011; 471 (7340): 602-607.
Deveau H, Barrangou R, Garneau J E, Labonté J, Fremaux C, Boyaval P, Romero D A, Horvath P, Moineau S. Phage response to CRISPR-encoded resistance in Streptococcus thermophilus. J Bacteriol. 2008 February; 190(4):1390-400.
DiCarlo J E, Norville J E, Mali P, Rios X, Aach J, Church G M. Genome engineering in Saccharomyces cerevisiae using CRISPR-Cas systems. Nucleic Acids Res. 2013 April; 41(7):4336-43. doi: 10.1093/nar/gkt135.
Fekety R, Kim K H, Brown D, Batts D H, Cudmore M, Silva J Jr. Epidemiology of antibiotic-associated colitis; isolation of Clostridium difficile from the hospital environment. Am J Med. 1981 April; 70(4):906-8.
Heidelberg J F, Nelson W C, Schoenfeld T, Bhaya D. Germ warfare in a microbial mat community: CRISPRs provide insights into the co-evolution of host and viral genomes. PLoS One. 2009; 4(1):e4169.
Horvath P, Romero D A, Coûté-Monvoisin A C, Richards M, Deveau H, Moineau S, Boyaval P, Fremaux C, Barrangou R. Diversity, activity, and evolution of CRISPR loci in Streptococcus thermophilus. J Bacteriol. 2008 February; 190(4):1401-12.
Horvath P, Coûté-Monvoisin A C, Romero D A, Boyaval P, Fremaux C, Barrangou R. Comparative analysis of CRISPR loci in lactic acid bacteria genomes. Int J Food Microbiol. 2009 Apr. 30; 131(1):62-70.
Ishino Y, Shinagawa H, Makino K, Amemura M, Nakata A. Nucleotide sequence of the iap gene, responsible for alkaline phosphatase isozyme conversion in Escherichia coli, and identification of the gene product. J Bacteriol. 1987 December; 169(12):5429-33.
Jiang W, Bikard D, Cox D, Zhang F, Marraffini L A. RNA-guided editing of bacterial genomes using CRISPR-Cas systems. Nat Biotechnol. 2013 March; 31(3):233-9.
Jinek M, Chylinski K, Fonfara I, Hauer M, Doudna J A, Charpentier E. A programmable dual-RNA-guided DNA endonuclease in adaptive bacterial immunity. Science. 2012 Aug. 17; 337(6096):816-21.
Jinek M, East A, Cheng A, Lin S, Ma E, Doudna J. RNA-programmed genome editing in human cells. Elife. 2013; 2:e00471.
Joung et al., “TALENs: a widely applicable technology for targeted genome editing,” Nat. Rev. Mol. Cell Biol. 14, 49-55 (2012).
Kaper J B, Nataro J P, Mobley H L. Pathogenic Escherichia coli. Nat Rev Microbiol. 2004 February; 2(2):123-40.
Kunin V, Sorek R, Hugenholtz P. Evolutionary conservation of sequence and secondary structures in CRISPR repeats. Genome Biol. 2007; 8(4):R61.
Ley R E, Peterson D A, Gordon J I. Ecological and evolutionary forces shaping microbial diversity in the human intestine. Cell. 2006 Feb. 24; 124(4):837-48.
Li T, Huang S, Zhao X, Wright D A, Carpenter S, Spalding M H, Weeks D P, Yang B. Modularly assembled designer TAL effector nucleases for targeted gene knockout and gene replacement in eukaryotes. 2011 August; 39(14):6315-25.
Maeder, et al., Rapid “open-source” engineering of customized zinc-finger nucleases for highly efficient gene modification. Mol Cell. 2008 Jul. 25; 31(2):294-301.
Mali P, Yang L, Esvelt K M, Aach J, Guell M, DiCarlo J E, Norville J E, Church G M. RNA-guided human genome engineering via Cas9. Science. 2013 Feb. 15; 339(6121):823-6.
Martin L, Che A, Endy D. Gemini, a bifunctional enzymatic and fluorescent reporter of gene expression. PLoS ONE. 2009; 4(11):e7569.
Miyada C G, Stoltzfus L, Wilcox G. Regulation of the araC gene of Escherichia coli: catabolite repression, autoregulation, and effect on araBAD expression. Proc Natl Acad Sci USA. 1984 July; 81(13):4120-4.
Murooka Y, Harada T. Expansion of the host range of coliphage P1 and gene transfer from enteric bacteria to other gram-negative bacteria. Appl Environ Microbiol. 1979 October; 38(4):754-7.
Ochman H, Selander R K. Standard reference strains of Escherichia coli from natural populations. J Bacteriol. 1984 February; 157(2):690-3.
O'Connell M R, Oakes B L, Sternberg S H, East-Seletsky A, Kaplan M, Doudna J A. Programmable RNA recognition and cleavage by CRISPR/Cas9. Nature. 2014 Dec. 11; 516(7530):263-6.
Périchon B, Bogaerts P, Lambert T, Frangeul L, Courvalin P, Galimand M. Sequence of conjugative plasmid pIP1206 mediating resistance to aminoglycosides by 16S rRNA methylation and to hydrophilic fluoroquinolones by efflux. Antimicrob Agents Chemother. 2008 July; 52(7):2581-92.
Phadnis S H, Sasakawa C, Berg D E. Localization of action of the IS50-encoded transposase protein. Genetics. 1986 March 112(3):421-7.
Saavedra De Bast M, Mine N, Van Melderen L. Chromosomal toxin-antitoxin systems may act as antiaddiction modules. J Bacteriol. 2008 July; 190(13):4603-9.
Salyers A A, Shoemaker N B, Stevens A M, Li L Y. Conjugative transposons: an unusual and diverse set of integrated gene transfer elements. Microbiol Rev. 1995 December; 59(4):579-90.
Silbergeld E K, Graham J, Price L B. Industrial Food Animal Production, Antimicrobial Resistance, and Human Health. Annu. Rev. Public. Health. 2008 April; 29(1):151-69.
Silva et al., “Meganucleases and Other Tools for Targeted Genome Engineering: Perspectives and Challenges for Gene Therapy,” Curr Gene Ther. February 2011; 11(1): 11-27.
Smillie C S, Smith M B, Friedman J, Cordero O X, David L A, Alm E J. Ecology drives a global network of gene exchange connecting the human microbiome. Nature. 2011 Oct. 30; 480(7376):241-4.
Song S, Park C. Organization and regulation of the D-xylose operons in Escherichia coli K-12: XylR acts as a transcriptional activator. J Bacteriol. American Society for Microbiology; 1997 November; 179(22):7025-32.
Stewart, F. M. & Levin, B. R., 1977. The Population Biology of Bacterial Plasmids: A PRIORI Conditions for the Existence of Conjugationally Transmitted Factors. Genetics, 87(2), pp. 209-228.
Tsuda M, Tan H M, Nishi A, Furukawa K. Mobile catabolic genes in bacteria. J Biosci Bioeng. 1999; 87(4):401-10.
Ulmke C, Lengeler J W, Schmid K. Identification of a new porin, RafY, encoded by raffinose plasmid pRSD2 of Escherichia coli. J Bacteriol. 1997 September; 179(18):5783-8.
Urnov, et al., “Genome editing with engineered zinc finger nucleases,” Nat. Rev. Genet. 11, 636-646 (2010).
Van Melderen L, De Bast M S. Bacterial Toxin-Antitoxin Systems: More Than Selfish Entities? Rosenberg S M, editor. PLoS Genet. 2009 Mar. 27; 5(3):e1000437.

1. An engineered genetic system, comprising:
a nuclease module designed to specifically target and degrade a nucleic acid of interest encoding a virulence factor, toxin, effector, pathogenic component and/or antibiotic resistance trait; and
a synthetic mobile genetic element (MGE) module capable of dispersing the system from one host cell to another;
wherein the nuclease module comprises a nuclease encoded by a gene located in the MGE module.

2. The system of claim 1, wherein the nuclease module comprises a Cas protein and one or more synthetic crRNAs wherein each crRNA comprises a spacer having a target sequence derived from the nucleic acid of interest.

3. The system of claim 1, wherein the crRNA(s) is transcribed and processed from a CRISPR array.

4. The system of claim 2, wherein the CRISPR array is placed under the control of an inducible promoter or a constitutive promoter.

5. The system of any one of claims 2-4, wherein the nuclease module further comprises a tracrRNA that forms a complex with the Cas protein and crRNA.

6. The system of claim 5, wherein the tracrRNA is placed under the control of an inducible promoter or a constitutive promoter.

7. The system of claim 5 or 6, wherein the tracrRNA and crRNA are provided in a single guide RNA.

8. The system of claim 2, wherein the Cas protein is expressed constitutively or inducibly.

9. The system of claim 2, wherein the target sequence is immediately adjacent to a Protospacer Associated Motif (PAM) in the nucleic acid of interest.

10. The system of claim 9, wherein the Cas protein is Streptomyces pyogenes Cas9 nuclease and the PAM has the NGG sequence 3′ of the target sequence.

11. The system of claim 1, wherein the nuclease comprises a Transcription Activator-Like Effector Nuclease (TALEN) designed to target and degrade the nucleic acid of interest.

12. The system of claim 1, wherein the nuclease comprises a Zinc Finger Nuclease (ZFN) designed to target and degrade the nucleic acid of interest.

13. The system of claim 1, wherein the nuclease comprises a meganuclease designed to target and degrade the nucleic acid of interest.

14. The system of claim 1, wherein the virulence factor, toxin, effector, pathogenic component and/or antibiotic resistance trait are selected from those listed in Tables 1 and 2.

15. The system of claim 1, wherein the MGE module comprises a gene encoding a transposase and a MGE selected from a bacteriophage, conjugative plasmid, or conjugative transposon.

16. The system of claim 15, wherein the transposase is derived from Tn3 or Tn5, and the MGE is derived from Tn916, RK2, P1, Tn5280, or Tn4651.

17. An engineered organism comprising the system of claim 1, for use in the prevention and/or treatment of a disease or infection, the prevention and/or treatment of antibiotic resistance, limiting the spread of antibiotic resistance, and/or decontamination of environmental pathogens.

18. The engineered organism of claim 17, wherein the system is introduced into a host selected from a bacterial cell, archaea cell and/or yeast cell.

19. An engineered probiotic comprising the engineered organism of claim 18, which is an oral probiotic for use in the gastrointestinal tract.

20. An engineered probiotic comprising the engineered organism of claim 18, which is a probiotic for use in the urinary tract.

21. An engineered probiotic comprising the engineered organism of claim 18, which is a topical probiotic for use on the skin.

22. The engineered probiotic of any one of claims 19-21, wherein the host is selected from Bacteroidetes, Firmicutes, Proteobacteria, Actinobacteria, Verrucomicrobia or Fusobacteria divisions of Bacteria.

23. The engineered probiotic of claim 19 or 20, wherein the host is selected from Bacteroides species including Bacteroides AFS519, Bacteroides sp. CCUG 39913, Bacteroides sp. Smarlab 3301186, Bacteroides ovatus, Bacteroides salyersiae, Bacteroides sp. MPN isolate group 6, Bacteroides DSM 12148, Bacteroides merdae, Bacteroides distasonis, Bacteroides stercosis, Bacteroides splanchnicus, Bacteroides WH2, Bacteroides uniformis, Bacteroides WH302, Bacteroides fragilis, Bacteroides caccae, Bacteroides thetaiotamicron, Bacteroides vulgatus, and Bacteroides capillosus.

24. The engineered probiotic of claim 19 or 20, wherein the host is selected from Clostridium species including Clostridium leptum, Clostridium boltaea, Clostridium bartlettii, Clostridium symbiosum, Clostridium sp. DSM 6877(FS41), Clostridium A2-207, Clostridium scindens, Clostridium spiroforme, Clostridium sp. A2-183, Clostridium sp. SL6/1/1, Clostridium sp. GM2/1, Clostridium sp. A2-194, Clostridium sp. A2-166, Clostridium sp. A2-175, Clostridium sp. SR1/1, Clostridium sp. L1-83, Clostridium sp. L2-6, Clostridium sp. A2-231, Clostridium sp. A2-165 and Clostridium sp. SS2/1.

25. The engineered probiotic of claim 19 or 20, wherein the host is selected from Eubacterium species including Eubacterium plautii, Eubacterium ventriosum, Eubacterium halii, Eubacterium siraeum, Eubacterium eligens, and Eubacterium rectale.

26. The engineered probiotic of claim 19 or 20, wherein the host is selected from Alistipes finegoldii, Alistipes putredinis, Anaerotruncus colihominis, Allisonella histaminiformans, Bulleida moorei, Peptostreptococcus sp. oral clone CK035, Anaerococcus vaginalis, Ruminococcus bromii, Anaerofustis stercorihominis, Streptococcus mitis, Ruminococcus callidus, Streptococcus parasanguinis, Coprococcus eutactus, Gemella haemolysans, Peptostreptococcus micros, Ruminococcus gnavus, Coprococcus catus, Roseburia intestinalis, Roseburia faecalis, Ruminococcus obeum, Catenibacterium mitsuokai, Ruminococcus torques, Subdoligranulum variabile, Dorea formicigenerans, Dialister sp. E2_20, Dorea longicatena, Faecalibacterium prausnitzii, Akkermansia muciniphila, Fusobacterium sp. oral clone R002, Escherichia coli, Haemophilus parainfluenziae, Bilophila wadsworthii, Desulfovibrio piger, Cornyebacterium durum, Bifidobacterium adolescentis, Actinomyces graevenitzii, Cornyebacterium sundsvallense, Actinomyces odontolyticus, and Collinsella aerofaciens.

27. The engineered probiotic of claim 19 or 20, wherein the host is selected from the genus Lactobacillus, Bifidobacterium, and/or Streptococcus.

28. The engineered probiotic of claim 19 or 20, wherein the host is selected from Lactobacillus casei, Lactobacillus lactis, Lactobacillus reuteri, Lactobacillus rhamnosus, Lactobacillus acidophilus, Lactobacillus plantarum, Lactobacillus paracasei, Lactobacillus bulgaricus, Lactobacillus fermentum and Lactobacillus johnsonii.

29. The engineered probiotic of claim 19 or 20, wherein the host is selected from Bacillus coagulans GBI-30, 6086, Bifidobacterium animalis subsp. lactis BB-12, Bifidobacterium longum subsp. infantis 35624, Lactobacillus paracasei St11 (or NCC2461), Lactobacillus johnsonii La1 (Lactobacillus johnsonii NCC533), Lactobacillus plantarum 299v, Lactobacillus reuteri ATCC 55730, Lactobacillus reuteri DSM 17938, Lactobacillus reuteri ATCC PTA 5289, Saccharomyces boulardii, Lactobacillus rhamnosus GR-1, Lactobacillus reuteri RC-14, Lactobacillus acidophilus CL1285, Lactobacillus casei LBC80R, Lactobacillus plantarum HEAL 9, Lactobacillus paracasei 8700:2, Streptococcus thermophilus, Lactobacillus paracasei LMG P 22043, Lactobacillus johnsonii BFE 6128, Lactobacillus fermentum ME-3, Lactobacillus plantarum BFE 1685, Bifidobacterium longum BB536 and Lactobacillus rhamnosus LB21 NCIMB 40564.

30. The engineered probiotic of claim 19 or 20, wherein the host is selected from an Escherichia coli strain.

31. The engineered probiotic of claim 19, 20 or 30, wherein the host is selected from E. coli HS, E. coli SE11, E. coli SE15, E. coli W, and E. coli Nissle 1917.

32. The engineered probiotic of claim 21, wherein the host is selected from the genera Staphylococcus, Propionibacterium, Malassezia, Corynebacterium, Brevibacterium, Lactococcus, Lactobacillus, Micrococcus, Debaryomyces, and Cryptococcus.

33. The engineered probiotic of claim 21, wherein the host is selected from Staphylococcus epidermis, Staphylococcus saprophyticus, Propionibacterium acnes, Propionibacterium avidum, Lactococcus lactis, Lactobacillus reuteri and Lactobacillus plantarum.

34. A method for prevention and/or treatment of a disease or infection, for prevention and/or treatment of antibiotic resistance, and/or for limiting the spread of antibiotic resistance, comprising administering an effective amount of the engineered probiotic of any one of claims 19-33 to a subject in need thereof.

35. A population of cells, comprising at least one engineered organism of claim 17, wherein the MGE module in the at least one engineered organism is capable of spreading the engineered genetic system into other cells in the population.


SEQUENCE LISTING (20 sequences)

SEQ ID NO: 1 - DNA, 4409 bp, Unknown
  Synthetic
mgcccacagctaacaccacgtcgtccctatctgctgccctaggtctatgagtggttgctggataactttacgggcatgcataaggctcgtatgatatattcagggagaccacaacggtttccctctacaaataattttgtttaacttttcacacaggaaagtactagatggacaagaaatactcgatagggctggatatcgggaccaactccgtcggttgggctgttatcacggatgaatataaggtgcccagcaaaaagttcaaggtgctaggtaacaccgaccggcacagtatcaaaaaaaacttgataggagcgttgctgtttgacagtggtgagaccgctgaagctacgcgccttaaaaggaccgcgcgccgtagatatacccgcaggaagaaccgcatttgctatctccaagagattttttcgaacgagatggctaaagtagatgatagtttctttcatcgattggaagaaagctttttagttgaagaagacaagaagcatgaacgccatccaatttttggtaacatagtagatgaagtggcgtatcatgagaaatatccgacgatctatcatctacgaaaaaaattagtagatagcacggataaagctgatttacggctgatctatttagcactggctcatatgattaagtttcgcggccattttttaattgagggtgatctgaacccagataacagtgatgttgacaaactctttatccaattagtacagacttacaaccagctgtttgaagaaaatccaattaatgccagtggtgtagatgcgaaagctattttgagcgcccgattaagtaaatcgcgtcgactggaaaaccttattgcgcaacttcctggcgagaagaaaaacgggctgtttggaaaccttattgcgttatcgttaggcttaactccaaactttaaatcgaactttgatttagccgaagatgcgaaactgcaattgtcgaaagatacgtacgatgatgatctggataacctgttagctcagattggtgatcagtatgcggatttatttttagccgcgaagaacctgtcggatgcgattctgttgtcggatatcctccgtgtaaacacggaaataacgaaggcgcctctctcggcgtcgatgattaaacggtacgatgaacatcatcaggacttaacgttgctgaaagcgctggtgcgacagcagttgccggaaaagtataaagaaatcttttttgatcagtcgaaaaatggttatgccggctatattgatggaggtgcgtcccaggaagaattttataaatttatcaaaccgattctggaaaaaatggatggcacggaggaactgttggttaaactcaaccgcgaagatttgctacggaagcagaggacttttgacaatgggagcattcctcatcagattcacttgggcgagctacatgcgattttgcgtcgtcaggaagacttttatccgtttctgaaagacaaccgcgagaagattgaaaaaatcttgacgtttcgaatcccatattatgtgggcccgttagctcgcgggaacagtcgctttgcctggatgacgaggaagtcggaagaaaccattactccgtggaactttgaagaagtggtcgataaaggcgcgtcggcgcagtcgtttattgaacggatgaccaattttgataaaaacttgccgaacgaaaaagtactcccgaaacatagtttattgtatgagtattttacagtgtataatgaattaaccaaggtcaaatatgtgacggaaggtatgcgaaaaccggcctttttgtcgggcgaacaaaagaaagcaattgtggatctgcttttcaaaaccaaccgaaaagtaactgtgaagcagctgaaagaagattatttcaaaaaaatagaatgctttgatagtgtggaaatttcgggtgtggaagatcgttttaacgcgtcgctgggcacttaccatgatttactcaaaattattaaagataaagattttttagataacgaagaaaacgaagatatcctggaggatattgtgctgaccttaactctgtttgaagatagagagatgattgaggaacgtttgaaaacctatgcgcacctttttgatgataaggttatgaaacaattgaaacgccggcgctatacgggctggggtcgcttaagccgaaaattaattaacggcattagagataagcagagcgggaaaaccatactggattttttaaaatcggatggctttgcaaaccggaactttatgcaactaatccatgatgatagtttaacctttaaagaagacattcagaaagcccaggttagcggtcagggggatagtctgcatgaacatattgccaacctggcgggctccccagcgattaaaaaaggcattctgcaaacggtaaaagtggtggatgaattagtcaaagtaatgggaaggcataagccggaaaacatcgtgattgaaatggcccgcgaaaaccaaaccacgcagaaggggcaaaaaaactcacgagagcgcatgaaacgaatcgaagaaggcatcaaagaactgggtagtcaaattttgaaagagcatccagtggaaaacacgcagttacagaacgaaaagctttatctttattatcttcagaacggtcgtgacatgtatgttgaccaggaactggatattaaccgcctgagtgattatgatgtcgatcacattgtgccgcagagtttcttgaaagacgattcgatagacaacaaggtcctgacacgcagcgataaaaaccgcggcaaatcagataatgtgccgagtgaagaagtagtcaaaaagatgaaaaattattggcgtcagttgctcaatgcaaagctgatcacgcagcgcaagtttgataacctgacaaaagcggaacgcggtggcttaagtgaattggataaagcgggctttatcaaacggcagttagtggaaacgcggcagatcacgaagcatgttgcccagattttagatagtcggatgaacacgaaatacgatgaaaacgataaattgattcgagaggtgaaagttattactctgaaaagcaaactggtgagcgacttccgaaaagatttccagttctataaagtacgcgagattaataactaccatcatgcacatgatgcttatctcaacgcagtcgtgggtacggcgttaattaagaaatatccgaaattggaatcagagtttgtctatggcgattataaagtgtatgatgtgcgcaaaatgattgcgaagtcggagcaggaaatagggaaagccactgccaaatatttcttttacagcaacatcatgaatttcttcaaaaccgaaattaccttggccaacggtgagattcggaaacggccactcatcgaaacgaacggagaaacgggtgaaattgtctgggataaaggacgagattttgcaaccgttcggaaagtattgtccatgcctcaggtcaacattgtcaagaaaaccgaagtacaaaccgggggtttctccaaggagtcgattctgccgaaacgcaactcagacaagttgattgcgcgcaaaaaagactgggatccgaaaaaatatggcggcttcgattccccgacagtagcgtactcggtcctcgtcgtggcgaaggtcgaaaaagggaaatccaagaagctgaaatccgtgaaagagctgctcgggatcaccatcatggagcgctcctccttcgagaagaaccccatcgacttcctggaggcgaagggctacaaggaggtgaagaaggacctgatcatcaagctccccaagtactccctctttgagctggagaacggccgcaagaggatgctcgcaagtgcaggtgaattacaaaaaggtaatgaactagcgctaccgtccaaatatgttaactttctgtatctggcgagtcattatgaaaagttaaagggcagtccggaagataatgaacagaaacagttatttgttgagcaacataagcattatctggatgagattattgagcagatcagtgaatttagcaagcgcgtgattctggccgatgcaaacctggataaagtgttgagtgcctataataaacatcgtgacaaaccgatacgcgaacaggccgaaaacattattcatctgtttacattaacaaacttgggtgcgcctgcggcgtttaaatattttgataccaccattgatcgcaaacgatatacaagcaccaaagaagtgctggatgcaacgttgatccatcagtctatcacgggcttgtatgaaacccggattgatttaagtcaactcggtggcgactaagccccaggcatcaaataaaacgaaaggctcagtcgaaagactgggcctttcgttttatctgttgtttgtcggtgaacgctctctactagagtcacactggctcaccttcgggtgggcctttctgcgtttatagcc

SEQ ID NO: 2 - DNA, 259 bp, Unknown
  Synthetic
mgcctccctatcagtgatagagattgacatccctatcagtgatagagatactgagcacgccggaaccattcaaaacagcatagcaagttaaaataaggctagtccgttatcaacttgaaaaagtggcaccgagtcggtgcgccccggcttatcggtcagtttcacctgatttacgtaaaaacccgcttcggcgggtttttgcttttggaggggcagaaagatgaatgactgtccacgacgctatacccaaaagaaagcc

SEQ ID NO: 3 - DNA, 376 bp, unknown
  Synthetic
mgcctccctatcagtgatagagattgacatccctatcagtgatagagatactgagcacgccaggggcttttcaagactgaagtctagctgagacaaatagtgcgattacgaaattttttagacaaaaatagtctacgaggttttagagctatgctgttttgaatggtcccaaaacatggcaatgaaagacggtgagctggtgatagttttagagctatgctgttttgaatggtctccattcgccccaggcatcaaataaaacgaaaggctcagtcgaaagactgggcctttcgttttatctgttgtttgtcggtgaacgctctctactagagtcacactggctcaccttcgggtgggcctttctgcgtttatagcc

SEQ ID NO: 4 - DNA, 3223 bp, Unknown
  Synthetic
maaaaaaaatccttagctttcgctaaggatgatttctggaattcgcggccgcttctagagcccacagctaacaccacgtcgtccctatctgctgccctaggtctatgagtggttgctggataactttacgggcatgcataaggctcgtatgatatattcagggagaccacaacggtttccctctacaaataattttgtttaacttttcacacaggaaagtactagatgaccatgattacggattcactggccgtcgttttacaacgtcgtgactgggaaaaccctggcgttacccaacttaatcgccttgcagcacatccccctttcgcaagctggcgtaatagcgaggaggcccgcaccgatcgcccttcccaacagttgcgcagcctgaatggcgaatggcgcgcgggcggcagcgaaggcggcggcagcgaacatcatcatcatcatcatggcagcgaacgtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactaccctgacctatggtgttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaagtaagccacacgcgctctcccccctccggtgtaatcgggggagagcgcgtgtccgctgcagtccggcaaaaaagggcaaggtgtcaccaccctgccctttttctttaaaaccgaaaagattacttcgcgttatgcaggcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccacaggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagctcgaggcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgcccgatcaactcgagtgccacttgacgtctaagaaaccattattatcatgacattaacctataaaaataggcgtatcacgaggcagaatttcagat

SEQ ID NO: 5 - DNA, 5391 bp, Unknown
  Synthetic
mttatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccataaaagaggagaaatactagatgaccatgattacggattcactggccgtcgttttacaacgtcgtgactgggaaaaccctggcgttacccaacttaatcgccttgcagcacatccccctttcgcaagctggcgtaatagcgaggaggcccgcaccgatcgcccttcccaacagttgcgcagcctgaatggcgaatggcgcgcgggcggcagcgaaggcggcggcagcgaacatcatcatcatcatcatggcagcgaacgtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactaccctgacctatggtgttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaagtaatactagtagcggccgctgcaggagtcactaagggttagttagttagattagcagaaagtcaaaagcctccgaccggaggcttttgactaaaacttcccttggggttatcattggggctcactcaaaggcggtaatcagataaaaaaaatccttagctttcgctaaggatgatttctgctagagctgtcagaccaagtttacgagctcgcttggactcctgttgatagatccagtaatgacctcagaactccatctggatttgttcagaacgctcggttgccgccgggcgttttttattggtgagaatccaagcactagggacagtaagacgggtaagcctgttgatgataccgctgccttactgggtgcattagccagtctgaatgacctgtcacgggataatccgaagtggtcagactggaaaatcagagggcaggaactgctgaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggacatgtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacataacggactaagaaaaacactacacgatgctttaactgcaaaaattcagctcaccagttttgaggcaaaatttttgagtgacatgcaaagtaagtatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttacatatcaaagggaaaactgtccatatgcacagatgaaaacggtgtaaaaaagatagatacatcagagcttttacgagtttttggtgcattcaaagctgttcaccatgaacagatcgacaatgtaactactagaggttgatcgggcacgtaagaggttccaactttcaccataatgaaataagatcactaccgggcgtattttttgagttatcgagattttcaggagctaaggaagctaaaatggagaaaaaaatcacgggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaacgctcacccggagtttcgtatggccatgaaagacggtgagctggtgatctgggatagtgttcacccttgttacaccgttttccatgagcaaactgaaacgttttcgtccctctggagtgaataccacgacgatttccggcagtttctccacatatattcgcaagatgtggcgtgttacggtgaaaacctggcctatttccctaaagggtttattgagaatatgttttttgtctcagccaatccctgggtgagtttcaccagttttgatttaaacgtggccaatatggacaacttcttcgcccccgttttcacgatgggcaaatattatacgcaaggcgacaaggtgctgatgccgctggcgatccaggttcatcatgccgtttgtgatggcttccatgtcggccgcatgcttaatgaattacaacagtactgtgatgagtggcagggcggggcgtaataatactagctccggcaaaaaaacgggcaaggtgtcaccaccctgccctttttctttaaaaccgaaaagattacttcgcgtttgccacctgacgtctaagaaaaggaatattcagcaatttgcccgtgccgaagaaaggcccacccgtgaaggtgagccagtgagttgattgctacgtaattagttagttagcccttagtgactcgaattcgcggccgcttctagagacctcgtgt

SEQ ID NO: 6 - DNA, 3848 bp, Unknown
  Synthetic
mcacagctaacaccacgtcgtccctatctgctgccctaggtctatgagtggttgctggataactttacgggcatgcataaggctcgtatgatatattcagggagaccacaacggtttccctctacaaataattttgtttaacttttcacacaggaaagtactagatggtaagcaagggcgaggagctgttcaccggggtggtgcccatcctggtcgagctggacggtgacgtaaacggtcacaagttcagcgtgagtggcgagggcgagggtgatgccacctacggtaagctgaccctgaagctgatctgcaccaccggtaagctgcccgtgccctggcccacccttgtgaccaccctgggctacggtctgcaatgcttcgcccgttaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaaggccaatgtcgagtaacatggtaagcaagggcgaggagaataacatggccatcatcaaggagttcatgcgcttcaaggtgcacatggagggcagtgtgaacggccacgagttcgagatcgagggcgagggcgagggccgtccctacgaggcctttcagaccgctaagctgaaggtgaccaagggtggccccctgcccttcgcctgggacatcctgtcccctcagttcatgtacggctccaaggtctacattaagcacccagccgacatccccgactacttcaagctgtccttccccgagggcttcaggtgggagcgcgtgatgaacttcgaggacggcggcattattcacgttaaccaggacagttccctgcaagacggcgtgttcatctacaaggtgaagctgcgcggcaccaacttccccagtgacggccccgtaatgcagaagaagaccatgggctgggaggccagtgaggagcggatgtaccccgaggacggcgccctgaagtctgagatcaaaaagaggctgaagctgaaggacggcggccactacgccgccgaggtcaagaccacctacaaggccaagaagcccgtgcagctgcccggcgcctacatcgtcgacatcaagttggacatcgtgtcccacaacgaggactacaccatcgtggaacagtacgaacgcgccgagggccgccactccaccggcggcatggacgagctgtacaaggcctaaccaggcatcaaataaaacgaaaggctcagtcgaaagactgggcctttcgttttatctgttgtttgtcggtgaacgctctctactagagtcacactggctcaccttcgggtgggcctttctgcgtttatagccacacgcgctctcccccctccggtgtaatcgggggagagcgcgtgtccgctgcagtccggcaaaaaagggcaaggtgtcaccaccctgccctttttctttaaaaccgaaaagattacttcgcgttatgcaggcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccacaggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagctcgaggcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgcccgatcaactcgagtgccacttgacgtctaagaaaccattattatcatgacattaacctataaaaataggcgtatcacgaggcagaatttcagataaaaaaaatccttagctttcgctaaggatgatttctggaattcgcggccgcttctagagcc

SEQ ID NO: 7 - DNA, 3210 bp, Unknown
  Synthetic
mcacagctaacaccacgtcgtccctatctgctgccctaggtctatgagtggttgctggataactttacgggcatgcataaggctcgtatgatatattcagggagaccacaacggtttccctctacaaataattttgtttaactttgccagtacgtctgagcgtgatacccgctcactgaagatggcccggtagggccgaaacgtacctctacaaataattttgtttaagcctcacacaggaaagtactagatggtaagcaagggcgaggagctgttcaccggggtggtgcccatcctggtcgagctggacggtgacgtaaacggtcacaagttcagcgtgagtggcgagggcgagggtgatgccacctacggtaagctgaccctgaagctgatctgcaccaccggtaagctgcccgtgccctggcccacccttgtgaccaccctgggctacggtctgcaatgcttcgcccgttaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaaggcctaaccaggcatcaaataaaacgaaaggctcagtcgaaagactgggcctttcgttttatctgttgtttgtcggtgaacgctctctactagagtcacactggctcaccttcgggtgggcctttctgcgtttatagccacacgcgctctcccccctccggtgtaatcgggggagagcgcgtgtccgctgcagtccggcaaaaaagggcaaggtgtcaccaccctgccctttttctttaaaaccgaaaagattacttcgcgttatgcaggcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccacaggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagctcgaggcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgcccgatcaactcgagtgccacttgacgtctaagaaaccattattatcatgacattaacctataaaaataggcgtatcacgaggcagaatttcagataaaaaaaatccttagctttcgctaaggatgatttctggaattcgcggccgcttctagagcc

SEQ ID NO: 8 - DNA, 3204 bp, Unknown
  Synthetic
macacgcgctctcccccgtcacgtacgtcgcagtacgtgctcttcgtcacactggctcaccttcgggtgggcctttctgcgtttatagcccacagctaacaccacgtcgtccctatctgctgccctaggtctatgagtggttgctggataactttacgggcatgcataaggctcgtaatatatattcagggagaccacaacggtttccctctacaaataattttgtttaacttttcacacaggaaagtactagatggtaagcaagggcgaggagaataacatggccatcatcaaggagttcatgcgcttcaaggtgcacatggagggcagtgtgaacggccacgagttcgagatcgagggcgagggcgagggccgtccctacgaggcctttcagaccgctaagctgaaggtgaccaagggtggccccctgcccttcgcctgggacatcctgtcccctcagttcatgtacggctccaaggtctacattaagcacccagccgacatccccgactacttcaagctgtccttccccgagggcttcaggtgggagcgcgtgatgaacttcgaggacggcggcattattcacgttaaccaggacagttccctgcaagacggcgtgttcatctacaaggtgaagctgcgcggcaccaacttccccagtgacggccccgtaatgcagaagaagaccatgggctgggaggccagtgaggagcggatgtaccccgaggacggcgccctgaagtctgagatcaaaaagaggctgaagctgaaggacggcggccactacgccgccgaggtcaagaccacctacaaggccaagaagcccgtgcagctgcccggcgcctacatcgtcgacatcaagttggacatcgtgtcccacaacgaggactacaccatcgtggaacagtacgaacgcgccgagggccgccactccaccggcggcatggacgagctgtacaaggcctaaccaggcatcaaataaaacgaaaggctcagtcgaaagactgggcctttcgttttatctgttgtttgtcggtgaacgctctctactagagtcacactggctcaccttcgggtgggcctttctgcgtttatagcccgaagagcacgtactgctaagctgactgcgggggagagcgcgtgtccgctgcagtccggcaaaaaagggcaaggtgtcaccaccctgccctttttctttaaaaccgaaaagattacttcgcgttatgcaggcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccacaggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagctcgaggcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgcccgatcaactcgagtgccacttgacgtctaagaaaccattattatcatgacattaacctataaaaataggcgtatcacgaggcagaatttcagataaaaaaaatccttagctttcgctaaggatgatttctggaattcgcggccgcttctagagcc

SEQ ID NO: 9 - DNA, 2684 bp, Unknown
  Synthetic
mcacagctaacaccacgtcgtccctatctgctgccctaggtctatgagtggttgctggataactttacgggcatgcataaggctcgtaatatatattcagggagaccacaacggtttccctctacaaataattttgtttaacttttcacacaggaaagtactagatgtcgagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcgtaagcctcacacaggaaagtactagatgaaatctaacaatgcgctcatcgtcattctcggcaccgtcaccctggacgctgtaggcataggcttggttatgccggtactgccgggcctcttgcgggatatcgtccattccgacagtattgccagtcactatggcgtgctgcttgcgctctatgcgttgatgcaatttctttgcgcacccgttctcggagccctgtccgaccgctttggccgccgtccagtcctgctcgcttcgctccttggagccactatcgactacgcgatcatggcgaccacacccgtcctgtggattctctacgccggacgcatcgtggcgggcatcacgggtgccacaggtgcggttgctggtgcctatatcgccgacatcaccgatggggaagatcgggctcgccacttcgggctcatgagcgcttgtttcggcgtgggtatggtggcaggccccgtggccgggggactgttgggtgccatctccttgcatgcaccattccttgcggcggcggtgctcaacggcctcaacctcctcctgggctgcttccttatgcaggaatcgcataagggagagcgccgtccgatgcccttgcgtgccttcaatccagtcagctccttccggtgggcgcggggcatgactatcgtcgccgcacttatgactgttttctttatcatgcaactcgtaggacaggttccggcagcgctctgggtcattttcggcgaggaccgctttcgctggagcgcgacgatgatcggcctgtcgcttgcggtattcggaatcttgcacgccctcgctcaagccttcgtcacgggccccgccaccaaacgtttcggcgagaagcaggccattatcgcgggcatggcggccgacgcgctgggctacgtcttgctggcgttcgcgacgcgcggctggatggccttccccattatgattcttctcgcttccggcggcatcggtatgcccgcgttgcaggccatgctgtcccgccaagtagatgacgaccatcagggacagcttcaagggtcgctcgcggctcttaccagcctcacttcgatcattggaccgctgatcgtcacggcgatttatgccgcctcggcgagcacatggaacgggttggcatggattgtaggtgccgccctttaccttgtctgcctccccgcgttgcgtcgcggtgcatggagccgggccacctcgacctaataagcccccgtagaaaagatcaaaagatcttcttgagatcctttttttctgcgcgtaatctgctacttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctgtggaaaaacgccagcaacgcggcctttttacggttcctggccttttgctggccttttgctcacatgcc

SEQ ID NO: 10 - DNA, 2173 bp, Unknown
  Synthetic
maattgtgagcggataacaattacgagcttcatgcacagtgaaatcatgaaaaatttatttgctttgtgagcggataacaattataatatgtggaattgtgagcgctcacaattccacaacggtttccctctagaaataattttgtttaacttttgccgtgaataccacgacgatttcgttttagagctagaaatagcaagttaaaataaggctagtccgttatcaacttgaaaaagtggcaccgagtcggtggtgcgccccaggcatcaaataaaacgaaaggctcagtcgaaagactgggcctttcgttttatctgttgtttgtcggtgaacgctctctactagagtcacactggctcaccttcgggtgggcctttctgcgtttatagcccacagctaacaccacgtcgtccctatctgctgccctaggtctatgagtggttgctggataactttacgggcatgcataaggctcgtaatatatattcagggagaccacaacggtttccctctacaaataattttgtttaacttttcacacaggaaacctactagatgagtattcaacatttccgtgtcgcccttattcccttttttgcggcattttgccttcctgtttttgctcacccagaaacgctggtgaaagtaaaagatgccgaagatcagttgggtgcacgtgtgggttacatcgaactggacctcaacagcggtaagattcttgagagttttcgccccgaagaacgtttcccaatgatgagcacttttaaagttctgctctgtggcgcggtattatcccgtattgacgccgggcaagagcaactcggtcgccgcatacactattctcagaatgacttggttgagtactcaccagtcacagaaaagcatcttacggacggcatgacagtacgcgaattatgcagcgctgccataaccatgagtgataacacggcggccaacttacttctgacaacgatcggaggaccgaaggagcttaccgcttttttgcacaacatgggtgatcatgtaactcgccttgatcgttgggaaccggagctgaatgaagccataccaaacgacgagcgtgacaccacgatgcctgtagctatggcaacaacgttgcgcaaactcttaactggcgaacttcttactctcgcttcccggcaacaattaatagactggatggaggcggataaagttgcaggaccacttctgcgctcggcccttccggctggctggtttattgctgataaatctggagccggtgagcgtgggtcccgcggtattattgcagccctggggccagatggtaagccctcccgtatcgtagttatctacacgacggggagccaggcaactatggacgaacgtaatcgccagatcgctgagataggtgcctccctgattaagcattggtaagcccttccgcttcctcgctcactgactcgctacgctcggtcgttcgactgcggcgagcggtgtcagctcactcaaaagcggtaatacggttatccacagaatcaggggataaagccggaaagaacatgtgagcaaaaagcaaagcaccggaagaagccaacgccgcaggcgtttttccataggctccgcccccctgacgagcatcacaaaaatcgacgctcaagccagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgttggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccattggtaactgatttagaggactttgtcttgaagttatgcacctgttaaggctaaactgaaagaacagattttggtgagtgcggtcctccaacccacttaccttggttcaaagagttggtagctcagcgaaccttgagaaaaccaccgttggtagcggtggtttttctttatttatgagatgatgaatcaatcggtctatcaagtcaacgaacagctattccgttgcc

SEQ ID NO: 11 - DNA, 2073 bp, Unknown
  Synthetic
mtccctatcagtgatagagattgacatccctatcagtgatagagatactgagcacgccgtgaataccacgacgatttcgttttagagctagaaatagcaagttaaaataaggctagtccgttatcaacttgaaaaagtggcaccgagtcggtggtgcgccccaggcatcaaataaaacgaaaggctcagtcgaaagactgggcctttcgttttatctgttgtttgtcggtgaacgctctctactagagtcacactggctcaccttcgggtgggcctttctgcgtttatagcccacagctaacaccacgtcgtccctatctgctgccctaggtctatgagtggttgctggataactttacgggcatgcataaggctcgtaatatatattcagggagaccacaacggtttccctctacaaataattttgtttaacttttcacacaggaaacctactagatgagtattcaacatttccgtgtcgcccttattcccttttttgcggcattttgccttcctgtttttgctcacccagaaacgctggtgaaagtaaaagatgccgaagatcagttgggtgcacgtgtgggttacatcgaactggacctcaacagcggtaagattcttgagagttttcgccccgaagaacgtttcccaatgatgagcacttttaaagttctgctctgtggcgcggtattatcccgtattgacgccgggcaagagcaactcggtcgccgcatacactattctcagaatgacttggttgagtactcaccagtcacagaaaagcatcttacggacggcatgacagtacgcgaattatgcagcgctgccataaccatgagtgataacacggcggccaacttacttctgacaacgatcggaggaccgaaggagcttaccgcttttttgcacaacatgggtgatcatgtaactcgccttgatcgttgggaaccggagctgaatgaagccataccaaacgacgagcgtgacaccacgatgcctgtagctatggcaacaacgttgcgcaaactcttaactggcgaacttcttactctcgcttcccggcaacaattaatagactggatggaggcggataaagttgcaggaccacttctgcgctcggcccttccggctggctggtttattgctgataaatctggagccggtgagcgtgggtcccgcggtattattgcagccctggggccagatggtaagccctcccgtatcgtagttatctacacgacggggagccaggcaactatggacgaacgtaatcgccagatcgctgagataggtgcctccctgattaagcattggtaagcccttccgcttcctcgctcactgactcgctacgctcggtcgttcgactgcggcgagcggtgtcagctcactcaaaagcggtaatacggttatccacagaatcaggggataaagccggaaagaacatgtgagcaaaaagcaaagcaccggaagaagccaacgccgcaggcgtttttccataggctccgcccccctgacgagcatcacaaaaatcgacgctcaagccagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgttggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccattggtaactgatttagaggactttgtcttgaagttatgcacctgttaaggctaaactgaaagaacagattttggtgagtgcggtcctccaacccacttaccttggttcaaagagttggtagctcagcgaaccttgagaaaaccaccgttggtagcggtggtttttctttatttatgagatgatgaatcaatcggtctatcaagtcaacgaacagctattccgttgcc

SEQ ID NO: 12 - DNA, 2173 bp, Unknown
  Synthetic
maattgtgagcggataacaattacgagcttcatgcacagtgaaatcatgaaaaatttatttgctttgtgagcggataacaattataatatgtggaattgtgagcgctcacaattccacaacggtttccctctagaaataattttgtttaacttttgccctataaccagaccgttcagcgttttagagctagaaatagcaagttaaaataaggctagtccgttatcaacttgaaaaagtggcaccgagtcggtggtgcgccccaggcatcaaataaaacgaaaggctcagtcgaaagactgggcctttcgttttatctgttgtttgtcggtgaacgctctctactagagtcacactggctcaccttcgggtgggcctttctgcgtttatagcccacagctaacaccacgtcgtccctatctgctgccctaggtctatgagtggttgctggataactttacgggcatgcataaggctcgtaatatatattcagggagaccacaacggtttccctctacaaataattttgtttaacttttcacacaggaaacctactagatgagtattcaacatttccgtgtcgcccttattcccttttttgcggcattttgccttcctgtttttgctcacccagaaacgctggtgaaagtaaaagatgccgaagatcagttgggtgcacgtgtgggttacatcgaactggacctcaacagcggtaagattcttgagagttttcgccccgaagaacgtttcccaatgatgagcacttttaaagttctgctctgtggcgcggtattatcccgtattgacgccgggcaagagcaactcggtcgccgcatacactattctcagaatgacttggttgagtactcaccagtcacagaaaagcatcttacggacggcatgacagtacgcgaattatgcagcgctgccataaccatgagtgataacacggcggccaacttacttctgacaacgatcggaggaccgaaggagcttaccgcttttttgcacaacatgggtgatcatgtaactcgccttgatcgttgggaaccggagctgaatgaagccataccaaacgacgagcgtgacaccacgatgcctgtagctatggcaacaacgttgcgcaaactcttaactggcgaacttcttactctcgcttcccggcaacaattaatagactggatggaggcggataaagttgcaggaccacttctgcgctcggcccttccggctggctggtttattgctgataaatctggagccggtgagcgtgggtcccgcggtattattgcagccctggggccagatggtaagccctcccgtatcgtagttatctacacgacggggagccaggcaactatggacgaacgtaatcgccagatcgctgagataggtgcctccctgattaagcattggtaagcccttccgcttcctcgctcactgactcgctacgctcggtcgttcgactgcggcgagcggtgtcagctcactcaaaagcggtaatacggttatccacagaatcaggggataaagccggaaagaacatgtgagcaaaaagcaaagcaccggaagaagccaacgccgcaggcgtttttccataggctccgcccccctgacgagcatcacaaaaatcgacgctcaagccagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgttggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccattggtaactgatttagaggactttgtcttgaagttatgcacctgttaaggctaaactgaaagaacagattttggtgagtgcggtcctccaacccacttaccttggttcaaagagttggtagctcagcgaaccttgagaaaaccaccgttggtagcggtggtttttctttatttatgagatgatgaatcaatcggtctatcaagtcaacgaacagctattccgttgcc

SEQ ID NO: 13 - DNA, 2448 bp, Unknown
  Synthetic
maattgtgagcggataacaattacgagcttcatgcacagtgaaatcatgaaaaatttatttgctttgtgagcggataacaattataatatgtggaattgtgagcgctcacaattccacaacggtttccctctagaaataattttgtttaacttttgccgtgaataccacgacgatttcgttttagagctagaaatagcaagttaaaataaggctagtccgttatcaacttgaaaaagtggcaccgagtcggtggtgcgccccaggcatcaaataaaacgaaaggctcagtcgaaagactgggcctttcgttttatctgttgtttgtcggtgaacgctctctactagagtcacactggctcaccttcgggtgggcctttctgcgtttatagccttagttagttagattagcagaaagtcaaaagcctccgaccggaggcttttgactaaaacttcccttggggttatcattggggctcactcaaaggcggtaatcagataaaaaaaatccttagctttcgctaaggatgatttctgcccttccgcttcctcgctcactgactcgctacgctcggtcgttcgactgcggcgagcggtgtcagctcactcaaaagcggtaatacggttatccacagaatcaggggataaagccggaaagaacatgtgagcaaaaagcaaagcaccggaagaagccaacgccgcaggcgtttttccataggctccgcccccctgacgagcatcacaaaaatcgacgctcaagccagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgttggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccattggtaactgatttagaggactttgtcttgaagttatgcacctgttaaggctaaactgaaagaacagattttggtgagtgcggtcctccaacccacttaccttggttcaaagagttggtagctcagcgaaccttgagaaaaccaccgttggtagcggtggtttttctttatttatgagatgatgaatcaatcggtctatcaagtcaacgaacagctattccgttgccctgcgctagcatgcctatttgtttatttttctaaatacattcaaatatgtatccgctcatgagacaataaccctgataaatgcttcaataatattgaaaaaggaacagtatgagtattcaacatttccgtgtcgcccttattcccttttttgcggcattttgccttcctgtttttgctcacccagaaacgctggtgaaagtaaaagatgccgaagatcagttgggtgcacgtgtgggttacatcgaactggacctcaacagcggtaagattcttgagagttttcgccccgaagaacgtttcccaatgatgagcacttttaaagttctgctctgtggcgcggtattatcccgtattgacgccgggcaagagcaactcggtcgccgcatacactattctcagaatgacttggttgagtactcaccagtcacagaaaagcatcttacggacggcatgacagtacgcgaattatgcagcgctgccataaccatgagtgataacacggcggccaacttacttctgacaacgatcggaggaccgaaggagcttaccgcttttttgcacaacatgggtgatcatgtaactcgccttgatcgttgggaaccggagctgaatgaagccataccaaacgacgagcgtgacaccacgatgcctgtagctatggcaacaacgttgcgcaaactcttaactggcgaacttcttactctcgcttcccggcaacaattaatagactggatggaggcggataaagttgcaggaccacttctgcgctcggcccttccggctggctggtttattgctgataaatctggagccggtgagcgtgggtcccgcggtattattgcagccctggggccagatggtaagccctcccgtatcgtagttatctacacgacggggagccaggcaactatggacgaacgtaatcgccagatcgctgagataggtgcctccctgattaagcattggtaagcctccggcaaaaaaacgggcaaggtgtcaccaccctgccctttttctttaaaaccgaaaagattacttcgcgtttgccacctgacgtctaagaaaaggaatattcagcaatttgcccgtgccgaagaaaggcccacccgtgaaggtgagccagtgagttgattgctacgtaattagttagttaggcc

SEQ ID NO: 14 - DNA, 2337 bp, Unknown
  Synthetic
mtccctatcagtgatagagattgacatccctatcagtgatagagatactgagcacgccgggcacgggcagcttaccgggttttagagctagaaatagcaagttaaaataaggctagtccgttatcaacttgaaaaagtggcaccgagtcggtggtgcgccccaggcatcaaataaaacgaaaggctcagtcgaaagactgggcctttcgttttatctgttgtttgtcggtgaacgctctctactagagtcacactggctcaccttcgggtgggcctttctgcgtttatagccttagttagttagattagcagaaagtcaaaagcctccgaccggaggcttttgactaaaacttcccttggggttatcattggggctcactcaaaggcggtaatcagataaaaaaaatccttagctttcgctaaggatgatttctgccgatcaaaggatcttcttgagatcctttttttctgcgcgtaatcttttgccctgtaaacgaaaaaaccacctggggaggtggtttgatcgaaggttaagtcagttggggaactgcttaaccgtggtaactggctttcgcagagcacagcaaccaaatctgtccttccagtgtagccggactttggcgcacacttcaagagcaaccgcgtgtttagctaaacaaatcctctgcgaactcccagttaccaatggctgctgccagtggcgttttaccgtgcttttccgggttggactcaagtgaacagttaccggataaggcgcagcagtcgggctgaacggggagttcttgcttacagcccagcttggagcgaacgacctacaccgagccgagataccagtgtgtgagctatgagaaagcgccacacttcccgtaagggagaaaggcggaacaggtatccggtaaacggcagggtcggaacaggagagcgcaagagggagcgacccgccggaaacggtggggatctttaagtcctgtcgggtttcgcccgtactgtcagattcatggttgagcctcacggctcccacagatgcaccggaaaagcgtctgtttatgtgaactctggcaggagggcggagcctatggaaaaacgccaccggcgcggccctgctgttttgcctcacatgttagtcccctgcttatccacggaatctgtgggtaactttgtatgtgtccgcagcgcgccctgcgctagcatgcctatttgtttatttttctaaatacattcaaatatgtatccgctcatgagacaataaccctgataaatgcttcaataatattgaaaaaggaacagtatgagtattcaacatttccgtgtcgcccttattcccttttttgcggcattttgccttcctgtttttgctcacccagaaacgctggtgaaagtaaaagatgccgaagatcagttgggtgcacgtgtgggttacatcgaactggacctcaacagcggtaagattcttgagagttttcgccccgaagaacgtttcccaatgatgagcacttttaaagttctgctctgtggcgcggtattatcccgtattgacgccgggcaagagcaactcggtcgccgcatacactattctcagaatgacttggttgagtactcaccagtcacagaaaagcatcttacggacggcatgacagtacgcgaattatgcagcgctgccataaccatgagtgataacacggcggccaacttacttctgacaacgatcggaggaccgaaggagcttaccgcttttttgcacaacatgggtgatcatgtaactcgccttgatcgttgggaaccggagctgaatgaagccataccaaacgacgagcgtgacaccacgatgcctgtagctatggcaacaacgttgcgcaaactcttaactggcgaacttcttactctcgcttcccggcaacaattaatagactggatggaggcggataaagttgcaggaccacttctgcgctcggcccttccggctggctggtttattgctgataaatctggagccggtgagcgtgggtcccgcggtattattgcagccctggggccagatggtaagccctcccgtatcgtagttatctacacgacggggagccaggcaactatggacgaacgtaatcgccagatcgctgagataggtgcctccctgattaagcattggtaagcctccggcaaaaaaacgggcaaggtgtcaccaccctgccctttttctttaaaaccgaaaagattacttcgcgtttgccacctgacgtctaagaaaaggaatattcagcaatttgcccgtgccgaagaaaggcccacccgtgaaggtgagccagtgagttgattgctacgtaattagttagttaggcc

SEQ ID NO: 15 - DNA, 2337 bp, Unknown
  Synthetic
mtccctatcagtgatagagattgacatccctatcagtgatagagatactgagcacgccgtgaataccacgacgatttcgttttagagctagaaatagcaagttaaaataaggctagtccgttatcaacttgaaaaagtggcaccgagtcggtggtgcgccccaggcatcaaataaaacgaaaggctcagtcgaaagactgggcctttcgttttatctgttgtttgtcggtgaacgctctctactagagtcacactggctcaccttcgggtgggcctttctgcgtttatagccttagttagttagattagcagaaagtcaaaagcctccgaccggaggcttttgactaaaacttcccttggggttatcattggggctcactcaaaggcggtaatcagataaaaaaaatccttagctttcgctaaggatgatttctgccgatcaaaggatcttcttgagatcctttttttctgcgcgtaatcttttgccctgtaaacgaaaaaaccacctggggaggtggtttgatcgaaggttaagtcagttggggaactgcttaaccgtggtaactggctttcgcagagcacagcaaccaaatctgtccttccagtgtagccggactttggcgcacacttcaagagcaaccgcgtgtttagctaaacaaatcctctgcgaactcccagttaccaatggctgctgccagtggcgttttaccgtgcttttccgggttggactcaagtgaacagttaccggataaggcgcagcagtcgggctgaacggggagttcttgcttacagcccagcttggagcgaacgacctacaccgagccgagataccagtgtgtgagctatgagaaagcgccacacttcccgtaagggagaaaggcggaacaggtatccggtaaacggcagggtcggaacaggagagcgcaagagggagcgacccgccggaaacggtggggatctttaagtcctgtcgggtttcgcccgtactgtcagattcatggttgagcctcacggctcccacagatgcaccggaaaagcgtctgtttatgtgaactctggcaggagggcggagcctatggaaaaacgccaccggcgcggccctgctgttttgcctcacatgttagtcccctgcttatccacggaatctgtgggtaactttgtatgtgtccgcagcgcgccctgcgctagcatgcctatttgtttatttttctaaatacattcaaatatgtatccgctcatgagacaataaccctgataaatgcttcaataatattgaaaaaggaacagtatgagtattcaacatttccgtgtcgcccttattcccttttttgcggcattttgccttcctgtttttgctcacccagaaacgctggtgaaagtaaaagatgccgaagatcagttgggtgcacgtgtgggttacatcgaactggacctcaacagcggtaagattcttgagagttttcgccccgaagaacgtttcccaatgatgagcacttttaaagttctgctctgtggcgcggtattatcccgtattgacgccgggcaagagcaactcggtcgccgcatacactattctcagaatgacttggttgagtactcaccagtcacagaaaagcatcttacggacggcatgacagtacgcgaattatgcagcgctgccataaccatgagtgataacacggcggccaacttacttctgacaacgatcggaggaccgaaggagcttaccgcttttttgcacaacatgggtgatcatgtaactcgccttgatcgttgggaaccggagctgaatgaagccataccaaacgacgagcgtgacaccacgatgcctgtagctatggcaacaacgttgcgcaaactcttaactggcgaacttcttactctcgcttcccggcaacaattaatagactggatggaggcggataaagttgcaggaccacttctgcgctcggcccttccggctggctggtttattgctgataaatctggagccggtgagcgtgggtcccgcggtattattgcagccctggggccagatggtaagccctcccgtatcgtagttatctacacgacggggagccaggcaactatggacgaacgtaatcgccagatcgctgagataggtgcctccctgattaagcattggtaagcctccggcaaaaaaacgggcaaggtgtcaccaccctgccctttttctttaaaaccgaaaagattacttcgcgtttgccacctgacgtctaagaaaaggaatattcagcaatttgcccgtgccgaagaaaggcccacccgtgaaggtgagccagtgagttgattgctacgtaattagttagttaggcc

SEQ ID NO: 16 - DNA, 2337 bp, Unknown
  Synthetic
mtccctatcagtgatagagattgacatccctatcagtgatagagatactgagcacgccctataaccagaccgttcagcgttttagagctagaaatagcaagttaaaataaggctagtccgttatcaacttgaaaaagtggcaccgagtcggtggtgcgccccaggcatcaaataaaacgaaaggctcagtcgaaagactgggcctttcgttttatctgttgtttgtcggtgaacgctctctactagagtcacactggctcaccttcgggtgggcctttctgcgtttatagccttagttagttagattagcagaaagtcaaaagcctccgaccggaggcttttgactaaaacttcccttggggttatcattggggctcactcaaaggcggtaatcagataaaaaaaatccttagctttcgctaaggatgatttctgccgatcaaaggatcttcttgagatcctttttttctgcgcgtaatcttttgccctgtaaacgaaaaaaccacctggggaggtggtttgatcgaaggttaagtcagttggggaactgcttaaccgtggtaactggctttcgcagagcacagcaaccaaatctgtccttccagtgtagccggactttggcgcacacttcaagagcaaccgcgtgtttagctaaacaaatcctctgcgaactcccagttaccaatggctgctgccagtggcgttttaccgtgcttttccgggttggactcaagtgaacagttaccggataaggcgcagcagtcgggctgaacggggagttcttgcttacagcccagcttggagcgaacgacctacaccgagccgagataccagtgtgtgagctatgagaaagcgccacacttcccgtaagggagaaaggcggaacaggtatccggtaaacggcagggtcggaacaggagagcgcaagagggagcgacccgccggaaacggtggggatctttaagtcctgtcgggtttcgcccgtactgtcagattcatggttgagcctcacggctcccacagatgcaccggaaaagcgtctgtttatgtgaactctggcaggagggcggagcctatggaaaaacgccaccggcgcggccctgctgttttgcctcacatgttagtcccctgcttatccacggaatctgtgggtaactttgtatgtgtccgcagcgcgccctgcgctagcatgcctatttgtttatttttctaaatacattcaaatatgtatccgctcatgagacaataaccctgataaatgcttcaataatattgaaaaaggaacagtatgagtattcaacatttccgtgtcgcccttattcccttttttgcggcattttgccttcctgtttttgctcacccagaaacgctggtgaaagtaaaagatgccgaagatcagttgggtgcacgtgtgggttacatcgaactggacctcaacagcggtaagattcttgagagttttcgccccgaagaacgtttcccaatgatgagcacttttaaagttctgctctgtggcgcggtattatcccgtattgacgccgggcaagagcaactcggtcgccgcatacactattctcagaatgacttggttgagtactcaccagtcacagaaaagcatcttacggacggcatgacagtacgcgaattatgcagcgctgccataaccatgagtgataacacggcggccaacttacttctgacaacgatcggaggaccgaaggagcttaccgcttttttgcacaacatgggtgatcatgtaactcgccttgatcgttgggaaccggagctgaatgaagccataccaaacgacgagcgtgacaccacgatgcctgtagctatggcaacaacgttgcgcaaactcttaactggcgaacttcttactctcgcttcccggcaacaattaatagactggatggaggcggataaagttgcaggaccacttctgcgctcggcccttccggctggctggtttattgctgataaatctggagccggtgagcgtgggtcccgcggtattattgcagccctggggccagatggtaagccctcccgtatcgtagttatctacacgacggggagccaggcaactatggacgaacgtaatcgccagatcgctgagataggtgcctccctgattaagcattggtaagcctccggcaaaaaaacgggcaaggtgtcaccaccctgccctttttctttaaaaccgaaaagattacttcgcgtttgccacctgacgtctaagaaaaggaatattcagcaatttgcccgtgccgaagaaaggcccacccgtgaaggtgagccagtgagttgattgctacgtaattagttagttaggcc

SEQ ID NO: 17 - DNA, 13367 bp, Unknown
  Synthetic
aattgtgagcggataacaattacgagcttcatgcacagtgaaatcatgaaaaatttatttgctttgtgagcggataacaattataatatgtggaattgtgagcgctcacaattccacaacggtttccctctagaaataattttgtttaacttttgcccaccggggtggtgcccatccgttttagagctagaaatagcaagttaaaataaggctagtccgttatcaacttgaaaaagtggcaccgagtcggtggtgcgccccaggcatcaaataaaacgaaaggctcagtcgaaagactgggcctttcgttttatctgttgtttgtcggtgaacgctctctactagagtcacactggctcaccttcgggtgggcctttctgcgtttatagcccacagcaaacaccacgtcgaccctatcagctgcgtgctttctatgagtcgttgctgcataacttgacaattaatcatccggctcgtaatgtttgtggagccgggcccaagttcacttaaaaaggagatcaacaatgaaagcaattttcgtactgaaacatcttaatcatgctaaggaggttttctaatggtaagcaagggcgaggagaataacatggccatcatcaaggagttcatgcgcttcaaggtgcacatggagggcagtgtgaacggccacgagttcgagatcgagggcgagggcgagggccgtccctacgaggcctttcagaccgctaagctgaaggtgaccaagggtggccccctgcccttcgcctgggacatcctgtcccctcagttcatgtacggctccaaggtctacattaagcacccagccgacatccccgactacttcaagctgtccttccccgagggcttcaggtgggagcgcgtgatgaacttcgaggacggcggcattattcacgttaaccaggacagttccctgcaagacggcgtgttcatctacaaggtgaagctgcgcggcaccaacttccccagtgacggccccgtaatgcagaagaagaccatgggctgggaggccagtgaggagcggatgtaccccgaggacggcgccctgaagtctgagatcaaaaagaggctgaagctgaaggacggcggccactacgccgccgaggtcaagaccacctacaaggccaagaagcccgtgcagctgcccggcgcctacatcgtcgacatcaagttggacatcgtgtcccacaacgaggactacaccatcgtggaacagtacgaacgcgccgagggccgccactccaccggcggcatggacgagctgtacaaggccgacaagaaatactcgatagggctggatatcgggaccaactccgtcggttgggctgttatcacggatgaatataaggtgcccagcaaaaagttcaaggtgctaggtaacaccgaccggcacagtatcaaaaaaaacttgataggagcgttgctgtttgacagtggtgagaccgctgaagctacgcgccttaaaaggaccgcgcgccgtagatatacccgcaggaagaaccgcatttgctatctccaagagattttttcgaacgagatggctaaagtagatgatagtttctttcatcgattggaagaaagctttttagttgaagaagacaagaagcatgaacgccatccaatttttggtaacatagtagatgaagtggcgtatcatgagaaatatccgacgatctatcatctacgaaaaaaattagtagatagcacggataaagctgatttacggctgatctatttagcactggctcatatgattaagtttcgcggccattttttaattgagggtgatctgaacccagataacagtgatgttgacaaactctttatccaattagtacagacttacaaccagctgtttgaagaaaatccaattaatgccagtggtgtagatgcgaaagctattttgagcgcccgattaagtaaatcgcgtcgactggaaaaccttattgcgcaacttcctggcgagaagaaaaacgggctgtttggaaaccttattgcgttatcgttaggcttaactccaaactttaaatcgaactttgatttagccgaagatgcgaaactgcaattgtcgaaagatacgtacgatgatgatctggataacctgttagctcagattggtgatcagtatgcggatttatttttagccgcgaagaacctgtcggatgcgattctgttgtcggatatcctccgtgtaaacacggaaataacgaaggcgcctctctcggcgtcgatgattaaacggtacgatgaacatcatcaggacttaacgttgctgaaagcgctggtgcgacagcagttgccggaaaagtataaagaaatcttttttgatcagtcgaaaaatggttatgccggctatattgatggaggtgcgtcccaggaagaattttataaatttatcaaaccgattctggaaaaaatggatggcacggaggaactgttggttaaactcaaccgcgaagatttgctacggaagcagaggacttttgacaatgggagcattcctcatcagattcacttgggcgagctacatgcgattttgcgtcgtcaggaagacttttatccgtttctgaaagacaaccgcgagaagattgaaaaaatcttgacgtttcgaatcccatattatgtgggcccgttagctcgcgggaacagtcgctttgcctggatgacgaggaagtcggaagaaaccattactccgtggaactttgaagaagtggtcgataaaggcgcgtcggcgcagtcgtttattgaacggatgaccaattttgataaaaacttgccgaacgaaaaagtactcccgaaacatagtttattgtatgagtattttacagtgtataatgaattaaccaaggtcaaatatgtgacggaaggtatgcgaaaaccggcctttttgtcgggcgaacaaaagaaagcaattgtggatctgcttttcaaaaccaaccgaaaagtaactgtgaagcagctgaaagaagattatttcaaaaaaatagaatgctttgatagtgtggaaatttcgggtgtggaagatcgttttaacgcgtcgctgggcacttaccatgatttactcaaaattattaaagataaagattttttagataacgaagaaaacgaagatatcctggaggatattgtgctgaccttaactctgtttgaagatagagagatgattgaggaacgtttgaaaacctatgcgcacctttttgatgataaggttatgaaacaattgaaacgccggcgctatacgggctggggtcgcttaagccgaaaattaattaacggcattagagataagcagagcgggaaaaccatactggattttttaaaatcggatggctttgcaaaccggaactttatgcaactaatccatgatgatagtttaacctttaaagaagacattcagaaagcccaggttagcggtcagggggatagtctgcatgaacatattgccaacctggcgggctccccagcgattaaaaaaggcattctgcaaacggtaaaagtggtggatgaattagtcaaagtaatgggaaggcataagccggaaaacatcgtgattgaaatggcccgcgaaaaccaaaccacgcagaaggggcaaaaaaactcacgagagcgcatgaaacgaatcgaagaaggcatcaaagaactgggtagtcaaattttgaaagagcatccagtggaaaacacgcagttacagaacgaaaagctttatctttattatcttcagaacggtcgtgacatgtatgttgaccaggaactggatattaaccgcctgagtgattatgatgtcgatcacattgtgccgcagagtttcttgaaagacgattcgatagacaacaaggtcctgacacgcagcgataaaaaccgcggcaaatcagataatgtgccgagtgaagaagtagtcaaaaagatgaaaaattattggcgtcagttgctcaatgcaaagctgatcacgcagcgcaagtttgataacctgacaaaagcggaacgcggtggcttaagtgaattggataaagcgggctttatcaaacggcagttagtggaaacgcggcagatcacgaagcatgttgcccagattttagatagtcggatgaacacgaaatacgatgaaaacgataaattgattcgagaggtgaaagttattactctgaaaagcaaactggtgagcgacttccgaaaagatttccagttctataaagtacgcgagattaataactaccatcatgcacatgatgcttatctcaacgcagtcgtgggtacggcgttaattaagaaatatccgaaattggaatcagagtttgtctatggcgattataaagtgtatgatgtgcgcaaaatgattgcgaagtcggagcaggaaatagggaaagccactgccaaatatttcttttacagcaacatcatgaatttcttcaaaaccgaaattaccttggccaacggtgagattcggaaacggccactcatcgaaacgaacggagaaacgggtgaaattgtctgggataaaggacgagattttgcaaccgttcggaaagtattgtccatgcctcaggtcaacattgtcaagaaaaccgaagtacaaaccgggggtttctccaaggagtcgattctgccgaaacgcaactcagacaagttgattgcgcgcaaaaaagactgggatccgaaaaaatatggcggcttcgattccccgacagtagcgtactcggtcctcgtcgtggcgaaggtcgaaaaagggaaatccaagaagctgaaatccgtgaaagagctgctcgggatcaccatcatggagcgctcctccttcgagaagaaccccatcgacttcctggaggcgaagggctacaaggaggtgaagaaggacctgatcatcaagctccccaagtactccctctttgagctggagaacggccgcaagaggatgctcgcaagtgcaggtgaattacaaaaaggtaatgaactagcgctaccgtccaaatatgttaactttctgtatctggcgagtcattatgaaaagttaaagggcagtccggaagataatgaacagaaacagttatttgttgagcaacataagcattatctggatgagattattgagcagatcagtgaatttagcaagcgcgtgattctggccgatgcaaacctggataaagtgttgagtgcctataataaacatcgtgacaaaccgatacgcgaacaggccgaaaacattattcatctgtttacattaacaaacttgggtgcgcctgcggcgtttaaatattttgataccaccattgatcgcaaacgatatacaagcaccaaagaagtgctggatgcaacgttgatccatcagtctatcacgggcttgtatgaaacccggattgatttaagtcaactcggtggcgactaagcccggccgctactagtaacaaaaaacccctagccccccgttttatatcactgcccgctttccagtcgggaaacctgtcgtgccagctgcattaatgaatcggccaacgcgcggggagaggcggtttgcgtattgggcgccagggtggtttttcttttcaccagtgagactggcaacagctgattgcccttcaccgcctggccctgagagagttgcagcaagcggtccacgctggtttgccccagcaggcgaaaatcctgtttgatggtggttaacggcgggatataacatgagctatcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcctcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcatattcaccaccctgaattgactgtcttccgggcgctatcatgccataccgcgaaaggttttgcgccattcgatggcgcgccgcgccctgcagcccgggggatccactagttctagagcggccgccaccgcggtggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgggtaccgggccccccctcgaggtcgacggatcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgatcccccgataagcttcgacggtatcgataagcttcgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcaagcttgacggaagccgatactgcatcttcatggtttcaccgccgacacagccagccggggagtgacggctcacaggtgccgctccgcgtcctgtgcgctatgcaggacgcggacaatctccagccagtctccccggtcgcggaaatagatcacatgcgagccggtggggcatttgcgatagccaaggcgaacattcgtcgcccgcccctgcttccagcccgatgccagcgcatggcaggcgtcccggatttcgtcggtgtagcggtcggcttgatctggtccccagttctcagcgctgtaatcccagataccgtcaatgtcggcttctgcccgaggcgagaaggaaagagccttcatctgccctcatactccgcgcgcttccgcttcttgaatgcctcgaagtcaaagggctgcggttcgccggattcctcgccttcgatcaacgccgcttccagcgcctttaccttggcctcatgctcttgcagcagccgcagccctgcccgcaccacatcgctggccgatccgtagcgcccggcctgcacctgcgtgtcaatgaacgtggtgaaatggtccccgagggacacggacgtattgcgggccatcgccgcccccttcctgttctaagcagattcccaaaatataccaataagtaccaagaggcaatctggctgctgtcggtgccagcggtcgcgcttccgcgacggcaagcggcgcagggcgcttcgcaccctgcgcgcaccgcacccgtcttacccttttgggtaagggtcttgatcctcggaattggcacaggctctagtgttattagtgttacctacggagtaagttacgtcccccataagcatctgtgaaaaatggcataaaccggtattttggttccgaaagggtcaaactttggttccgaaagggtcaaactttggttccgaaagggtcaaactgcctttccatcgacaaccgttcctgataggtcaaacaagatagttcctgataggtcaaagttgcgccgtatgagcctgcttccagatcggcacccaaacctagatttcttcgtgctcgacatcgcggatgcggtgccgaaggatgacatggcgtcgatggagcatccgctgttttcgttggcaaccaagccggacatgcggcacctagaatatcgtaacggcgacaacgttctgaaaatccgaccctctgggctcggcctgccaacgatcttcgacaaggacattctaatcttcaccatcagccagttgatggcccggaaaaatcgtggcgaaccgattggcgacacggtccgcttctcggcgcgagagctgtctgttgcgacgaaccggcctatcggtgggaaccactacaagcgccttgaggatgccttcgcccgcctgcaaggggcgcagttcgtcaccaacatcaagtccggggggaaaatcgaaacccggatattctccctgatcgacgagggtggttttgtccgcactgacgatgagcgattccgcctcgactactgcgaggtcaagctgtcgcgctggctgatgcgggcgatagagactgaccaggtggtgacgatcacccatgactacttccgcctgcgcaggcccttggagcgccgcctgtatgagatcgcccgtaagcactgcggcaactccccgaagtggcagatcggacttgcaaacctccagaacaagacaggcagcaatgccccggcaaagcgctttcgccacaacctgcgcgagatcatgcgagcagatgtgacaccgttctataagttcaatatcgacgaaaacgacatggtgacagtgcgccctcgctctacgccggtcgatccttcttcaccaatcatcatcccagaatgggccgaggagcaggccagagcacacgctcgaaccctcggctgggattactacaccctacggtctaactggatggcatacgctcacgaggaaaccgccaagggcaaccccccgaaaaatcccggcgcggcatttgtggcatactgcaaaaaacaagagaagctgcgctgacagcgcaaggactggacgcgaacaggcgaacagtccacgaattcgcc

SEQ ID NO: 18 - DNA, 13367 bp, unknown
  Synthetic
aattgtgagcggataacaattacgagcttcatgcacagtgaaatcatgaaaaatttatttgctttgtgagcggataacaattataatatgtggaattgtgagcgctcacaattccacaacggtttccctctagaaataattttgtttaacttttgccctataaccagaccgttcagcgttttagagctagaaatagcaagttaaaataaggctagtccgttatcaacttgaaaaagtggcaccgagtcggtggtgcgccccaggcatcaaataaaacgaaaggctcagtcgaaagactgggcctttcgttttatctgttgtttgtcggtgaacgctctctactagagtcacactggctcaccttcgggtgggcctttctgcgtttatagcccacagcaaacaccacgtcgaccctatcagctgcgtgctttctatgagtcgttgctgcataacttgacaattaatcatccggctcgtaatgtttgtggagccgggcccaagttcacttaaaaaggagatcaacaatgaaagcaattttcgtactgaaacatcttaatcatgctaaggaggttttctaatggtaagcaagggcgaggagaataacatggccatcatcaaggagttcatgcgcttcaaggtgcacatggagggcagtgtgaacggccacgagttcgagatcgagggcgagggcgagggccgtccctacgaggcctttcagaccgctaagctgaaggtgaccaagggtggccccctgcccttcgcctgggacatcctgtcccctcagttcatgtacggctccaaggtctacattaagcacccagccgacatccccgactacttcaagctgtccttccccgagggcttcaggtgggagcgcgtgatgaacttcgaggacggcggcattattcacgttaaccaggacagttccctgcaagacggcgtgttcatctacaaggtgaagctgcgcggcaccaacttccccagtgacggccccgtaatgcagaagaagaccatgggctgggaggccagtgaggagcggatgtaccccgaggacggcgccctgaagtctgagatcaaaaagaggctgaagctgaaggacggcggccactacgccgccgaggtcaagaccacctacaaggccaagaagcccgtgcagctgcccggcgcctacatcgtcgacatcaagttggacatcgtgtcccacaacgaggactacaccatcgtggaacagtacgaacgcgccgagggccgccactccaccggcggcatggacgagctgtacaaggccgacaagaaatactcgatagggctggatatcgggaccaactccgtcggttgggctgttatcacggatgaatataaggtgcccagcaaaaagttcaaggtgctaggtaacaccgaccggcacagtatcaaaaaaaacttgataggagcgttgctgtttgacagtggtgagaccgctgaagctacgcgccttaaaaggaccgcgcgccgtagatatacccgcaggaagaaccgcatttgctatctccaagagattttttcgaacgagatggctaaagtagatgatagtttctttcatcgattggaagaaagctttttagttgaagaagacaagaagcatgaacgccatccaatttttggtaacatagtagatgaagtggcgtatcatgagaaatatccgacgatctatcatctacgaaaaaaattagtagatagcacggataaagctgatttacggctgatctatttagcactggctcatatgattaagtttcgcggccattttttaattgagggtgatctgaacccagataacagtgatgttgacaaactctttatccaattagtacagacttacaaccagctgtttgaagaaaatccaattaatgccagtggtgtagatgcgaaagctattttgagcgcccgattaagtaaatcgcgtcgactggaaaaccttattgcgcaacttcctggcgagaagaaaaacgggctgtttggaaaccttattgcgttatcgttaggcttaactccaaactttaaatcgaactttgatttagccgaagatgcgaaactgcaattgtcgaaagatacgtacgatgatgatctggataacctgttagctcagattggtgatcagtatgcggatttatttttagccgcgaagaacctgtcggatgcgattctgttgtcggatatcctccgtgtaaacacggaaataacgaaggcgcctctctcggcgtcgatgattaaacggtacgatgaacatcatcaggacttaacgttgctgaaagcgctggtgcgacagcagttgccggaaaagtataaagaaatcttttttgatcagtcgaaaaatggttatgccggctatattgatggaggtgcgtcccaggaagaattttataaatttatcaaaccgattctggaaaaaatggatggcacggaggaactgttggttaaactcaaccgcgaagatttgctacggaagcagaggacttttgacaatgggagcattcctcatcagattcacttgggcgagctacatgcgattttgcgtcgtcaggaagacttttatccgtttctgaaagacaaccgcgagaagattgaaaaaatcttgacgtttcgaatcccatattatgtgggcccgttagctcgcgggaacagtcgctttgcctggatgacgaggaagtcggaagaaaccattactccgtggaactttgaagaagtggtcgataaaggcgcgtcggcgcagtcgtttattgaacggatgaccaattttgataaaaacttgccgaacgaaaaagtactcccgaaacatagtttattgtatgagtattttacagtgtataatgaattaaccaaggtcaaatatgtgacggaaggtatgcgaaaaccggcctttttgtcgggcgaacaaaagaaagcaattgtggatctgcttttcaaaaccaaccgaaaagtaactgtgaagcagctgaaagaagattatttcaaaaaaatagaatgctttgatagtgtggaaatttcgggtgtggaagatcgttttaacgcgtcgctgggcacttaccatgatttactcaaaattattaaagataaagattttttagataacgaagaaaacgaagatatcctggaggatattgtgctgaccttaactctgtttgaagatagagagatgattgaggaacgtttgaaaacctatgcgcacctttttgatgataaggttatgaaacaattgaaacgccggcgctatacgggctggggtcgcttaagccgaaaattaattaacggcattagagataagcagagcgggaaaaccatactggattttttaaaatcggatggctttgcaaaccggaactttatgcaactaatccatgatgatagtttaacctttaaagaagacattcagaaagcccaggttagcggtcagggggatagtctgcatgaacatattgccaacctggcgggctccccagcgattaaaaaaggcattctgcaaacggtaaaagtggtggatgaattagtcaaagtaatgggaaggcataagccggaaaacatcgtgattgaaatggcccgcgaaaaccaaaccacgcagaaggggcaaaaaaactcacgagagcgcatgaaacgaatcgaagaaggcatcaaagaactgggtagtcaaattttgaaagagcatccagtggaaaacacgcagttacagaacgaaaagctttatctttattatcttcagaacggtcgtgacatgtatgttgaccaggaactggatattaaccgcctgagtgattatgatgtcgatcacattgtgccgcagagtttcttgaaagacgattcgatagacaacaaggtcctgacacgcagcgataaaaaccgcggcaaatcagataatgtgccgagtgaagaagtagtcaaaaagatgaaaaattattggcgtcagttgctcaatgcaaagctgatcacgcagcgcaagtttgataacctgacaaaagcggaacgcggtggcttaagtgaattggataaagcgggctttatcaaacggcagttagtggaaacgcggcagatcacgaagcatgttgcccagattttagatagtcggatgaacacgaaatacgatgaaaacgataaattgattcgagaggtgaaagttattactctgaaaagcaaactggtgagcgacttccgaaaagatttccagttctataaagtacgcgagattaataactaccatcatgcacatgatgcttatctcaacgcagtcgtgggtacggcgttaattaagaaatatccgaaattggaatcagagtttgtctatggcgattataaagtgtatgatgtgcgcaaaatgattgcgaagtcggagcaggaaatagggaaagccactgccaaatatttcttttacagcaacatcatgaatttcttcaaaaccgaaattaccttggccaacggtgagattcggaaacggccactcatcgaaacgaacggagaaacgggtgaaattgtctgggataaaggacgagattttgcaaccgttcggaaagtattgtccatgcctcaggtcaacattgtcaagaaaaccgaagtacaaaccgggggtttctccaaggagtcgattctgccgaaacgcaactcagacaagttgattgcgcgcaaaaaagactgggatccgaaaaaatatggcggcttcgattccccgacagtagcgtactcggtcctcgtcgtggcgaaggtcgaaaaagggaaatccaagaagctgaaatccgtgaaagagctgctcgggatcaccatcatggagcgctcctccttcgagaagaaccccatcgacttcctggaggcgaagggctacaaggaggtgaagaaggacctgatcatcaagctccccaagtactccctctttgagctggagaacggccgcaagaggatgctcgcaagtgcaggtgaattacaaaaaggtaatgaactagcgctaccgtccaaatatgttaactttctgtatctggcgagtcattatgaaaagttaaagggcagtccggaagataatgaacagaaacagttatttgttgagcaacataagcattatctggatgagattattgagcagatcagtgaatttagcaagcgcgtgattctggccgatgcaaacctggataaagtgttgagtgcctataataaacatcgtgacaaaccgatacgcgaacaggccgaaaacattattcatctgtttacattaacaaacttgggtgcgcctgcggcgtttaaatattttgataccaccattgatcgcaaacgatatacaagcaccaaagaagtgctggatgcaacgttgatccatcagtctatcacgggcttgtatgaaacccggattgatttaagtcaactcggtggcgactaagcccggccgctactagtaacaaaaaacccctagccccccgttttatatcactgcccgctttccagtcgggaaacctgtcgtgccagctgcattaatgaatcggccaacgcgcggggagaggcggtttgcgtattgggcgccagggtggtttttcttttcaccagtgagactggcaacagctgattgcccttcaccgcctggccctgagagagttgcagcaagcggtccacgctggtttgccccagcaggcgaaaatcctgtttgatggtggttaacggcgggatataacatgagctatcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcctcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcatattcaccaccctgaattgactgtcttccgggcgctatcatgccataccgcgaaaggttttgcgccattcgatggcgcgccgcgccctgcagcccgggggatccactagttctagagcggccgccaccgcggtggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgggtaccgggccccccctcgaggtcgacggatcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgatcccccgataagcttcgacggtatcgataagcttcgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcaagcttgacggaagccgatactgcatcttcatggtttcaccgccgacacagccagccggggagtgacggctcacaggtgccgctccgcgtcctgtgcgctatgcaggacgcggacaatctccagccagtctccccggtcgcggaaatagatcacatgcgagccggtggggcatttgcgatagccaaggcgaacattcgtcgcccgcccctgcttccagcccgatgccagcgcatggcaggcgtcccggatttcgtcggtgtagcggtcggcttgatctggtccccagttctcagcgctgtaatcccagataccgtcaatgtcggcttctgcccgaggcgagaaggaaagagccttcatctgccctcatactccgcgcgcttccgcttcttgaatgcctcgaagtcaaagggctgcggttcgccggattcctcgccttcgatcaacgccgcttccagcgcctttaccttggcctcatgctcttgcagcagccgcagccctgcccgcaccacatcgctggccgatccgtagcgcccggcctgcacctgcgtgtcaatgaacgtggtgaaatggtccccgagggacacggacgtattgcgggccatcgccgcccccttcctgttctaagcagattcccaaaatataccaataagtaccaagaggcaatctggctgctgtcggtgccagcggtcgcgcttccgcgacggcaagcggcgcagggcgcttcgcaccctgcgcgcaccgcacccgtcttacccttttgggtaagggtcttgatcctcggaattggcacaggctctagtgttattagtgttacctacggagtaagttacgtcccccataagcatctgtgaaaaatggcataaaccggtattttggttccgaaagggtcaaactttggttccgaaagggtcaaactttggttccgaaagggtcaaactgcctttccatcgacaaccgttcctgataggtcaaacaagatagttcctgataggtcaaagttgcgccgtatgagcctgcttccagatcggcacccaaacctagatttcttcgtgctcgacatcgcggatgcggtgccgaaggatgacatggcgtcgatggagcatccgctgttttcgttggcaaccaagccggacatgcggcacctagaatatcgtaacggcgacaacgttctgaaaatccgaccctctgggctcggcctgccaacgatcttcgacaaggacattctaatcttcaccatcagccagttgatggcccggaaaaatcgtggcgaaccgattggcgacacggtccgcttctcggcgcgagagctgtctgttgcgacgaaccggcctatcggtgggaaccactacaagcgccttgaggatgccttcgcccgcctgcaaggggcgcagttcgtcaccaacatcaagtccggggggaaaatcgaaacccggatattctccctgatcgacgagggtggttttgtccgcactgacgatgagcgattccgcctcgactactgcgaggtcaagctgtcgcgctggctgatgcgggcgatagagactgaccaggtggtgacgatcacccatgactacttccgcctgcgcaggcccttggagcgccgcctgtatgagatcgcccgtaagcactgcggcaactccccgaagtggcagatcggacttgcaaacctccagaacaagacaggcagcaatgccccggcaaagcgctttcgccacaacctgcgcgagatcatgcgagcagatgtgacaccgttctataagttcaatatcgacgaaaacgacatggtgacagtgcgccctcgctctacgccggtcgatccttcttcaccaatcatcatcccagaatgggccgaggagcaggccagagcacacgctcgaaccctcggctgggattactacaccctacggtctaactggatggcatacgctcacgaggaaaccgccaagggcaaccccccgaaaaatcccggcgcggcatttgtggcatactgcaaaaaacaagagaagctgcgctgacagcgcaaggactggacgcgaacaggcgaacagtccacgaattcgcc

SEQ ID NO: 19 - DNA, 11077 bp, unknown
  Synthetic
aattgtgagcggataacaattacgagcttcatgcacagtgaaatcatgaaaaatttatttgctttgtgagcggataacaattataatatgtggaattgtgagcgctcacaattccacaacggtttccctctagaaataattttgtttaacttttgcccaccggggtggtgcccatccgttttagagctagaaatagcaagttaaaataaggctagtccgttatcaacttgaaaaagtggcaccgagtcggtggtgcgccccaggcatcaaataaaacgaaaggctcagtcgaaagactgggcctttcgttttatctgttgtttgtcggtgaacgctctctactagagtcacactggctcaccttcgggtgggcctttctgcgtttatagcccacagcaaacaccacgtcgaccctatcagctgcgtgctttctatgagtcgttgctgcataacttgacaattaatcatccggctcgtaatgtttgtggagccgggcccaagttcacttaaaaaggagatcaacaatgaaagcaattttcgtactgaaacatcttaatcatgctaaggaggttttctaatggtaagcaagggcgaggagaataacatggccatcatcaaggagttcatgcgcttcaaggtgcacatggagggcagtgtgaacggccacgagttcgagatcgagggcgagggcgagggccgtccctacgaggcctttcagaccgctaagctgaaggtgaccaagggtggccccctgcccttcgcctgggacatcctgtcccctcagttcatgtacggctccaaggtctacattaagcacccagccgacatccccgactacttcaagctgtccttccccgagggcttcaggtgggagcgcgtgatgaacttcgaggacggcggcattattcacgttaaccaggacagttccctgcaagacggcgtgttcatctacaaggtgaagctgcgcggcaccaacttccccagtgacggccccgtaatgcagaagaagaccatgggctgggaggccagtgaggagcggatgtaccccgaggacggcgccctgaagtctgagatcaaaaagaggctgaagctgaaggacggcggccactacgccgccgaggtcaagaccacctacaaggccaagaagcccgtgcagctgcccggcgcctacatcgtcgacatcaagttggacatcgtgtcccacaacgaggactacaccatcgtggaacagtacgaacgcgccgagggccgccactccaccggcggcatggacgagctgtacaaggccgacaagaaatactcgatagggctggatatcgggaccaactccgtcggttgggctgttatcacggatgaatataaggtgcccagcaaaaagttcaaggtgctaggtaacaccgaccggcacagtatcaaaaaaaacttgataggagcgttgctgtttgacagtggtgagaccgctgaagctacgcgccttaaaaggaccgcgcgccgtagatatacccgcaggaagaaccgcatttgctatctccaagagattttttcgaacgagatggctaaagtagatgatagtttctttcatcgattggaagaaagctttttagttgaagaagacaagaagcatgaacgccatccaatttttggtaacatagtagatgaagtggcgtatcatgagaaatatccgacgatctatcatctacgaaaaaaattagtagatagcacggataaagctgatttacggctgatctatttagcactggctcatatgattaagtttcgcggccattttttaattgagggtgatctgaacccagataacagtgatgttgacaaactctttatccaattagtacagacttacaaccagctgtttgaagaaaatccaattaatgccagtggtgtagatgcgaaagctattttgagcgcccgattaagtaaatcgcgtcgactggaaaaccttattgcgcaacttcctggcgagaagaaaaacgggctgtttggaaaccttattgcgttatcgttaggcttaactccaaactttaaatcgaactttgatttagccgaagatgcgaaactgcaattgtcgaaagatacgtacgatgatgatctggataacctgttagctcagattggtgatcagtatgcggatttatttttagccgcgaagaacctgtcggatgcgattctgttgtcggatatcctccgtgtaaacacggaaataacgaaggcgcctctctcggcgtcgatgattaaacggtacgatgaacatcatcaggacttaacgttgctgaaagcgctggtgcgacagcagttgccggaaaagtataaagaaatcttttttgatcagtcgaaaaatggttatgccggctatattgatggaggtgcgtcccaggaagaattttataaatttatcaaaccgattctggaaaaaatggatggcacggaggaactgttggttaaactcaaccgcgaagatttgctacggaagcagaggacttttgacaatgggagcattcctcatcagattcacttgggcgagctacatgcgattttgcgtcgtcaggaagacttttatccgtttctgaaagacaaccgcgagaagattgaaaaaatcttgacgtttcgaatcccatattatgtgggcccgttagctcgcgggaacagtcgctttgcctggatgacgaggaagtcggaagaaaccattactccgtggaactttgaagaagtggtcgataaaggcgcgtcggcgcagtcgtttattgaacggatgaccaattttgataaaaacttgccgaacgaaaaagtactcccgaaacatagtttattgtatgagtattttacagtgtataatgaattaaccaaggtcaaatatgtgacggaaggtatgcgaaaaccggcctttttgtcgggcgaacaaaagaaagcaattgtggatctgcttttcaaaaccaaccgaaaagtaactgtgaagcagctgaaagaagattatttcaaaaaaatagaatgctttgatagtgtggaaatttcgggtgtggaagatcgttttaacgcgtcgctgggcacttaccatgatttactcaaaattattaaagataaagattttttagataacgaagaaaacgaagatatcctggaggatattgtgctgaccttaactctgtttgaagatagagagatgattgaggaacgtttgaaaacctatgcgcacctttttgatgataaggttatgaaacaattgaaacgccggcgctatacgggctggggtcgcttaagccgaaaattaattaacggcattagagataagcagagcgggaaaaccatactggattttttaaaatcggatggctttgcaaaccggaactttatgcaactaatccatgatgatagtttaacctttaaagaagacattcagaaagcccaggttagcggtcagggggatagtctgcatgaacatattgccaacctggcgggctccccagcgattaaaaaaggcattctgcaaacggtaaaagtggtggatgaattagtcaaagtaatgggaaggcataagccggaaaacatcgtgattgaaatggcccgcgaaaaccaaaccacgcagaaggggcaaaaaaactcacgagagcgcatgaaacgaatcgaagaaggcatcaaagaactgggtagtcaaattttgaaagagcatccagtggaaaacacgcagttacagaacgaaaagctttatctttattatcttcagaacggtcgtgacatgtatgttgaccaggaactggatattaaccgcctgagtgattatgatgtcgatcacattgtgccgcagagtttcttgaaagacgattcgatagacaacaaggtcctgacacgcagcgataaaaaccgcggcaaatcagataatgtgccgagtgaagaagtagtcaaaaagatgaaaaattattggcgtcagttgctcaatgcaaagctgatcacgcagcgcaagtttgataacctgacaaaagcggaacgcggtggcttaagtgaattggataaagcgggctttatcaaacggcagttagtggaaacgcggcagatcacgaagcatgttgcccagattttagatagtcggatgaacacgaaatacgatgaaaacgataaattgattcgagaggtgaaagttattactctgaaaagcaaactggtgagcgacttccgaaaagatttccagttctataaagtacgcgagattaataactaccatcatgcacatgatgcttatctcaacgcagtcgtgggtacggcgttaattaagaaatatccgaaattggaatcagagtttgtctatggcgattataaagtgtatgatgtgcgcaaaatgattgcgaagtcggagcaggaaatagggaaagccactgccaaatatttcttttacagcaacatcatgaatttcttcaaaaccgaaattaccttggccaacggtgagattcggaaacggccactcatcgaaacgaacggagaaacgggtgaaattgtctgggataaaggacgagattttgcaaccgttcggaaagtattgtccatgcctcaggtcaacattgtcaagaaaaccgaagtacaaaccgggggtttctccaaggagtcgattctgccgaaacgcaactcagacaagttgattgcgcgcaaaaaagactgggatccgaaaaaatatggcggcttcgattccccgacagtagcgtactcggtcctcgtcgtggcgaaggtcgaaaaagggaaatccaagaagctgaaatccgtgaaagagctgctcgggatcaccatcatggagcgctcctccttcgagaagaaccccatcgacttcctggaggcgaagggctacaaggaggtgaagaaggacctgatcatcaagctccccaagtactccctctttgagctggagaacggccgcaagaggatgctcgcaagtgcaggtgaattacaaaaaggtaatgaactagcgctaccgtccaaatatgttaactttctgtatctggcgagtcattatgaaaagttaaagggcagtccggaagataatgaacagaaacagttatttgttgagcaacataagcattatctggatgagattattgagcagatcagtgaatttagcaagcgcgtgattctggccgatgcaaacctggataaagtgttgagtgcctataataaacatcgtgacaaaccgatacgcgaacaggccgaaaacattattcatctgtttacattaacaaacttgggtgcgcctgcggcgtttaaatattttgataccaccattgatcgcaaacgatatacaagcaccaaagaagtgctggatgcaacgttgatccatcagtctatcacgggcttgtatgaaacccggattgatttaagtcaactcggtggcgactaagcccggccgctactagtaacaaaaaacccctagccccccgttttatatcactgcccgctttccagtcgggaaacctgtcgtgccagctgcattaatgaatcggccaacgcgcggggagaggcggtttgcgtattgggcgccagggtggtttttcttttcaccagtgagactggcaacagctgattgcccttcaccgcctggccctgagagagttgcagcaagcggtccacgctggtttgccccagcaggcgaaaatcctgtttgatggtggttaacggcgggatataacatgagctatcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcctcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcatattcaccaccctgaattgactgtcttccgggcgctatcatgccataccgcgaaaggttttgcgccattcgatggcgcgccgcgccctgcgctagcatgcctatttgtttatttttctaaatacattcaaatatgtatccgctcatgagacaataaccctgataaatgcttcaataatattgaaaaaggaacagtatgagccatattcaacgggaaacgtcttgctccaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctctcccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagccttagttagttagattagcagaaagtcaaaagcctccgaccggaggcttttgactaaaacttcccttggggttatcattggggctcactcaaaggcggtaatcagataaaaaaaatccttagctttcgctaaggatgatttctgccagatgtgtataagagacagctgggcgcgccccatgtcagccgttaagtgttcctgtgtcactcaaaattgctttgagaggctctaagggcttctcagtgcgttacatccctggcttgttgtccacaaccgttaaaccttaaaggctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatcggcgcgccggccggcctacggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaaggacaaggccctgcgctagcatgcctatttgtttatttttctaaatacattcaaatatgtatccgctcatgagacaataaccctgataaatgcttcaataatattgaaaaaggaacagtatgagtattcaacatttccgtgtcgcccttattcccttttttgcggcattttgccttcctgtttttgctcacccagaaacgctggtgaaagtaaaagatgccgaagatcagttgggtgcacgtgtgggttacatcgaactggacctcaacagcggtaagattcttgagagttttcgccccgaagaacgtttcccaatgatgagcacttttaaagttctgctctgtggcgcggtattatcccgtattgacgccgggcaagagcaactcggtcgccgcatacactattctcagaatgacttggttgagtactcaccagtcacagaaaagcatcttacggacggcatgacagtacgcgaattatgcagcgctgccataaccatgagtgataacacggcggccaacttacttctgacaacgatcggaggaccgaaggagcttaccgcttttttgcacaacatgggtgatcatgtaactcgccttgatcgttgggaaccggagctgaatgaagccataccaaacgacgagcgtgacaccacgatgcctgtagctatggcaacaacgttgcgcaaactcttaactggcgaacttcttactctcgcttcccggcaacaattaatagactggatggaggcggataaagttgcaggaccacttctgcgctcggcccttccggctggctggtttattgctgataaatctggagccggtgagcgtgggtcccgcggtattattgcagccctggggccagatggtaagccctcccgtatcgtagttatctacacgacggggagccaggcaactatggacgaacgtaatcgccagatcgctgagataggtgcctccctgattaagcattggtaagccttagattttaatgccctgcgccatcaggtctttcgcggccagaaagccatccagtttgctttgcagcgcttcccaaccttcccacagcgcaccccagctcgcaatgccggtacgtttgctatccataaagccgcccagacgcgcaatcgccatatacgcccattgcaggctgcccgctttttctttgcgtttgcgtttgcctttatccagatagcccagcagttggcattcatccggggtcagcacggtttccgcgctctggctttcaacgtgttccgcttctttcagcaggccctgcgcacgcagtgcttgcggcggagtaaaagattcacgcagttgcagcagacgcaccgccacaaagctcagaatgctcaccatacgttccaggttatccggttcttccatacgctgacgttccgcacccgcacccgttttccacgctttgtgaaattcttcaatgcgccaacgatgggtataaatatcaatcacacgcagcgcttgggccagactttccaccggctcgctggtcagcagcagccatttcagcggggtttcgcctttcggcggattaatttcttcggccagcaccgcgttcagggtaatgttgccctgtttcagggtaatacggccgctacgcaggctcaggctcgctttacgcgccggacggtttttacgtttgccacgtttatccaccacgcctttctgcggaatgctaatctgatagccgcccagttccggctggtttttcaggtgatcatacagatacaggccgctttccacatctttacgcggatgtttgctacgcaccacaaaacgttcgttatgggccagtttatcttgcagatacgcatgaatatccgcttcacgatcgcacaccgcaatcacgttgctcatcatgctgcccattctcagacgcgaagttgcagcagcggccagccatttgccgctttctttttcatccgcatccgccggatcatccggacgcatccaccattcttgatgcagcaggcccacggtacgaaaggtggtcgcttccagcagcagcacgctatgcacccaccaaccacggcttttatcctgaatgctacccagtttgcccagttcttccgccacctgatgacgatagctcagagaggtggtatcttcaattgccagcagttccggaaattcctgggccagtttcacggtctgcatggcacccgctttacgaatcgcttccgcgctcacgttcggattacgaataaaacgatacgcgccttcctgcatggctttgctgccttcgctgctaatggtaatgcttttgccgctatatttggccagttgcgccgcaacattcaccagacgcgcggtacgacgcggatcacccagcgcagcactagaaaacacgcttttcgcccaatccgccgcacgatgcagtgcactggtaatcatgaacgttaccatgttaggaggtcacatggaacatcagattctggaaaacgggaaaggttccgttcaggacgctacttgtgtagtttaaaccagctgtctcttatacacatctgcctccggcaaaaaaacgggcaaggtgtcaccaccctgccctttttctttaaaaccgaaaagattacttcgcgtttgccacctgacgtctaagaaaaggaatattcagcaatttgcccgtgccgaagaaaggcccacccgtgaaggtgagccagtgagttgattgctacgtaattagttagttaggcc

SEQ ID NO: 20 - DNA, 11077 bp, unknown
  Synthetic
aattgtgagcggataacaattacgagcttcatgcacagtgaaatcatgaaaaatttatttgctttgtgagcggataacaattataatatgtggaattgtgagcgctcacaattccacaacggtttccctctagaaataattttgtttaacttttgccctataaccagaccgttcagcgttttagagctagaaatagcaagttaaaataaggctagtccgttatcaacttgaaaaagtggcaccgagtcggtggtgcgccccaggcatcaaataaaacgaaaggctcagtcgaaagactgggcctttcgttttatctgttgtttgtcggtgaacgctctctactagagtcacactggctcaccttcgggtgggcctttctgcgtttatagcccacagcaaacaccacgtcgaccctatcagctgcgtgctttctatgagtcgttgctgcataacttgacaattaatcatccggctcgtaatgtttgtggagccgggcccaagttcacttaaaaaggagatcaacaatgaaagcaattttcgtactgaaacatcttaatcatgctaaggaggttttctaatggtaagcaagggcgaggagaataacatggccatcatcaaggagttcatgcgcttcaaggtgcacatggagggcagtgtgaacggccacgagttcgagatcgagggcgagggcgagggccgtccctacgaggcctttcagaccgctaagctgaaggtgaccaagggtggccccctgcccttcgcctgggacatcctgtcccctcagttcatgtacggctccaaggtctacattaagcacccagccgacatccccgactacttcaagctgtccttccccgagggcttcaggtgggagcgcgtgatgaacttcgaggacggcggcattattcacgttaaccaggacagttccctgcaagacggcgtgttcatctacaaggtgaagctgcgcggcaccaacttccccagtgacggccccgtaatgcagaagaagaccatgggctgggaggccagtgaggagcggatgtaccccgaggacggcgccctgaagtctgagatcaaaaagaggctgaagctgaaggacggcggccactacgccgccgaggtcaagaccacctacaaggccaagaagcccgtgcagctgcccggcgcctacatcgtcgacatcaagttggacatcgtgtcccacaacgaggactacaccatcgtggaacagtacgaacgcgccgagggccgccactccaccggcggcatggacgagctgtacaaggccgacaagaaatactcgatagggctggatatcgggaccaactccgtcggttgggctgttatcacggatgaatataaggtgcccagcaaaaagttcaaggtgctaggtaacaccgaccggcacagtatcaaaaaaaacttgataggagcgttgctgtttgacagtggtgagaccgctgaagctacgcgccttaaaaggaccgcgcgccgtagatatacccgcaggaagaaccgcatttgctatctccaagagattttttcgaacgagatggctaaagtagatgatagtttctttcatcgattggaagaaagctttttagttgaagaagacaagaagcatgaacgccatccaatttttggtaacatagtagatgaagtggcgtatcatgagaaatatccgacgatctatcatctacgaaaaaaattagtagatagcacggataaagctgatttacggctgatctatttagcactggctcatatgattaagtttcgcggccattttttaattgagggtgatctgaacccagataacagtgatgttgacaaactctttatccaattagtacagacttacaaccagctgtttgaagaaaatccaattaatgccagtggtgtagatgcgaaagctattttgagcgcccgattaagtaaatcgcgtcgactggaaaaccttattgcgcaacttcctggcgagaagaaaaacgggctgtttggaaaccttattgcgttatcgttaggcttaactccaaactttaaatcgaactttgatttagccgaagatgcgaaactgcaattgtcgaaagatacgtacgatgatgatctggataacctgttagctcagattggtgatcagtatgcggatttatttttagccgcgaagaacctgtcggatgcgattctgttgtcggatatcctccgtgtaaacacggaaataacgaaggcgcctctctcggcgtcgatgattaaacggtacgatgaacatcatcaggacttaacgttgctgaaagcgctggtgcgacagcagttgccggaaaagtataaagaaatcttttttgatcagtcgaaaaatggttatgccggctatattgatggaggtgcgtcccaggaagaattttataaatttatcaaaccgattctggaaaaaatggatggcacggaggaactgttggttaaactcaaccgcgaagatttgctacggaagcagaggacttttgacaatgggagcattcctcatcagattcacttgggcgagctacatgcgattttgcgtcgtcaggaagacttttatccgtttctgaaagacaaccgcgagaagattgaaaaaatcttgacgtttcgaatcccatattatgtgggcccgttagctcgcgggaacagtcgctttgcctggatgacgaggaagtcggaagaaaccattactccgtggaactttgaagaagtggtcgataaaggcgcgtcggcgcagtcgtttattgaacggatgaccaattttgataaaaacttgccgaacgaaaaagtactcccgaaacatagtttattgtatgagtattttacagtgtataatgaattaaccaaggtcaaatatgtgacggaaggtatgcgaaaaccggcctttttgtcgggcgaacaaaagaaagcaattgtggatctgcttttcaaaaccaaccgaaaagtaactgtgaagcagctgaaagaagattatttcaaaaaaatagaatgctttgatagtgtggaaatttcgggtgtggaagatcgttttaacgcgtcgctgggcacttaccatgatttactcaaaattattaaagataaagattttttagataacgaagaaaacgaagatatcctggaggatattgtgctgaccttaactctgtttgaagatagagagatgattgaggaacgtttgaaaacctatgcgcacctttttgatgataaggttatgaaacaattgaaacgccggcgctatacgggctggggtcgcttaagccgaaaattaattaacggcattagagataagcagagcgggaaaaccatactggattttttaaaatcggatggctttgcaaaccggaactttatgcaactaatccatgatgatagtttaacctttaaagaagacattcagaaagcccaggttagcggtcagggggatagtctgcatgaacatattgccaacctggcgggctccccagcgattaaaaaaggcattctgcaaacggtaaaagtggtggatgaattagtcaaagtaatgggaaggcataagccggaaaacatcgtgattgaaatggcccgcgaaaaccaaaccacgcagaaggggcaaaaaaactcacgagagcgcatgaaacgaatcgaagaaggcatcaaagaactgggtagtcaaattttgaaagagcatccagtggaaaacacgcagttacagaacgaaaagctttatctttattatcttcagaacggtcgtgacatgtatgttgaccaggaactggatattaaccgcctgagtgattatgatgtcgatcacattgtgccgcagagtttcttgaaagacgattcgatagacaacaaggtcctgacacgcagcgataaaaaccgcggcaaatcagataatgtgccgagtgaagaagtagtcaaaaagatgaaaaattattggcgtcagttgctcaatgcaaagctgatcacgcagcgcaagtttgataacctgacaaaagcggaacgcggtggcttaagtgaattggataaagcgggctttatcaaacggcagttagtggaaacgcggcagatcacgaagcatgttgcccagattttagatagtcggatgaacacgaaatacgatgaaaacgataaattgattcgagaggtgaaagttattactctgaaaagcaaactggtgagcgacttccgaaaagatttccagttctataaagtacgcgagattaataactaccatcatgcacatgatgcttatctcaacgcagtcgtgggtacggcgttaattaagaaatatccgaaattggaatcagagtttgtctatggcgattataaagtgtatgatgtgcgcaaaatgattgcgaagtcggagcaggaaatagggaaagccactgccaaatatttcttttacagcaacatcatgaatttcttcaaaaccgaaattaccttggccaacggtgagattcggaaacggccactcatcgaaacgaacggagaaacgggtgaaattgtctgggataaaggacgagattttgcaaccgttcggaaagtattgtccatgcctcaggtcaacattgtcaagaaaaccgaagtacaaaccgggggtttctccaaggagtcgattctgccgaaacgcaactcagacaagttgattgcgcgcaaaaaagactgggatccgaaaaaatatggcggcttcgattccccgacagtagcgtactcggtcctcgtcgtggcgaaggtcgaaaaagggaaatccaagaagctgaaatccgtgaaagagctgctcgggatcaccatcatggagcgctcctccttcgagaagaaccccatcgacttcctggaggcgaagggctacaaggaggtgaagaaggacctgatcatcaagctccccaagtactccctctttgagctggagaacggccgcaagaggatgctcgcaagtgcaggtgaattacaaaaaggtaatgaactagcgctaccgtccaaatatgttaactttctgtatctggcgagtcattatgaaaagttaaagggcagtccggaagataatgaacagaaacagttatttgttgagcaacataagcattatctggatgagattattgagcagatcagtgaatttagcaagcgcgtgattctggccgatgcaaacctggataaagtgttgagtgcctataataaacatcgtgacaaaccgatacgcgaacaggccgaaaacattattcatctgtttacattaacaaacttgggtgcgcctgcggcgtttaaatattttgataccaccattgatcgcaaacgatatacaagcaccaaagaagtgctggatgcaacgttgatccatcagtctatcacgggcttgtatgaaacccggattgatttaagtcaactcggtggcgactaagcccggccgctactagtaacaaaaaacccctagccccccgttttatatcactgcccgctttccagtcgggaaacctgtcgtgccagctgcattaatgaatcggccaacgcgcggggagaggcggtttgcgtattgggcgccagggtggtttttcttttcaccagtgagactggcaacagctgattgcccttcaccgcctggccctgagagagttgcagcaagcggtccacgctggtttgccccagcaggcgaaaatcctgtttgatggtggttaacggcgggatataacatgagctatcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcctcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcatattcaccaccctgaattgactgtcttccgggcgctatcatgccataccgcgaaaggttttgcgccattcgatggcgcgccgcgccctgcgctagcatgcctatttgtttatttttctaaatacattcaaatatgtatccgctcatgagacaataaccctgataaatgcttcaataatattgaaaaaggaacagtatgagccatattcaacgggaaacgtcttgctccaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctctcccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagccttagttagttagattagcagaaagtcaaaagcctccgaccggaggcttttgactaaaacttcccttggggttatcattggggctcactcaaaggcggtaatcagataaaaaaaatccttagctttcgctaaggatgatttctgccagatgtgtataagagacagctgggcgcgccccatgtcagccgttaagtgttcctgtgtcactcaaaattgctttgagaggctctaagggcttctcagtgcgttacatccctggcttgttgtccacaaccgttaaaccttaaaggctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatcggcgcgccggccggcctacggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaaggacaaggccctgcgctagcatgcctatttgtttatttttctaaatacattcaaatatgtatccgctcatgagacaataaccctgataaatgcttcaataatattgaaaaaggaacagtatgagtattcaacatttccgtgtcgcccttattcccttttttgcggcattttgccttcctgtttttgctcacccagaaacgctggtgaaagtaaaagatgccgaagatcagttgggtgcacgtgtgggttacatcgaactggacctcaacagcggtaagattcttgagagttttcgccccgaagaacgtttcccaatgatgagcacttttaaagttctgctctgtggcgcggtattatcccgtattgacgccgggcaagagcaactcggtcgccgcatacactattctcagaatgacttggttgagtactcaccagtcacagaaaagcatcttacggacggcatgacagtacgcgaattatgcagcgctgccataaccatgagtgataacacggcggccaacttacttctgacaacgatcggaggaccgaaggagcttaccgcttttttgcacaacatgggtgatcatgtaactcgccttgatcgttgggaaccggagctgaatgaagccataccaaacgacgagcgtgacaccacgatgcctgtagctatggcaacaacgttgcgcaaactcttaactggcgaacttcttactctcgcttcccggcaacaattaatagactggatggaggcggataaagttgcaggaccacttctgcgctcggcccttccggctggctggtttattgctgataaatctggagccggtgagcgtgggtcccgcggtattattgcagccctggggccagatggtaagccctcccgtatcgtagttatctacacgacggggagccaggcaactatggacgaacgtaatcgccagatcgctgagataggtgcctccctgattaagcattggtaagccttagattttaatgccctgcgccatcaggtctttcgcggccagaaagccatccagtttgctttgcagcgcttcccaaccttcccacagcgcaccccagctcgcaatgccggtacgtttgctatccataaagccgcccagacgcgcaatcgccatatacgcccattgcaggctgcccgctttttctttgcgtttgcgtttgcctttatccagatagcccagcagttggcattcatccggggtcagcacggtttccgcgctctggctttcaacgtgttccgcttctttcagcaggccctgcgcacgcagtgcttgcggcggagtaaaagattcacgcagttgcagcagacgcaccgccacaaagctcagaatgctcaccatacgttccaggttatccggttcttccatacgctgacgttccgcacccgcacccgttttccacgctttgtgaaattcttcaatgcgccaacgatgggtataaatatcaatcacacgcagcgcttgggccagactttccaccggctcgctggtcagcagcagccatttcagcggggtttcgcctttcggcggattaatttcttcggccagcaccgcgttcagggtaatgttgccctgtttcagggtaatacggccgctacgcaggctcaggctcgctttacgcgccggacggtttttacgtttgccacgtttatccaccacgcctttctgcggaatgctaatctgatagccgcccagttccggctggtttttcaggtgatcatacagatacaggccgctttccacatctttacgcggatgtttgctacgcaccacaaaacgttcgttatgggccagtttatcttgcagatacgcatgaatatccgcttcacgatcgcacaccgcaatcacgttgctcatcatgctgcccattctcagacgcgaagttgcagcagcggccagccatttgccgctttctttttcatccgcatccgccggatcatccggacgcatccaccattcttgatgcagcaggcccacggtacgaaaggtggtcgcttccagcagcagcacgctatgcacccaccaaccacggcttttatcctgaatgctacccagtttgcccagttcttccgccacctgatgacgatagctcagagaggtggtatcttcaattgccagcagttccggaaattcctgggccagtttcacggtctgcatggcacccgctttacgaatcgcttccgcgctcacgttcggattacgaataaaacgatacgcgccttcctgcatggctttgctgccttcgctgctaatggtaatgcttttgccgctatatttggccagttgcgccgcaacattcaccagacgcgcggtacgacgcggatcacccagcgcagcactagaaaacacgcttttcgcccaatccgccgcacgatgcagtgcactggtaatcatgaacgttaccatgttaggaggtcacatggaacatcagattctggaaaacgggaaaggttccgttcaggacgctacttgtgtagtttaaaccagctgtctcttatacacatctgcctccggcaaaaaaacgggcaaggtgtcaccaccctgccctttttctttaaaaccgaaaagattacttcgcgtttgccacctgacgtctaagaaaaggaatattcagcaatttgcccgtgccgaagaaaggcccacccgtgaaggtgagccagtgagttgattgctacgtaattagttagttaggcc